(19) United States
(12) Patent Application Publication
(10) Pub. No.: US 2026/0263372 A1
(43) Pub. Date: Sep. 10, 2026

(54) COMPOSITIONS AND METHODS

(71) Applicant: BioNTech SE, Mainz (DE)

(72) Inventors: Kaushik Thanki, Wiesbaden (DE); Larry Kwesi Sarpong, Mainz (DE); Heeralal Bassi, Mainz (DE); Hossam Hefesha, Wiesbaden (DE); Heinrich Haas, Mainz (DE)

(21) Appl. No.: 19/151,071

(22) PCT Filed: Jan. 30, 2024
(86) PCT No.: PCT/EP2024/052251

§ 371 Date: Jul. 25, 2025

(30) Foreign Application Priority Data
Jan. 31, 2023 (EP) 23154251.5
Dec. 12, 2023 (EP) 23216002.8

(51) Int. Cl. A61K 9/51; A61K 9/19; A61K 39/00

(52) U.S. Cl. CPC A61K 9/5123; A61K 9/19; A61K 9/5192; A61K 39/0011; A61K 2039/53

(57) ABSTRACT

An aqueous dispersion having an aqueous mobile phase and a dispersed phase; wherein the dispersed phase comprises a lipid mixture including a cationically ionisable lipid; and the aqueous mobile phase comprises an anion of an aqueous acid; wherein the aqueous dispersion is substantially free of inorganic cations, organic solvents and RNA, is described. Methods of preparing the aqueous dispersion, nucleic acid-lipid particles and methods of preparing them using the aqueous dispersion, and their use in medicine are disclosed.

### TECHNICAL FIELD

The present disclosure relates generally to lipid-based formulations suitable for bearing nucleic acid, in particular RNA, to lipid particles including such nucleic acids, to aqueous lipid dispersions capable of receiving the nucleic acid, and to methods for producing them, in particular such methods which do not involve the use of organic solvents.

### BACKGROUND TO THE INVENTION

The traditional manufacturing route for the preparation of nucleic acid-containing lipid nanoparticles proceeds by a one-step process of mixing in one-part nucleic acid (such as RNA) in an aqueous buffer with three-part lipid mixture dissolved in an organic solvent. Although much success has been demonstrated with this route, the established process typically suffers from the complexity of the manufacturing process, such as requiring subsequent tangential flow filtration (TFF) steps to remove the organic solvents used in the dissolution of the lipids. This further processing increases the process's complexity and eventually increases the manufacturing lead time and the associated costs. Furthermore, this approach is typically unfavourable for individualized patient therapy since a large number of batches needs to be manufactured with a very short turn-around time. The use of organic solvents in manufacturing RNA lipid nanoparticle formulations, where longer processing times are required, could also be a demerit when working with compounds that have limited chemical stability.

WO 2018/089801 describes a method for preparing empty lipid nanoparticles, in which lipids dissolved in ethanol are mixed with a citrate buffer at pH 4.5, followed by purification using tangential flow filtration wherein buffer exchange occurs. These pre-formed empty lipid nanoparticles can then be mixed with mRNA to create loaded lipid nanoparticles in which the mRNA is encapsulated. WO 2020/047061 similarly describes a method for preparing empty lipid nanoparticles, in which lipids dissolved in ethanol are mixed with a citrate buffer at pH 4.5, followed by buffer exchange (e.g., by tangential flow filtration) to produce empty lipid nanoparticles in 10% wt/vol trehalose buffer. These pre-formed empty lipid nanoparticles can then be mixed with mRNA to create loaded lipid nanoparticles in which the mRNA is encapsulated.

WO2022/032087 describes methods of preparing an empty-lipid nanoparticle solution (empty-LNP solution), comprising: i) a nanoprecipitation step, comprising: i-a) mixing a lipid solution comprising an ionizable lipid, a structural lipid, and a phospholipid, with an aqueous buffer solution comprising a first buffering agent, thereby forming an intermediate empty-lipid nanoparticle solution (intermediate empty-LNP solution) comprising an intermediate empty nanoparticle (intermediate empty LNP); i-b) holding the intermediate empty-LNP solution for a residence time; and i-c) adding a diluting solution to the intermediate empty-LNP solution, thereby forming the empty-LNP solution. The empty-LNP solution may be further processed to produce an empty-LNP formulation. Methods of producing loaded LNPs by mixing the empty-LNP solution or empty-LNP formulation with a nucleic acid are also described.

However, although ethanol, citrate buffer, and other destabilizing agents may be absent during the addition of mRNA, the formation of the empty lipid nanoparticles in all of these documents uses a buffer, particularly a citrate buffer or an acetate buffer. The inorganic ions present in such buffers (e.g., citrate buffers) are thought to destabilize the colloidal properties of the lipid nanoparticle formulation, and are therefore detrimental to formulation stability.

WO2022/069632 describes a method for preparing RNA lipoplex particles for delivery of RNA to target tissues. The methods described in this document employs solely cationic lipids, as defined herein. This document does not therefore disclose a method for producing aqueous dispersions, such as pre-LNPs, or nucleic acid-lipid particles, wherein the lipid is a cationically ionizable lipid as defined herein.

WO2011/144745 describes a method for preparing liposomes capable of being loaded with pharmaceutically and/or diagnostically active agents and/or cosmetic agents which are substantially solubilized by the liposomal membranes. The methods described in this document employ solely cationic lipids, as defined herein. This document does not therefore disclose a method for producing aqueous dispersions, such as pre-LNPs, or nucleic acid-lipid particles, wherein the lipid is a cationically ionizable lipid as defined herein.

WO 2022/101471 and WO 2022/101486 describe pharmaceutical compositions comprising lipid nanoparticles and mRNA, and methods for preparing and storing them. Specifically, these documents describe a lipid mix of DODMA, DOPE, cholesterol and C₁₆-PEG₂₀₀₀-ceramide in a molar ratio 40:10:48:2, in ethanol. These documents describe mixing of liposomes composed of this lipid mix in 5 mM acetic acid aqueous solution with RNA (10 mM HEPES, 0.1 mM EDTA, pH 7.0) to produce RNA-lipoplexes, which can then be diluted in a sucrose-containing buffer to a final sucrose concentration of 10%. These documents do not describe the lipid mix in aqueous solution in the absence of RNA comprising a cryoprotectant such as sucrose. Furthermore, this document does not teach or suggest the step of freezing or lyophilize the resulting aqueous dispersion and the consequent advantages for manufacturing personalized therapies.

WO2021/155274 describes a method of preparing an empty lipid nanoparticle (empty-LNP) solution comprising an empty lipid nanoparticle, comprising mixing a lipid solution with solution comprising a first buffering agent, thereby forming the empty-LNP solution comprising the empty LNP, wherein the empty-LNP solution comprises an acetate buffer and has a pH in the range of about 4.6 to about 6.0. The empty LNP solution may then be mixed with RNA or other nucleic acids to produce a loaded LNP in which the lipids encapsulate the RNA. However, for similar reasons as outlined above in relation to WO 2020/047061 and WO 2018/089801, the inorganic ions present in the acetate buffers used in the methods described in this document are found to be detrimental to the stability of the lipid nanoparticle formulation.

### SUMMARY OF THE INVENTION

In a first aspect, the present disclosure provides an aqueous dispersion having an aqueous mobile phase and a dispersed phase;
wherein:
the dispersed phase comprises a lipid mixture including a cationic or cationically ionisable lipid; and
the aqueous mobile phase comprises an anion of an aqueous acid;
wherein the aqueous dispersion is substantially free of inorganic cations, organic solvents and RNA, and
wherein the aqueous mobile phase comprises a cryoprotectant.

In one embodiment of this aspect, the present disclosure provides an aqueous dispersion having an aqueous mobile phase and a dispersed phase;
wherein:
the dispersed phase comprises a lipid mixture including a cationically ionisable lipid; and
the aqueous mobile phase comprises an anion of an aqueous acid;
wherein the aqueous dispersion is substantially free of inorganic cations, organic solvents and RNA, and
wherein the aqueous mobile phase comprises a cryoprotectant.

In a second aspect, the present disclosure provides an aqueous dispersion having an aqueous mobile phase and a dispersed phase; wherein:
the dispersed phase comprises a cationic or cationically ionisable lipid; and the aqueous mobile phase comprises an anion of an aqueous acid;
wherein:
the concentration of the aqueous acid is at least 6 mM; and
the aqueous mobile phase is substantially free of inorganic cations, organic solvents and RNA.

In a third aspect, the present disclosure provides an aqueous dispersion having an aqueous mobile phase and a dispersed phase; wherein:
the dispersed phase comprises a cationic or cationically ionisable lipid; and the aqueous mobile phase comprises malate anion or succinate anion;
wherein the aqueous dispersion is substantially free of inorganic cations, organic solvents and RNA.

In a fourth aspect, the present disclosure provides a method of forming the aqueous dispersion of the first aspect, the method comprising mixing:
(i) a lipid mixture comprising a cationic or cationically ionisable lipid;
(ii) an aqueous phase comprising an aqueous acid and a cryoprotectant;
to produce the aqueous dispersion comprising an anion of the aqueous acid.

In one embodiment of the fourth aspect, the present disclosure provides a method of forming the aqueous dispersion of the first aspect, the method comprising mixing:
(i) a lipid mixture comprising a cationically ionisable lipid;
(ii) an aqueous phase comprising an aqueous acid and a cryoprotectant;
to produce the aqueous dispersion comprising an anion of the aqueous acid.

In a fifth aspect, the present disclosure provides a method of forming the aqueous dispersion of the first aspect, the method comprising:
(a) mixing:
(i) a lipid mixture comprising a cationic or cationically ionisable lipid; and
(ii) an aqueous phase comprising an aqueous acid;
to produce a first intermediate aqueous dispersion comprising an anion of the aqueous acid; and
(b) adding the cryoprotectant to the first intermediate aqueous dispersion to produce the aqueous dispersion.

In one embodiment of the fifth aspect, the present disclosure provides a method of forming the aqueous dispersion of the first aspect, the method comprising:
(a) mixing:
(i) a lipid mixture comprising a cationically ionisable lipid; and
(ii) an aqueous phase comprising an aqueous acid;
to produce a first intermediate aqueous dispersion comprising an anion of the aqueous acid; and
(b) adding the cryoprotectant to the first intermediate aqueous dispersion to produce the aqueous dispersion.

In a sixth aspect, the present disclosure provides a method of forming an aqueous dispersion comprising an anion of an aqueous acid, the method comprising:
(a) mixing:
(i) a lipid mixture comprising a cationic or cationically ionisable lipid dissolved in a water-soluble organic solvent; and
(ii) an aqueous phase;
the lipid mixture and/or the aqueous phase comprising the aqueous acid;
to produce a first intermediate acidified aqueous lipid dispersion comprising an anion of the aqueous acid;
(b) performing on the first intermediate acidified aqueous lipid dispersion a dialysis or filtration step at a pH of about 2.5 to about 5.5, to remove the organic solvent and produce a second intermediate aqueous dispersion; and
(c) adding a cryoprotectant to the second intermediate aqueous dispersion; to produce the aqueous dispersion comprising an anion of the aqueous acid.

In a seventh aspect, the present disclosure provides a method of forming an aqueous dispersion comprising an anion of an aqueous acid, the method comprising:
i) mixing a lipid mixture comprising a cationic or cationically ionisable lipid dissolved in a water-soluble organic solvent with an aqueous phase,
wherein the lipid solution and/or the aqueous phase comprises an aqueous acid, to produce a first intermediate acidified aqueous lipid dispersion comprising an anion of the aqueous acid;
ii) performing on the first intermediate acidified aqueous lipid dispersion a dialysis or filtration step at a pH of about 2.5 to about 5.5, or at a pH of 6.5 to 8.5, to remove the organic solvent and produce a second intermediate aqueous dispersion; and
iii) adding a cryoprotectant to the second intermediate aqueous dispersion; to produce the aqueous dispersion;
wherein the aqueous dispersion is substantially free of inorganic cations, organic solvents and RNA.

In an eighth aspect, the present disclosure provides a method of forming the aqueous dispersion of the second aspect, the method comprising mixing:
(i) a lipid mixture comprising a cationic or cationically ionisable lipid;
(ii) an aqueous phase comprising an aqueous acid;
wherein the concentration of the aqueous acid is at least 6 mM;
to produce the aqueous dispersion comprising an anion of the aqueous acid.

In a ninth aspect, the present disclosure provides a method of forming the aqueous dispersion of the third aspect, the method comprising mixing:
(i) a lipid mixture comprising a cationic or cationically ionisable lipid;
(ii) an aqueous phase comprising malic acid or succinic acid;
to produce the aqueous dispersion comprising malate anion or succinate anion.

In a tenth aspect, the present disclosure provides a method of forming a lipid particle containing a nucleic acid (e.g., RNA, such as mRNA), the method comprising:
i) preparing an aqueous dispersion according to the method of any one of the first to ninth aspects; and
ii) mixing the aqueous dispersion with an aqueous solution comprising a nucleic acid, to produce the lipid particle containing the nucleic acid.

In an eleventh aspect, the present disclosure provides a method of forming a lipid particle containing a nucleic acid (e.g., RNA, such as mRNA), the method comprising:
i) mixing a lipid mixture comprising a cationic or cationically ionisable lipid dissolved in a water-soluble organic solvent with an aqueous phase, wherein the lipid solution and/or the aqueous phase comprises an aqueous acid, to produce a first intermediate dispersion comprising an anion of the aqueous acid;
ii) performing on the first intermediate dispersion a dialysis or filtration step at a pH of about 2.5 to about 5.5, or at a pH of 6.5 to 8.5, to remove the organic solvent and produce a second intermediate aqueous dispersion,
iii) adding a cryoprotectant to the second intermediate aqueous dispersion, to produce an aqueous dispersion, wherein the aqueous dispersion is substantially free of inorganic cations, organic solvents and nucleic acids, and
iv) mixing the aqueous dispersion with an aqueous solution comprising a nucleic acid, to produce the lipid particle containing the nucleic acid.

In a twelfth aspect, the present disclosure provides a lipid-nucleic acid particle (e.g., a lipid-RNA particle), such as a lipid nanoparticle, obtained or obtainable by the method of the tenth or eleventh aspects.

In a thirteenth aspect, the present disclosure provides a pharmaceutical composition containing a lipid particle of the twelfth aspect and a pharmaceutical carrier.

In a fourteenth aspect, the present disclosure provides a lipid particle of the twelfth aspect for use in medicine.

In a fifteenth aspect, the present disclosure provides a lipid particle of the twelfth aspect for use in a prophylactic and/or therapeutic treatment of a disease involving an antigen and/or for use in inducing an immune response.

In a sixteenth aspect, the present disclosure provides a lipid particle of the twelfth aspect for use in treating cancer.

In a seventeenth aspect, the present disclosure provides use of a lipid particle of the twelfth aspect in the manufacture of a medicament for use in a prophylactic and/or therapeutic treatment of a disease involving an antigen and/or for use in inducing an immune response.

In an eighteenth aspect, the present disclosure provides use of a lipid particle of the twelfth aspect in the manufacture of a medicament for use in treating cancer.

In a nineteenth aspect, the present disclosure provides a method of prophylactic and/or therapeutic treatment of a disease involving an antigen and/or method of inducing an immune response in a subject in need thereof, comprising administering to the subject a lipid particle of the twelfth aspect.

In a twentieth aspect, the present disclosure provides a method of prophylactic and/or therapeutic treatment of cancer in a subject in need thereof, comprising administering to the subject a lipid particle of the twelfth aspect.

In a twenty-first aspect, the present disclosure provides a lyophilised composition comprising the aqueous dispersion of any one of the first to third aspects.

In a twenty-second aspect, the present disclosure provides a frozen composition comprising the aqueous dispersion of any one of the first to third aspects, wherein the frozen composition is at a temperature between −15° C. to −90° C.

### Advantages and Surprising Findings

It has surprisingly been found by the present inventors that the method described herein enables both the aqueous dispersion composition (lacking the nucleic acid) and the nucleic acid-lipid particle to be prepared without using organic solvents. This avoids both the risk of the organic solvents degrading chemically unstable lipids and the need for complex purification processes to remove the organic solvents. One large batch of pre-formed aqueous dispersion can be used for manufacturing several batches of nucleic acid-lipid particle (e.g., patient-specific mRNA lipid nanoparticle) formulations and could be particularly interesting in areas such as individualized immunotherapy platforms where small-scale batches of the final products are required.

Furthermore, the methods disclosed herein provide superior manufacturing advantages with compatibility in a Class D manufacturing environment. In addition, the nucleic acid-lipid particles formed with this new process have been demonstrated to have improved colloidal stability, lipid stability, and RNA integrity in both frozen and liquid conditions when compared to other classical lipid nanoparticle manufacturing routes while maintaining biological efficacy. Furthermore, the methods disclosed herein allow much flexibility to alter the properties of the formulations such as particle size, surface charge and functionalization, without affecting the process's robustness. Additionally, the nucleic acid-lipid particles manufactured by the methods disclosed can also be functionalized by ligands, in order to target specific cells, organs, etc.

In addition, in contrast to the methods described in WO 2020/047061, WO 2018/089801, WO2021/155274, and WO2022/032087, the method described herein avoids the use of the inorganic ions present in citrate and acetate buffers and therefore avoiding the detrimental effects of the inorganic ions on the nucleic acid-lipid particle formulation. Specifically, the use of malic acid or succinic acid is thought to further improve the maintenance of colloidal stability and/or RNA integrity, for example during RNA-lipid particle formation and subsequent storage. It is also surprisingly found that higher concentrations of acids, such as acetic acid at concentrations of 6 mM or higher, may provide improved colloidal properties and/or does not negatively impact particle stability and/or RNA integrity. The addition of a cryoprotectant such as sucrose to the aqueous dispersion enhances long-term stability of the lipid particles and facilitates storage in frozen conditions.

Furthermore, it has been found by the present inventors that addition of a cryoprotectant during the initial mixing step in the method of forming the aqueous dispersion confers the advantages that the step of carrying out HPLC between the tangential flow filtration (TFF) and dilution steps is not required, as there is no longer any need to measure the lipid concentration to determine the amount of cryoprotectant to add by dilution. This streamlines and shortens the manufacturing process.

In addition, and in contrast to the methods described in WO 2022/101486 and WO 2022/101471, it has been found by the present inventors that addition of a cryoprotectant during the initial mixing step in the method of forming the aqueous dispersion confers the advantages of enhanced colloidal stability. Without wishing to be bound by theory, it is considered that inclusion of cryoprotectant during the initial mixing step avoids a change in osmotic pressure at the filtration (e.g., TFF)/dialysis or dilution steps, thereby enhancing colloidal stability. Surprisingly, it has also been found that loading the nucleic acid, such as RNA, into the aqueous dispersion containing the cryoprotectant results in a workable nucleic acid-lipid particle composition: this had not been thought possible as loading the nucleic acid into a more viscous aqueous dispersion was expected to be challenging. Particularly it was surprisingly found to be possible to load nucleic acid (e.g., RNA) into an aqueous dispersion comprising a higher sucrose concentration (e.g., 20% sucrose), which allowed for preparation of nucleic acid-lipid particles in a one-step procedure, avoiding a further dilution step to add cryoprotectant. This allowed for streamlining and shortening this second step of the manufacturing process, which is beneficial for RNA integrity and particle stability. Furthermore, it had not been foreseen at the publication date of WO 2022/101486 and WO 2022/101471 that the aqueous dispersion could be frozen or lyophilized and the advantages of using a two-step method for manufacturing personalized therapies had not been suggested.

### BRIEF DESCRIPTION OF THE FIGURES

FIG. 1 (a) shows a generalized manufacturing scheme for the aqueous dispersion of the invention (also described herein as “pre-formed lipid nanoparticles” (“pre-LNPs”)); (b) shows a manufacturing scheme for an exemplary aqueous dispersion of the invention (also described herein as “pre-formed lipid nanoparticles”, i.e. before the introduction of the nucleic acid) (where IPA means isopropyl alcohol);

FIG. 2 shows an exemplary manufacturing scheme for RNA-lipid particles according to the invention;

FIG. 3 shows the stability of the aqueous dispersion according to the invention (Example 1) with grafted lipid when stored in both liquid and frozen conditions, in terms of particle size (a) and polydispersity index (b);

FIG. 4 shows the long-term stability of RNA-lipid particles according to the invention (Example 1), manufacturing according to the two-step process described herein, as a function of particle size (a), polydispersity index (b) and RNA integrity (c) at −80° C. and −20° C.;

FIG. 5 shows INF-γ ELISpot showing the higher T-cell response of drug product manufactured with the process according to the invention (designated as LNP 2) (Example 2), all formulations tested having similar lipid and N/P ratios and being administered at the same doses;

FIG. 6 shows the stability of the aqueous dispersion of the invention (Example 2) comprising DODMA with C14-Psar(23)-Ac when stored in liquid (4° C. and 25° C.) conditions, in terms of particle sizes (a) and polydispersity index (b);

FIG. 7 shows the stability of the aqueous dispersion according to the invention (Example 3) with DODMA and DMG-PEG2k at different pH with 5 mM acetic acid (a) 40 mM acetate buffer (b) and 10 mM HEPES buffer (c);

FIG. 8 shows the colloidal stability of grafted free RNA-lipid particles according to the invention (Example 4) (the formulations were prepared with an N/P ratio of 6, pH 5.5, RNA content of 0.1 mg/mL and stored in HEPES buffer with 10% (w/v) sucrose; the lipid mixture was composed of an ionizable lipid HY-501, cholesterol, and DSPC at a molar ratio of 47.5:42.5:10) monitored over a period of 3 months in both frozen and liquid conditions, both the particle size (a) and polydispersity (b) were within specifications for the period tested;

FIG. 9 shows the stability of the aqueous dispersion according to the invention (Example 6) containing Alfa-tag lipid when stored in liquid (4° C. and 25° C.) conditions, in terms of particle size (a) and polydispersity index (b);

FIG. 10 shows the particle size (a) and polydispersity index (b) analysis of functionalized RNA-lipid particles according to the invention (Example 6) subjected to two freeze thaw (FT) cycles from −20° C. to room temperature and from −80° C. to room temperature;

FIG. 11 shows the particle size and polydispersity index (PDI) of raw RNA-lipid particles according to the invention (Example 7), alfa-tagged RNA-lipid particles with a post-insertion approach and functionalized RNA-lipid particles;

FIG. 12 shows the freeze-thaw stability of the pre-LNPs manufactured and purified with acetic acid at varying concentrations (1.25, 2.5 and 5 mM) according to the invention (Example 10A);

FIG. 13 shows the freeze-thaw stability of the pre-LNPs manufactured and purified with 5 mM acetic and diluted and stored in (A) 8% w/v sucrose and (B) 12% w/v sucrose according to the invention (Example 10B);

FIG. 14 shows the freeze-thaw stability of the pre-LNPs manufactured and purified with 5 mM acetic acid diluted and stored in 10% trehalose according to the invention (Example 10C);

FIG. 15 shows the freeze-thaw stability of the pre-LNPs manufactured and purified with 5 mM acetic acid diluted and stored in 5% w/v glucose according to the invention (Example 10D);

FIG. 16 shows the freeze-thaw stability of the pre-LNPs manufactured with 2.5 mM acetic acid (FIG. 16A) or 5 mM acetic acid (FIG. 16B) according to the invention (Example 11);

FIG. 17 shows the freeze-thaw stability of the pre-LNPs manufactured and purified with malic acid at varying concentrations (2.5, 5 and 10 mM) according to the invention (Example 12);

FIG. 18 shows the freeze-thaw stability of the pre-LNPs manufactured and purified with mixture of 5 mM acetic acid plus malic acid at varying concentrations, as indicated, according to the invention (Example 12);

FIG. 19 shows the freeze-thaw stability of the pre-LNPs manufactured and purified with mixture of a) 5 mM acetic acid, 5% w/v sucrose, or b) 5 mM acetic acid, 10% w/v sucrose, according to the invention (Example 14);

FIG. 20 (comparative) shows the freeze-thaw stability of pre-LNPs manufactured and purified according to Example 15 (comparative) with (A) 5 mM acetate buffer at pH 5.0 or 5.5 mM, (B) citrate buffer with varying concentration (2.5, 5, 10 and 20 mM); (C) 30 mM succinate buffer about pH 4, (D) 30 mM malate buffer about pH 4;

FIG. 21 shows the colloidal stability of RNA-LNPs according to the invention (Example 17) over five freeze-thaw cycles;

FIG. 22 shows the long term stability for RNA-LNPs of according to the invention (Example 17);

FIG. 23 shows the particle size and PDI of freeze-thawed RNA-LNPs according to the invention (Example 18);

FIG. 24 shows the long term stability for RNA-LNPs according to the invention (Example 18);

FIG. 25 shows the particle size and PDI of freeze-thawed RNA-LNPs according to the invention (Example 19);

FIG. 26 shows the particle size and PDI liquid state RNA-LNPs according to the invention (Example 20)—for 2.5 mM acetic acid, the different storage matrices are indicated by: (a) 60 mM HEPES, 3 mM Tris, 30% sucrose, pH 6.3; (b) 50 mM Tris, 30% sucrose at pH 8.5;

FIG. 27 shows the particle size and PDI of freeze-thawed RNA-LNPs according to the invention (Example 21);

FIG. 28 shows the particle size and PDI of freeze-thawed RNA-LNPs according to the invention (Example 22);

FIG. 29 shows the long term stability for up-concentrated LNPs according to the invention (Example 23) (RNA concentrations (mg/mL): Gr A=0.1; Gr B=0.2; Gr C=0.3; Gr D=0.6; Gr D=1.3);

FIG. 30 shows the particle size and PDI of RNA-LNPs according to the invention (Example 25), manufactured from lyophilized and reconstituted pre-LNPs;

FIG. 31 shows the particle size and PDI of freeze-thawed LNPs according to the invention (Example 26);

FIG. 32 shows an exemplary simplified manufacturing scheme for RNA-LNPs according to the invention, as described in Example 27; and

FIG. 33 shows the particle size and PDI of RNA-LNPs according to the invention (Example 27).

### DETAILED DESCRIPTION

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Köbl, Eds., Helvetica Chimica Acta, CH-4010 Basel, Switzerland, (1995). The practice of the present disclosure will employ, unless otherwise indicated, conventional methods of chemistry, biochemistry, cell biology, immunology, and recombinant DNA techniques which are 25 explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Rompp Chemie Lexikon, Falbe/Regitz (Hrsg.), Georg Thieme Verlag Stuttgart, New York, 1989; Molecular Cloning: A 30 Laboratory Manual, 2nd Edition, J. Sambrook et al. eds., Cold Spring Harbor Laboratory Press, Cold Spring Harbor 1989.

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a”, “an” and “the” and similar references used in the context of describing the present disclosure (especially in the context of the claims) are to be construed to cover both the singular and the plural, unless otherwise indicated herein or clearly contradicted by the context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, such as ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. For example, with respect to a pH value, the term “about” may in preferred instances indicate deviation from the indicated numerical value by up to 0.3. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

The expression “substantially free of X”, as used herein, means that the composition described herein is free of X in such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, or less than 0.001% by weight), based on the total weight of the mixture. Specific meanings of the term “substantially free” in relation to certain components of the composition are defined herein.

“Physiological pH” as used herein refers to a pH of about 7.5 or about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

“Physiological conditions” as used herein refer to the conditions (in particular pH and temperature) in a living subject, in particular a human. Preferably, physiological conditions mean a physiological pH and/or a temperature of about 37° C.

As used in the present disclosure, “mol %” is defined as the ratio of the number of moles of one component to the total number of moles of all components, multiplied by 100.

As used in the present disclosure, “mol % of the lipid mixture” is defined as the ratio of the number of moles of that particular lipid component to the total number of moles of all lipids in the lipid mixture, multiplied by 100. In this context, in some embodiments, the term “total lipid” and/or “total lipid mixture” includes lipids and lipid-like material.

The term “hydrocarbyl” as used herein relates to a monovalent organic group obtained by removing one H atom from a hydrocarbon molecule. In some embodiments, hydrocarbyl groups are non-cyclic, e.g., linear (straight) or branched. Typical examples of hydrocarbyl groups include alkyl, alkenyl, alkynyl, cycloalkyl, aryl groups, and combinations thereof (such as arylalkyl (aralkyl), etc.). Particular examples of hydrocarbyl groups are C₁₋₄₀ alkyl (such as C₆₋₄₀ alkyl, C₆₋₃₀ alkyl, C₆₋₂₀ alkyl, or C₁₀₋₂₀ alkyl), C₂₋₄₀ alkenyl (such as C₆₋₄₀ alkenyl, C₆₋₃₀ alkenyl, or C₆₋₂₀ alkenyl) having 1, 2, or 3 double bonds, aryl, and aryl(C₁₋₆ alkyl). In some embodiments, the hydrocarbyl group is optionally substituted with one or more, such as 1, 2 or 3, such as 1 or 2, such as 1 substituents selected from List A.

The term “heterohydrocarbyl” means a hydrocarbyl group as defined above in which from 1, 2, 3, or 4 carbon atoms in the hydrocarbyl group are replaced by heteroatoms of oxygen, nitrogen, silicon, selenium, phosphorus, or sulfur, preferably O, S, or N. In one embodiment, the heterohydrocarbyl is substituted with one or more, such as 1, 2 or 3, such as 1 or 2, such as 1 substituents selected from List A.

The term “alkyl” refers to a monoradical of a saturated straight or branched hydrocarbon. Preferably, the alkyl group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkyl groups include methyl, ethyl, propyl, iso-propyl (also called 2-propyl or 1 methylethyl), butyl, iso-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, iso-heptyl, n-octyl, 2-ethyl-hexyl, n-nonyl, ndecyl, n-undecyl, n-dodecyl, n-undecyl, n-dodecyl, n-tridecyl, n-tetradecyl, n-pentadecyl, n-hexadecyl, n-heptadecyl, n-octadecyl, n-nonadecyl, n-icosyl, n-triacontyl, n-tetracontyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). In one embodiment, the alkyl is substituted with one or more, such as 1, 2 or 3, such as 1 or 2, such as 1 substituents selected from List A. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene group comprises from 1 to 40, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, carbon atoms, such as 1 to 30, such as 1 to 20 carbon atoms, such as 1 to 12 carbon atoms, such as 1 to 10 carbon atoms, such as 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1 -propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituent may be the same or different). In one embodiment, the alkylene is substituted with one or more, such as 1, 2 or 3, such as 1 or 2, such as 1 substituents selected from List A.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 40 carbon atoms, such as 2 to 30 carbon atoms, such as 2 to 20 carbon atoms, such as 2 to 12 carbon atoms, such as 2 to 10 carbon atoms, such as 2 to 8 carbon atoms, such as 2 to 6 carbon atoms or 2 to 4 carbon atoms. Thus, in a preferred embodiment, the alkenyl group comprises from 2 to 40, such as 2 to 30, such as 2 to 20, such as 2 to 12, such as 2 to 10 carbon atoms and 1, 2, 3, 4, 5, or 6 (e.g., 1, 2, 3, 4, or 5) carbon-carbon double bonds, such as comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, and the like. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). In one embodiment, the alkenyl is substituted with one or more, such as 1, 2 or 3, such as 1 or 2, such as 1 substituents selected from List A.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in a preferred embodiment, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably 5 it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. A “substituted alkenylene” means that one or more (such as 1 to the maximum number of hydrogen atoms bound to an alkenylene group, e.g., 1, 2, 3, 4, 5, 6, 7, 8, 9, or 15 up to 10, such as between 1 to 5, 1 to 4, or 1 to 3, or 1 or 2) hydrogen atoms of the alkenylene group are replaced with a substituent other than hydrogen (when more than one hydrogen atom is replaced, the substituents may be the same or different). In one embodiment, the alkenylene is substituted with one or more, such as 1, 2 or 3, such as 1 or 2, such as 1 substituents selected from List A.

The term “alkynyl” refers to a linear or branched monovalent hydrocarbon moiety having at least one carbon-carbon triple bond in which the total carbon atoms may be six to forty, such as six to thirty, typically six to twenty, such as six to eighteen. Alkynyl groups can optionally have one or more carbon-carbon triple bonds. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different). In one embodiment, the alkynyl is substituted with one or more, such as 1, 2 or 3, such as 1 or 2, such as 1 substituents selected from List A.

The terms “cycloalkyl” and “cycloalkenyl” represents cyclic non-aromatic versions of “alkyl” and “alkenyl” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkyl groups include cyclopropyl, cyclobutyl, cyclopentyl, cyclohexyl, cycloheptyl, cyclooctyl, cyclononyl, cyclodecyl, and adamantyl. Exemplary cycloalkenyl groups include cyclopropenyl, cyclobutenyl, cyclopentenyl, cyclohexenyl, cycloheptenyl, cyclooctenyl, cyclononenyl, and cyclodecenyl. The cycloalkyl or cycloalkenyl group may consist of one ring (monocyclic), two rings (bicyclic), or more than two rings (polycyclic). A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). In one embodiment, the cycloalkyl or cycloalkenyl is substituted with one or more, such as 1, 2 or 3, such as 1 or 2, such as 1 substituents selected from List A.

The terms “cycloalkylene” and “cycloalkenylene” represents cyclic non-aromatic versions of “alkylene” and “alkenylene” with preferably 3 to 40, such as 3 to 30, such as 3 to 20, such as 3 to 14 carbon atoms, such as 3 to 12 or 3 to 10 carbon atoms, i.e., 3, 4, 5, 6, 7, 8, 9, 10, 11, 12, 13, or 14 carbon atoms (such as 3, 4, 5, 6, 7, 8, 9, or 10 carbon atoms), more preferably 3 to 7 carbon atoms. Exemplary cycloalkylene groups include cyclopropylene, cyclobutylene, cyclopentylene, cyclohexylene, and cycloheptylene. Exemplary cycloalkylenene groups include cyclopentenylene and cyclohexenylene.

The term “aryl” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, 5 or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). In one embodiment, the aryl is substituted with one or more, such as 1, 2 or 3, such as 1 or 2, such as 1 substituents selected from List A. Examples of a substituted aryl include biphenyl, 2-fluorophenyl, 2-chloro-6-methylphenyl, anilinyl, 4-hydroxyphenyl, and methoxyphenyl (i.e., 2-, 3-, or 4-methoxyphenyl).

The term “heteroaryl” or “heteroaromatic ring” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-membered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). In one embodiment, the heteroaryl is substituted with one or more, such as 1, 2 or 3, such as 1 or 2, such as 1 substituents selected from List A.

The term “heterocyclyl” or “heterocyclic ring” means a cycloalkyl group as defined above in which from 1, 2, 3, or 4 carbon atoms in the cycloalkyl group are replaced by heteroatoms of oxygen, nitrogen, silicon, selenium, phosphorus, or sulfur, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the 5 maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholinyl, pyrrolidinyl, imidazolidinyl, pyrazolidinyl, piperidinyl (also called piperidyl), piperazinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). In one embodiment, the heterocyclyl is substituted with one or more, such as 1, 2 or 3, such as 1 or 2, such as 1 substituents selected from List A. The term “alkylcycloalkyl” means a cycloalkyl group, as defined above, which is substituted with an alkyl group, as defined above, the cycloalkyl portion being connected to the rest of the molecule. Each of the cycloalkyl and alkyl portions of the group may take any of the broadest or preferred meanings recited above. A “substituted alkylcycloalkyl” means that one or more (such as 1 to the maximum number of hydrogen atoms bound to a alkylcycloalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the alkylcycloalkyl is substituted with one or more, such as 1, 2 or 3, such as 1 or 2, such as 1 substituents selected from List A.

The term “cycloalkylalkyl” means an alkyl group, as defined above, which is substituted with a cycloalkyl group, as defined above, the alkyl portion being connected to the rest of the molecule. Each of the cycloalkyl and alkyl portions of the group may take any of the broadest or preferred meanings recited above. A “substituted cycloalkylalkyl” means that one or more (such as 1 to the maximum number of hydrogen atoms bound to a cycloalkylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the cycloalkylalkyl is substituted with one or more, such as 1, 2 or 3, such as 1 or 2, such as 1 substituents selected from List A.

The term “alkylcycloalkylalkyl” means an alkyl group, as defined above, which is substituted with a cycloalkyl group, as defined above, the alkyl portion being connected to the rest of the molecule and the cycloalkyl portion in turn being substituted with a further alkyl group. Each of the cycloalkyl and alkyl portions of the group may take any of the broadest or preferred meanings recited above. A “substituted alkylcycloalkylalkyl” means that one or more (such as 1 to the maximum number of hydrogen atoms bound to a alkylcycloalkylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or cycloalkyl portions of the group are replaced with a substituent other than hydrogen (when more than one hydrogen atom is replaced the substituents may be the same or different). In one embodiment, the alkylcycloalkylalkyl is substituted with one or more, such as 1, 2 or 3, such as 1 or 2, such as 1 substituents selected from List A.

The term “alkylaryl” means an aryl group, as defined above, which is substituted with an alkyl group, as defined above, the aryl portion being connected to the rest of the molecule. Each of the aryl and alkyl portions of the group may take any of the broadest or preferred meanings recited above. A “substituted alkylaryl” means that one or more (such as 1 to the maximum number of hydrogen atoms bound to an alkylaryl group, e.g., 1, 2, 3, 4, 5, 6, 7, 8, 9, or up to 10, such as between 1 to 5, 1 to 4, or 1 to 3, or 1 or 2) hydrogen atoms of either the alkyl or aryl portions of the group are replaced with a substituent other than hydrogen (when more than one hydrogen atom is replaced the substituents may be the same or different). In one embodiment, the alkylaryl is substituted with one or more, such as 1, 2 or 3, such as 1 or 2, such as 1 substituents selected from List A.

The term “arylalkyl” means an alkyl group, as defined above, which is substituted with an aryl group, as defined above, the alkyl portion being connected to the rest of the molecule. Each of the aryl and alkyl portions of the group may take any of the broadest or preferred meanings recited above. A “substituted arylalkyl” means that one or more (such as 1 to the maximum number of hydrogen atoms bound to a arylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or aryl portions of the group are replaced with a substituent other than hydrogen (when more than one hydrogen atom is replaced the substituents may be the same or different). In one embodiment, the arylalkyl is substituted with one or more, such as 1, 2 or 3, such as 1 or 2, such as 1 substituents selected from List A.

The term “alkylheteroaryl” means a heteroaryl group, as defined above, which is substituted with an alkyl group, as defined above, the heteroaryl portion being connected to the rest of the molecule. Each of the heteroaryl and alkyl portions of the group may take any of the broadest or preferred meanings recited above. A “substituted alkylheteroaryl” means that one or more (such as 1 to the maximum number of hydrogen atoms bound to an alkylheteroaryl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the alkylheteroaryl is substituted with one or more, such as 1, 2 or 3, such as 1 or 2, such as 1 substituents selected from List A.

The term “heteroarylalkyl” means an alkyl group, as defined above, which is substituted with a heteroaryl group, as defined above, the alkyl portion being connected to the rest of the molecule. Each of the aryl and alkyl portions of the group may take any of the broadest or preferred meanings recited above. A “substituted heteroarylalkyl” means that one or more (such as 1 to the maximum number of hydrogen atoms bound to a heteroarylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the heteroarylalkyl is substituted with one or more, such as 1, 2 or 3, such as 1 or 2, such as 1 substituents selected from List A.

The term “alkylheterocyclyl” means a heterocyclyl group, as defined above, which is substituted with an alkyl group, as defined above, the heteroaryl portion being connected to the rest of the molecule. Each of the heterocyclyl and alkyl portions of the group may take any of the broadest or preferred meanings recited above. A “substituted alkylheterocyclyl” means that one or more (such as 1 to the maximum number of hydrogen atoms bound to an alkylheterocyclyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heteroaryl portions of the group are replaced with a substituent other than hydrogen (when more than one hydrogen atom is replaced the substituents may be the same or different). In one embodiment, the alkylheterocyclyl is substituted with one or more, such as 1, 2 or 3, such as 1 or 2, such as 1 substituents selected from List A.

The term “heterocyclylalkyl” means an alkyl group, as defined above, which is substituted with a heterocyclyl group, as defined above, the alkyl portion being connected to the rest of the molecule. Each of the heterocyclyl and alkyl portions of the group may take any of the broadest or preferred meanings recited above. A “substituted heterocyclylalkyl” means that one or more (such as 1 to the maximum number of hydrogen atoms bound to a heterocyclylalkyl group, e.g., 1, 2, 3, 4, 5, 6, 7, 8, 9, or up to 10, such as between 1 to 5, 1 to 4, or 1 to 3, or 1 or 2) hydrogen atoms of either the alkyl or heterocyclyl portions of the group are replaced with a substituent other than hydrogen (when more than one hydrogen atom is replaced the substituents may be the same or different). In one embodiment, the heterocyclylalkyl is substituted with one or more, such as 1, 2 or 3, such as 1 or 2, such as 1 substituents selected from List A.

The term “organosulfuric acid” or “sulfate” means a compound of formula R—OSO₂—OH, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “sulfate” is used when the group is deprotonated. Depending on the pH, the sulfate group may be protonated or deprotonated (in the anionic amphiphiles as defined below, the sulfonic acid group is typically deprotonated at physiological pH).

The term “sulfonic acid” or “sulfonate” means a compound of formula R—SO₂—OH, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “sulfonate” is used when the group is deprotonated. Depending on the pH, the sulfonate group may be protonated or deprotonated (in the anionic amphiphiles as defined below, the sulfonate group is typically deprotonated at physiological pH).

The term “carboxylic acid” or “carboxylate” means a compound of formula R—CO₂H, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). The term “carboxylate” is used when the group is deprotonated. Depending on the pH, the carboxylic acid may be protonated or deprotonated (in the anionic amphiphiles as defined below, the carboxylic acid group is typically protonated at acidic pH and deprotonated at neutral or alkaline pH).

The term “dicarboxylic acid” or “dicarboxylate” means a compound of formula HO₂C—R′—CO₂H, wherein R′ is alkylene or alkenylene group (all as defined above, either in a broadest aspect or a preferred aspect). The term “dicarboxylate” is used when the group is deprotonated. Depending on the pH, the dicarboxylic acid may be protonated or deprotonated (in the anionic amphiphiles as defined below, the dicarboxylic acid group is typically protonated at acidic or neutral pH and deprotonated at alkaline pH).

The term “hydroxy carboxylic acid” or “hydroxy carboxylate” means a compound of formula R—CO₂H, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), which is substituted by one or more (preferably 1 to 5, such as 1, 2 or 3) hydroxy groups. The term “hydroxy carboxylate” is used when the group is deprotonated. Depending on the pH, the hydroxy carboxylic acid may be protonated or deprotonated (in the anionic amphiphiles as defined below, the carboxylic acid group is typically protonated at acidic pH and deprotonated at neutral or alkaline pH).

The term “ester” as used herein means a compound having the structure R—C(O)O—R′ (including its isomerically arranged structure R—OC(O)—R′, unless it is specified to the contrary). wherein R and R′ are each independently hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). When the term denotes a substituent connected to the rest of a molecule, the ester moiety may have the structure R—C(O)O— or R—OC(O)—, where R is as defined above. In one embodiment, each of both ends of the ester structure is covalently linked to a C atom of the same organic group or of two separate organic groups (e.g., an alkylene group as further component of the linker).

The term “hemiester” as used herein with respect to a functional moiety relates to an ester of a dicarboxylic acid, as defined above, where one of the carboxylic acid groups forms an ester bond with the rest of the molecule, and the other carboxylic acid group is free. Depending on the pH, the free carboxylic acid group may be protonated or deprotonated (in the anionic amphiphiles as defined below, the free carboxylic acid group is typically protonated at acidic pH and deprotonated at neutral or alkaline pH).

The term “phosphate” or “organophosphoric acid” means a compound of formula RO—P(═O)(OH)₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Depending on the pH, the phosphate group may be protonated or deprotonated (in the anionic amphiphiles as defined below, the phosphate group is typically deprotonated at physiological pH).

The term “phosphonate” or “organophosphonic acid” means a compound of formula R—P(═O)(OH)₂, wherein R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect). Depending on the pH, the phosphonate group may be protonated or deprotonated (in the anionic amphiphiles as defined below, the phosphonate group is typically deprotonated at physiological pH).

“Halo” means fluoro (—F), chloro (—Cl), bromo (—Br) or iodo (—I).

“Amine” means the group —NR₂, wherein each R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group. When both groups R are hydrogen, the amine group is a primary amine group. When one R is hydrogen and the other R is other than hydrogen, the amine group is a secondary amine group. When both groups R are other than hydrogen, the amine group is a tertiary amine group.

A “quaternary ammonium” salt is a compound containing a group —N⁺R₃, wherein each R is a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group. In contrast to some amines as defined above which are protonated only at certain pH, a quaternary ammonium salt carries a constitutive positive charge (as defined herein) at all pH.

“Hydroxyl”—means the group —OH. “Sulfhydryl”—means the group —SH. “Nitro” means the group —NO₂.

“Ether” means an oxygen atom to which two hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl groups (all as defined above, either in a broadest aspect or a preferred aspect) are attached. The ether may be a cyclic ether, wherein the two hydrocarbyl groups together form a ring, and may include dioxolane groups.

“Thioether” means a sulfur atom to which two a hydrocarbyl or heterohydrocarbyl groups, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl groups (all as defined above, either in a broadest aspect or a preferred aspect) are attached. The ether may be a cyclic thioether, wherein the two hydrocarbyl groups together form a ring, and may include dithiane groups.

“Amide” means the group —C(═O)NR(R′), wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect) and is preferably an alkyl group, such as a C₁₋₆ alkyl group.

“Hydroxylamide” means the group —C(═O)O—NR(R′), wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect).

“Sulfonamide” means the group —S(═O)₂NRR′, wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Carbamate” means the group —O—C(═O)NRR′ wherein R and R′ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Amidine” means the group —C(═NR)NR′R″ wherein R, R′ and R″ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

“Guanidine” means the group —NR—C(═NR′)NR″R′″ or ═N—C(NRR′)(NR″R′″) wherein R, R′, R″ and R′″ are each independently hydrogen or a hydrocarbyl or heterohydrocarbyl group, such as an alkyl, alkenyl, alkynyl, cycloalkyl, cycloalkenyl, cycloalkylalkyl, alkylcycloalkyl, alkylcycloalkylalkyl, aryl, alkylaryl, arylalkyl, alkylarylalkyl, alkylheteroaryl, heteroarylalkyl, alkylheterocyclyl, or heterocyclylalkyl group (all as defined above, either in a broadest aspect or a preferred aspect), and is preferably an alkyl group, such as a C₁₋₆ alkyl group.

The above definitions, when relating to any basic nitrogen atom which is protonated, may be modified by the substitution of the suffix “-ium” in accordance with normal chemical nomenclature. For example, a guanidinium group is a protonated guanidine, an ammonium group is a protonated ammonia or a protonated primary, secondary tertiary amine, an imidazolium group is a protonated imidazole, a pyridinium group is a protonated pyridine, an amidinium group is a protonated amidine, and a piperazinium group is a protonated piperazine.

“Carbohydrate” means a compound having the empirical formula Cₘ(H₂O)ₙ where m may or may not be different from n. The term “carbohydrate residue” or “carbohydrate moiety” defines a residue attached to another atom, where one hydrogen atom of the carbohydrate is replaced by a bond attached to the rest of the molecule. The carbohydrate moiety may be a monosaccharide moiety. The monosaccharide moiety may have the D- or L-configuration. Furthermore, the monosaccharide moiety may be an aldose or ketose moiety. Suitably, the monosaccharide moiety may have 3 to 8, preferably 4 to 6, more preferably 5 or 6, carbon atoms. In one embodiment, the monosaccharide moiety is a hexose moiety (i.e. it has 6 carbon atoms), examples of which include aldohexoses such as glucose, galactose, allose, altrose, mannose, gulose, idose and talose, and ketohexoses such as fructose and sorbose. Preferably, the hexose moiety is a glucose moiety.

In another embodiment, the monosaccharide moiety is a pentose moiety (i.e. it has 5 carbon atoms), such as ribose, arabinose, xylose or lyxose. Preferably, the pentose moiety is an arabinose or xylose moiety. In another embodiment, the carbohydrate may be a higher saccharide (i.e. a di-, or oligosaccharide) comprising more than one monosaccharide moiety joined together by glycoside bonds. When the monosaccharide moieties are hexose moieties, the glycoside bonds may be 1-α,1′-α glycoside bonds, 1,2′-glycoside bonds (which maybe 1-α2′ or 1′-β-2′ glycoside bonds), 1,3′-glycoside bonds (which may be 1-α-3′ or 1-β-3′-glycoside bonds), 1,4′-glycoside bonds (which may be 1-α-4′ or 1-β-4′-glycoside bonds), 1,6′-glycoside bonds (which may be 1-α-6′ or 1-β-6′-glycoside bonds), or any combination thereof. In one embodiment, the higher saccharide comprises 2 monosaccharide units (i.e. is a disaccharide). Examples of suitable disaccharides include maltose, isomaltose, isomaltulose, lactose, sucrose, cellobiose, nigerose, kojibiose, trehalose and trehalulose. In another embodiment, the higher saccharide comprises 3 to 10 monosaccharide units (i.e. is an oligosaccharide) in a chain, which may be branched or unbranched. Preferably, the oligosaccharide comprises 3 to 8, more preferably 3 to 6, monosaccharide units. Examples of suitable oligosaccharides include maltodextrin, maltotriose, maltotetraose, maltopentaose, maltohexaose, maltoheptaose, melezitose, cellotriose, cellotetraose, cellopentaose, cellohexaose and celloheptaose.

“List A” substituents are selected from the group consisting of C₁₋₆ alkyl, C₂₋₆ alkenyl, C₂₋₆ alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO₂, —OR′, —N(R′)₂, —S(O)₀₋₂R′, —S(O)₁₋₂OR′, —OS(O)₁₋₂R′, —OS(O)₁₋₂OR′, —S(O)₁₋₂N(R′)₂, —OS(O)₁₋₂N(R′)₂, —N(R′)S(O)₁₋₂R′, —N(R′)S(O)₁₋₂OR′, —C(═X¹)R′, —C(═X¹)X¹R′, —X¹C(═X¹)R′, and —X¹C(═X¹)X¹R′, wherein X¹ is independently selected from O, S, NH and N(CH₃); and each R′ is independently selected from the group consisting of H, C₁₋₄ alkyl, C₂₋₄ alkenyl, C₂₋₄ alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂(C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, A1, consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. In some embodiments, List A substituents are selected from List A2, consisting of methyl, ethyl, propyl, isopropyl, halogen (such as F, Cl, or Br), and —CF₃.

### Amino Acid

In some embodiments, the methods and compositions of the present invention, particularly the further processing steps such as the dialysis or filtration steps, the dilution or addition of storage matrix steps, and the storage steps, use an amino acid.

“Amino acid” in its broadest sense takes its normal meaning in the art of a compound containing an amine group (as defined and exemplified above, either in its broadest aspect or a preferred aspect) and a carboxylic acid group (as defined and exemplified above, either in its broadest aspect or a preferred aspect). The amino acid may contain other functional groups as defined and exemplified herein.

As is well known to the person skilled in the art, depending on the pH, amino acids can exist in a number of forms. In one embodiment, the amino acid is in zwitterionic form (i.e. wherein a proton from a carboxylic acid group is transferred to an amino group, thus leaving a negative carboxylate group and a positive ammonium group). In one embodiment, the amino acid is in neutral form (i.e. wherein both the amino group and carboxylic acid group are uncharged). In one embodiment, typically at acidic pH, the amino acid is in cationic form (i.e. wherein only the amine group is protonated, thereby having an uncharged carboxylic acid group and a positive ammonium group).

In one embodiment, typically at basic pH, the amino acid is in anionic form (i.e. wherein only the carboxylic acid group is deprotonated, thus leaving a negative carboxylate group and an uncharged amine group). Amino acids are named in this specification, as generally in the art, according to their neutral structure. The use of any particular amino acid names does not imply a limitation to the neutral structure but includes all neutral, protonated, deprotonated, and zwitterionic structures.

In one embodiment, the amino acid is an alpha amino acid (i.e. wherein the amino group is present on the carbon next to the carbon which forms the carboxylic acid group). Typically, such alpha amino acids have the general formula (in neutral structure) H₂N—CH(R)—CO₂H, wherein the group R is termed a side chain. Proline and its derivatives differ from this structure in that the nitrogen atom forms part of a pyrrolidine ring.

In one embodiment, the amino acid is a proteinogenic amino acid. Examples of proteinogenic amino acids include arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine and tryptophan.

In one embodiment, the amino acid is a substituted proteinogenic amino acid, i.e. a proteinogenic amino acid, selected from those listed above, substituted by one or more substituents selected from List A. Examples of such substituted proteinogenic amino acids include 3-hydroxyglutamic acid, 2-methyl-L-serine and O-methyl-L-serine.

In one embodiment, the amino acid is a non-proteinogenic amino acid. Examples of proteinogenic amino acids include α-aminoadipic acid, β-alanine, α-aminoisobutyric acid, β-aminoisobutyric acid, γ-aminobutyric acid, δ-aminolevulinic acid, 4-aminobenzoic acid, dehydroalanine, norvaline, alloisoleucine, allothreonine, homocysteine, homoserine, isoserine, citrulline, ornithine, homophenylalanine, 7-azatryptophan, norleucine, homoserine, sarcosine, L-beta-homoleucine, and substituted derivatives of any thereof in which the substituents are selected from List A.

In one embodiment, the amino acid is an acidic amino acid. In one embodiment, the acidic amino acid has an isoelectric point (pI), i.e. the pH at which the molecule carries no net electrical charge, of below 4. In one embodiment, the acidic amino acid is an amino acid having an acidic side chain. Examples of acidic side chains include carboxylic acid, sulfonic acid, organosulfuric acid, phosphonic acid, and phosphate, as defined and exemplified above. Preferably, the acidic amino acid is an amino acid having a carboxylic acid side chain. Examples of acidic amino acids include aspartic acid, glutamic acid, and substituted derivatives of any thereof in which the substituents are selected from List A. More preferably, the acidic amino acid is selected from the group consisting of aspartic acid, glutamic acid, 3-hydroxyglutamic acid, and alpha-aminoadipic acid.

In one embodiment, the amino acid is a neutral amino acid. In one embodiment, the neutral amino acid has an isoelectric point (pI), of between 4 and 7.8. In one embodiment, the neutral amino acid is an amino acid lacking either an acidic or a basic side chain. Examples of neutral amino acids include serine, threonine, asparagine, glutamine, cysteine, proline, alanine, valine, isoleucine, leucine, methionine, phenylalanine, tyrosine and tryptophan, and substituted derivatives of any thereof in which the substituents are selected from List A. More preferably, the neutral amino acid is selected from the group consisting of leucine and isoleucine.

In one embodiment, the amino acid is a basic amino acid. In one embodiment, the basic amino acid has an isoelectric point (pI) of above 7.8, preferably above 8.5. In one embodiment, the basic amino acid is an amino acid having a basic side chain. Examples of basic side chains include amine, amidine, and guanidine, and nitrogen-containing heteroaryl and heterocyclyl, all as defined and exemplified above. Examples of basic amino acids include arginine, histidine, lysine, and substituted derivatives of any thereof in which the substituents are selected from List A. More preferably, the basic amino acid is selected from the group consisting of arginine, histidine, and lysine.

### Nucleic Acid

The lipid particle compositions of the present application contain an active ingredient. The active ingredient is a nucleic acid. Preferably the lipid particle compositions of the present application contain RNA, such as mRNA. Typically, the lipid particle compositions described herein comprise lipid particles that encapsulate the nucleic acid. The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. In one embodiment, the nucleic acid is RNA. In one embodiment, the nucleic acid is mRNA. In one embodiment, the nucleic acid is DNA.

A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine. Nucleic acids may include one or more modified nucleosides or nucleotides. Examples of modified nucleosides or nucleotides which may be incorporated into nucleic acids include N7-alkylguanine, N6-alkyl-adenine, 5-alkyl-cytosine, 5-alkyl-uracil, and N(1)-alkyl-uracil, such as N7-C1-4 alkylguanine, N6-C1-4 alkyl-adenine, 5-C1-4 alkyl-cytosine, 5-C1-4 alkyl-uracil, and N(1)-C1-4 alkyl-uracil, preferably N7-methyl-guanine, N6-methyl-adenine, 5-methyl-cytosine, 5-methyl-uridine (m5U), pseudouridine (ψ), and N1-methyl-pseudouridine (m1Ψ).

### RNA

In some embodiments of all aspects of the disclosure, the nucleic acid is RNA. According to the present disclosure, the term “RNA” means a nucleic acid molecule which includes ribonucleotide residues. RNA typically comprises the naturally occurring nucleic acids adenosine (A), uridine (U), cytidine (C) and guanosine (G). In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides (or modified nucleosides) can be referred to as analogs of naturally occurring nucleotides (nucleosides), and the corresponding RNAs containing such altered/modified nucleotides or nucleosides (i.e., altered/modified RNAs) can be referred to as analogs of naturally occurring RNAs. A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). “RNA” includes mRNA, tRNA, ribosomal RNA (rRNA), small nuclear RNA (snRNA), self-amplifying RNA (saRNA), trans-amplifying RNA (taRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In some embodiments, “RNA” refers to mRNA. The active ingredient may be mRNA, saRNA, taRNA, or mixtures thereof. The active ingredient is preferably mRNA. In some instances, the active ingredient is not siRNA.

In a preferred embodiment, the RNA comprises an open reading frame (ORF) encoding a peptide, polypeptide or protein. Said RNA may capable of or configured to express the encoded peptide, polypeptide, or protein. For example, said RNA may be RNA encoding and capable of or configured for expressing a pharmaceutically active peptide or protein. In some embodiments, RNA is able to interact with the cellular translation machinery allowing translation of the peptide or protein. A cell may produce the encoded peptide or protein intracellularly (e.g. in the cytoplasm), may secrete the encoded peptide or protein, or may produce it on the surface. Alternatively, the RNA can be non-coding RNA such as antisense-RNA, micro RNA (miRNA) or siRNA.

mRNA

In preferred embodiments of all aspects of the disclosure, the nucleic acid is mRNA. According to the present disclosure, the term “mRNA” means “messenger-RNA” and includes a “transcript” which may be generated by using a DNA template. Generally, mRNA encodes a peptide, polypeptide or protein. As established in the art, the RNA (such as mRNA) generally contains a 5′ untranslated region (5′-UTR), a peptide/polypeptide/protein coding region and a 3′ untranslated region (3′-UTR).

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In preferred embodiments of the present disclosure, the mRNA relates to an RNA transcript which encodes a peptide, polypeptide or protein.

In some embodiments, the RNA which preferably encodes a peptide, polypeptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

In some embodiments, the RNA (such as mRNA) is produced by in vitro transcription or chemical synthesis. Preferably, the RNA (such as mRNA) is produced by in vitro transcription using a DNA template. The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)). The in vitro transcription methodology is known to the skilled person; cf., e.g., Molecular Cloning: A Laboratory Manual, 2nd Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™).

For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription. The RNA (such as mRNA) may be modified. The RNA (such as mRNA) may comprise modified nucleotides or nucleosides, such as 5-methyl-cytosine, 5-methyl-uridine (m5U), pseudouridine (ψ) or N(1)-methyl-pseudouridine (m1ψ). One or more uridine in the RNA described herein may be replaced by a modified nucleoside. The modified nucleoside may be a modified uridine. The RNA may comprise a modified nucleoside in place of at least one uridine. Preferably, the RNA may comprise a modified nucleoside in place of each uridine (e.g., all of the uridines in the RNA are replaced with a modified nucleoside). The modified nucleoside may be independently selected from pseudouridine (ψ), N1-methyl-pseudouridine (m1ψ), and 5-methyl-uridine (m5U). The modified nucleoside is preferably pseudouridine (ψ) or N1-methyl-pseudouridine (m1ψ).

In some embodiments, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”). The lipid particles containing RNA as described herein may contain mRNA, saRNA, taRNA, or mixtures thereof. The lipid particles containing RNA as described herein may contain an mRNA encoding a replicase protein, and one or more RNA molecules capable of being replicated or amplified by the replicase.

### Inhibitory RNA

In some embodiments of all aspects of the disclosure, the nucleic acid is an inhibitory RNA.

The term “inhibitory RNA” as used herein means RNA which selectively hybridizes to and/or is specific for a target mRNA, thereby inhibiting (e.g., reducing) transcription and/or translation thereof. Inhibitory RNA includes RNA molecules having sequences in the antisense orientation relative to the target mRNA. Suitable inhibitory oligonucleotides typically vary in length from five to several hundred nucleotides, more typically about 20 to 70 nucleotides in length or shorter, even more typically about 10 to 30 nucleotides in length. Examples of inhibitory RNA include antisense RNA, ribozyme, iRNA, siRNA and miRNA. In some embodiments of all aspects of the disclosure, the inhibitory RNA is siRNA.

The term “antisense RNA” as used herein refers to an RNA which hybridizes under physiological conditions to DNA comprising a particular gene or to mRNA of said gene, thereby inhibiting transcription of said gene and/or translation of said mRNA. The size of the antisense RNA may vary from 15 nucleotides to 15,000, preferably 20 to 12,000, in particular 100 to 10,000, 150 to 8,000, 200 to 7,000, 250 to 6,000, 300 to 5,000 nucleotides, such as 15 to 2,000, 20 to 1,000, 25 to 800, 30 to 600, 35 to 500, 40 to 400, 45 to 300, 50 to 250, 55 to 200, 60 to 150, or 65 to 100 nucleotides.

By “small interfering RNA” or “siRNA” as used herein is meant an RNA molecule, preferably greater than 10 nucleotides in length, more preferably greater than 15 nucleotides in length, and most preferably 18, 19, 20, 21, 22, 23, 24, 25, 26, 27, 28, 29, or 30 nucleotides in length that is capable of binding specifically to a portion of a target mRNA. This binding induces a process, in which said portion of the target mRNA is cut or degraded and thereby the gene expression of said target mRNA inhibited. A range of 19 to 25 nucleotides is the most preferred size for siRNAs. Typically siRNAs comprise a single molecule in which two complementary portions are base-paired and are covalently linked by a single-stranded “hairpin” area. Without wishing to be bound by any theory, it is believed that the hairpin area of the siRNA molecule is cleaved intracellularly by the “Dicer” protein (or its equivalent) to form an siRNA of two individual base-paired RNA molecules.

As used herein, “target mRNA” refers to an RNA molecule that is a target for downregulation. In some embodiments, the target mRNA comprises an ORF encoding a pharmaceutically active peptide or polypeptide as specified herein. In some embodiments, the pharmaceutically active peptide or polypeptide is one whose expression (in particular increased expression, e.g., compared to the expression in a healthy subject) is associated with a disease. In some embodiments, the target mRNA comprises an ORF encoding a pharmaceutically active peptide or polypeptide whose expression (in particular increased expression, e.g., compared to the expression in a healthy subject) is associated with cancer.

According to the present disclosure, siRNA can be targeted to any stretch of approximately 19 to 25 contiguous nucleotides in any of the target mRNA sequences (the “target sequence”). Techniques for selecting target sequences for siRNA are given, for example, in Tuschl T. et al., “The siRNA User Guide”, revised Oct. 11, 2002, the entire disclosure of which is herein incorporated by reference. Further guidance with respect to the selection of target sequences and/or the design of siRNA can be found on the webpages of Protocol Online (www.protocol-online.com) using the keyword “siRNA”. Thus, in some embodiments, the sense strand of the siRNA used in the present disclosure comprises a nucleotide sequence substantially identical to any contiguous stretch of about 19 to about 25 nucleotides in the target mRNA.

siRNA can be obtained using a number of techniques known to those of skill in the art. For example, siRNA can be chemically synthesized or recombinantly produced. Preferably, siRNA is transcribed from recombinant circular or linear DNA plasmids using any suitable promoter. Selection of other suitable promoters is within the skill in the art. Selection of plasmids suitable for transcribing siRNA, methods for inserting nucleic acid sequences for expressing the siRNA into the plasmid, and IVT methods of in vitro transcription of said siRNA are within the skill in the art.

The term “miRNA” (microRNA) as used herein relates to non-coding RNAs which have a length of 21 to 25 (such as 21 to 23, preferably 22) nucleotides and which induce degradation and/or prevent translation of target mRNAs. miRNAs are typically found in plants, animals and some viruses, wherein they are encoded by eukaryotic nuclear DNA in plants and animals and by viral DNA (in viruses whose genome is based on DNA), respectively. miRNAs are post-transcriptional regulators that bind to complementary sequences on target messenger RNA transcripts (mRNAs), usually resulting in translational repression or target degradation and gene silencing.

miRNA can be obtained using a number of techniques known to those of skill in the art. For example, miRNA can be chemically synthesized or recombinantly produced using methods known in the art (e.g., by using commercially available kits such as the miRNA cDNA Synthesis Kit sold by Applied Biological Materials Inc.). Preferably, miRNA is transcribed from recombinant circular or linear DNA plasmids using any suitable promoter.

### DNA

In some embodiments of all aspects of the disclosure, the nucleic acid is DNA. Herein, the term “DNA” relates to a nucleic acid molecule which includes deoxyribonucleotide residues. DNA typically comprises the naturally occurring nucleic acids adenosine (dA), thymidine (dT), cytidine (dC) and guanosine (dG) (“d” represents “deoxy”). In preferred embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxy-ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof). DNA may be recombinant DNA and may be obtained by cloning of a nucleic acid, in particular cDNA. The cDNA may be obtained by reverse transcription of RNA.

### Pharmaceutically Active Peptides or Polypeptides

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Similarly, an RNA (such as mRNA) encodes a protein if translation of that RNA (e.g., in a cell) produces that protein.

In some embodiments, the active ingredient is an RNA (preferably mRNA), as described in the present disclosure, which comprises a nucleic acid sequence (e.g., an ORF) encoding one or more polypeptides, e.g., a peptide or protein, preferably a pharmaceutically active peptide or protein. In some embodiments, the RNA (preferably mRNA) described in the present disclosure is capable of expressing said peptide or protein, in particular if transferred into a cell or subject. Thus, in some embodiments, the RNA (preferably mRNA) described in the present disclosure contains a coding region (ORF) encoding a peptide or protein, preferably encoding a pharmaceutically active peptide or protein. In this respect, an “open reading frame” or “ORF” is a continuous stretch of codons beginning with a start codon and ending with a stop codon. Such RNA (preferably mRNA) encoding a pharmaceutically active peptide or protein is also referred to herein as “pharmaceutically active RNA” (or “pharmaceutically active mRNA”). In some embodiments, RNA (preferably mRNA) described in the present disclosure comprises a nucleic acid sequence encoding more than one peptide or polypeptide, e.g., two, three, four or more peptides or polypeptides. In some embodiments, RNA (preferably mRNA) described in the present disclosure comprises a nucleic acid sequence encoding one or more (e.g., 1, 2, 3, 4, 5, or more) patient-specific antigens suitable for personalized cancer therapy. In some embodiments, the lipid particle compositions comprising RNA may comprise one or more species of RNA, wherein each RNA encodes a different peptide or protein.

Preferably, the RNA (i) contains structural elements optimized for maximal efficacy of the RNA with respect to stability and translational efficiency (5′ cap, 5′ UTR, 3′ UTR, poly(A) sequence); (ii) is modified for optimized efficacy of the RNA (e.g., increased translation efficacy, decreased immunogenicity, and/or decreased cytotoxicity) (e.g., by replacing (partially or completely, preferably completely) naturally occurring nucleosides (in particular cytidine) with synthetic nucleosides (e.g., modified nucleosides selected from the group consisting of pseudouridine (ψ), N1-methyl-pseudouridine (m1ψ), and 5-methyl-uridine); and/or codon-optimization), or (iii) both (i) and (ii).

The term “pharmaceutically active peptide or protein” may be understood to mean a peptide or protein that can be used in the treatment of an individual where the expression of the peptide or protein would be of benefit, e.g., in ameliorating the symptoms of a disease or disorder. Preferably, a pharmaceutically active peptide or protein has curative or palliative properties and may be administered to ameliorate, relieve, alleviate, reverse, delay onset of or lessen the severity of one or more symptoms of a disease or disorder. A pharmaceutically active peptide or protein may have prophylactic properties and may be used to delay the onset of a disease or disorder or to lessen the severity of such disease or disorder.

Specific examples of pharmaceutically active peptides and proteins include, but are not limited to, cytokines, interferons, such as interferon-alpha (IFN-α), interferon beta (IFNβ) or interferon-gamma (IFN-γ), interleukins, such as interleukin 2 (IL2), IL-4, IL7, IL-10, IL-11, IL12, IL15, IL-21 and IL23, colony stimulating factors, such as colony stimulating factor (CSF), granulocyte colony stimulating factor (G-CSF), macrophage colony stimulating factor (M-CSF) and granulocyte-macrophage colony stimulating factor (GM-CSF), tumor necrosis factor (TNF), erythropoietin (EPO), and bone morphogenetic protein (BMP); immunoglobulin superfamily members including antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-δ, CD-3ε, CD79a, CD79b), co-stimulatory or inhibitory molecules (e.g., CD28, CD80, CD86); other immunologically active compounds such as tumor-associated antigens, pathogen-associated antigens (such as bacterial, parasitic, or viral antigens), allergens, and autoantigens.

### Aqueous Dispersion

The present disclosure provides an aqueous dispersion having an aqueous mobile phase and a dispersed phase. In this specification the term “dispersion” in its broadest sense takes its usual meaning in chemistry as a system in which distributed particles of one material (the “dispersed phase”) are dispersed in a phase of another material (the “continuous phase” or the “mobile phase”).

In one embodiment, the dispersion is a solid-liquid dispersion, in which the dispersed phase is solid and the mobile phase is a liquid. In one embodiment, the dispersion is a liquid-liquid dispersion, in which the dispersed phase and the mobile phase are both liquids.

In one embodiment, the dispersion is a colloid. The term “colloid” as used herein describes a stable mixture in which the dispersed particles do not settle out. Typically, the dispersed particles have at least in one direction a dimension roughly between 1 nm and 1 μm, or in such a system discontinuities are found at distances of that order.

In one embodiment, the dispersion is a suspension. The term “suspension” as used herein is a heterogeneous dispersion of larger particles in a medium. Unlike solutions and colloids, if left undisturbed for a long periods of time, the suspended particles may settle out of the mixture. The use of the terms “colloid” and “suspension” is sometimes overlapping or synonymous, with colloids in some instances being considered a sub-type of suspensions.

In one embodiment, the mobile phase is a solution. The term “solution” as used herein is a homogeneous mixture comprising a solvent which is typically water and solutes which can be salts, buffers, tonifiers and the like, as long as these materials are molecularly distributed within the solvent. The mobile phase may comprise solutes, as described further herein.

The dispersed phase comprises a lipid mixture including a cationic or cationically ionisable lipid, as defined herein. In one embodiment, the dispersed phase comprises a lipid mixture including a cationically ionisable lipid, as defined herein. In one embodiment, the dispersed phase comprises a lipid mixture comprising a cationically ionisable lipid, as defined herein, wherein the lipid mixture does not comprise a cationic lipid, as defined herein.

In one embodiment, the aqueous dispersion is substantially free of inorganic cations.

In one embodiment, the aqueous dispersion is substantially free of organic solvents.

In one embodiment, the aqueous dispersion is substantially free of nucleic acids. In one embodiment, the aqueous dispersion is substantially free of RNA.

In the present disclosure, it is preferred that the aqueous dispersion comprises pre-formed lipid nanoparticles (pre-LNPs). In the present disclosure, such pre-formed LNPs may be understood as oil-in-water emulsions in which the pre-LNP core materials are preferably in liquid state and hence have a melting point below body temperature. The pre-formed LNPs thus typically comprise a central complex disordered, non-lamellar phase made of lipid, but substantially free of nucleic acid. This is in contrast to the structure of a liposome which comprises unilamellar or multilamellar vesicular particles wherein the lamellae comprise lipid bilayers surrounding an encapsulated aqueous lumen. The lipids used for LNP formation typically do not form lamellar (bilayer) phases in water under physiological conditions. The LNPs typically do not comprise or encapsulate an aqueous core. The LNPs typically comprise a lipidic (or oily) core.

In some instances, the pre-LNPs described herein are not liposomes. In some instances, the pre-LNPs or the nucleic acid lipid particles formed from the pre-LNPs, described herein, are not lipoplexes.

The pre-formed LNPs are substantially free (as defined herein) of nucleic acids. The pre-formed LNPs may be free of nucleic acids, i.e., no nucleic acids are present in the pre-formed LNPs. Typically, no nucleic acids have been used or added in any of manufacturing steps in preparing the pre-formed LNPs. Pre-formed LNPs which are substantially free of nucleic acid can alternatively be described as “empty LNPs” and/or “loadable LNPs”, the step of loading the pre-LNPs with nucleic acid to produce loaded LNPs being as defined below.

In some embodiments, the pre-LNPs described herein have an average diameter that in some embodiments ranges from about 40 nm to about 1000 nm, from about 40 nm to about 800 nm, from about 40 nm to about 700 nm, from about 40 nm to about 600 nm, from about 40 nm to about 500 nm, from about 40 nm to about 450 nm, from about 40 nm to about 400 nm, from about 40 nm to about 350 nm, from about 40 nm to about 300 nm, from about 40 nm to about 250 nm, from about 40 nm to about 200 nm, from about 40 nm to about 150 nm, from about 40 nm to about 100 nm, from about 40 nm to about 90 nm, from about 40 nm to about 80 nm, from about 40 nm to about 70 nm. In some embodiments, pre-LNPs as described herein have an average diameter of less than 100 nm. In some embodiments, the pre-LNPs as described herein have an average diameter of from about 30 nm to about 100 nm. In some embodiments, the pre-LNPs as described herein have an average diameter of from about 40 nm to about 100 nm. In some embodiments, the pre-LNPs as described herein have an average diameter of from about 40 nm to about 70 nm.

In some instances, the aqueous dispersion comprises a dispersed phase comprising pre-LNPs having a size (i.e., a diameter) of from about 20 nm to about 500 nm, from about 20 nm to about 200 nm, from about 30 nm to about 100 nm, or preferably from about 60 nm to about 100 nm.

In one embodiment, the aqueous mobile phase comprises a cryoprotectant, as described in more detail below. This may be introduced in the mixing step or in a further processing step, as described in more detail below.

In one embodiment, the dispersed phase comprises a lipid mixture including a cationic or cationically ionisable lipid; and the aqueous mobile phase comprises an anion of an aqueous acid;
wherein the aqueous dispersion is substantially free of inorganic cations, organic solvents and RNA, and
wherein the aqueous mobile phase comprises a cryoprotectant.

In one embodiment, the dispersed phase comprises a cationic or cationically ionisable lipid; and the aqueous mobile phase comprises an anion of an aqueous acid;
wherein:
the concentration of the aqueous acid is at least 6 mM; and
the aqueous mobile phase is substantially free of inorganic cations, organic solvents and RNA.

In one embodiment, the dispersed phase comprises a cationic or cationically ionisable lipid; and the aqueous mobile phase comprises malate anion or a succinate anion; wherein the aqueous dispersion is substantially free of inorganic cations, organic solvents and RNA.

In one embodiment, the aqueous dispersion has a maximum pH of about 4.5, for example such as a maximum pH between 4.2 to 4.8. In one embodiment, the aqueous dispersion has a maximum pH of 4.5. The aqueous dispersion may have a pH of less than 4.5.

In one embodiment, the aqueous dispersion has a pH of from about 2.5 to about 4.5. The aqueous dispersion may have a pH of 2.5 to 4.5. The aqueous dispersion may have a pH of 2.5 to 3.5 or 3.5 to 4.5. The aqueous dispersion may have a pH of 4.0 to 4.5. The aqueous dispersion may have a pH of about 4.5.

The aqueous mobile phase comprises an anion of an aqueous acid. The acid may be any inorganic or organic acid which is at least partially miscible with water, and is capable of being at least partially deprotonated in water to produce the anion (i.e. the conjugate base) of the acid. It will therefore be understood by the skilled person that, depending on the pH and the strength of the acid, the aqueous mobile phase may contain both the undissociated acid and its corresponding anion in varying proportions. Strong acids are fully or largely deprotonated in water, so that the species in aqueous solution is mainly (in some embodiments completely) the anion of the acid. In contrast, weak acids are not fully deprotonated in water, so that the species in aqueous solution will comprise a mixture of undissociated acid and its conjugate base, the relative amounts of each depending on the pH.

In one embodiment, the anion is an acetate anion. In one embodiment, the anion is a malate anion. In one embodiment, the anion is a succinate anion.

Furthermore, the aqueous acid may undergo an acid-base reaction with a cationically ionisable lipid to produce the cationically ionisable lipid in its charged form and the acid in its anionic form. The extent to which such a reaction occurs depending on factors such as the basicity of the cationically ionisable lipid (when present in neutral form) and the pH.

Additionally, the anion of the aqueous acid may interact with the constitutively charged head group of a cationic lipid to form a lipid salt. It is expected that interactions between the anion of the aqueous acid and the cationic or cationically ionizable lipid will promote the formation of stable lipid particles. For example, formation of a lipid salt between the anion of the aqueous acid and a cationic lipid may affect the shape factor kappa (κ) (i.e., the volume ratio between the polar and apolar section of a lipid; κ=molecular volume (head, polar)/molecular volume (tail, apolar)) of the cationic lipid and promote formation of lipid nanoparticle structures (see, e.g., WO2008/043575, WO2009/047006, Siepi et al., Biophys J. 2011, 100, 2412-2421). For example, the lipid salt may have a shape factor κ of less than 0.25, optionally less than 0.15.

In one embodiment, the aqueous acid is an inorganic acid. Examples of suitable inorganic acids include hydrofluoric acid hydrochloric acid, hydrobromic acid, hydriodic acid, nitric acid, sulphuric acid and phosphoric acid.

In one embodiment, the aqueous acid is a water-soluble organic acid. Examples of suitable inorganic acids include sulfonic acids, carboxylic acids, dicarboxylic acids, hydroxy carboxylic acids (all as defined herein) or amino acids.

In one embodiment, the water-soluble organic acid is selected from the group consisting of acetic acid, malic acid, maleic acid, succinic acid, ascorbic acid, oxalic acid and citric acid, or combinations thereof. The water-soluble organic acid may be selected from the group consisting of acetic acid, malic acid, maleic acid, succinic acid, ascorbic acid, oxalic acid and citric acid. The water-soluble organic acid may be selected from the group consisting of acetic acid, malic acid, and succinic acid. The water-soluble organic acid may be selected from the group consisting of acetic acid, malic acid, maleic acid, succinic acid, ascorbic acid, and oxalic acid. The water-soluble organic acid may be selected from the group consisting of malic acid, maleic acid, succinic acid, ascorbic acid, and oxalic acid and citric acid. The water-soluble organic acid may be selected from the group consisting of malic acid, maleic acid, succinic acid, ascorbic acid, and oxalic acid.

In one embodiment, the water-soluble weak organic acid is acetic acid. In one embodiment, the water-soluble weak organic acid is malic acid. In one embodiment, the water-soluble weak organic acid is succinic acid.

In one embodiment, the aqueous mobile phase further comprises a cryoprotectant, as described and exemplified herein. In one embodiment, the aqueous dispersion comprises a cryoprotectant, as described and exemplified herein. In one embodiment, the cryoprotectant is a carbohydrate, such as a monosaccharide or disaccharide. In one embodiment, the cryoprotectant is selected from the group consisting of sucrose, trehalose and glucose, or a mixture of any thereof. Preferably, the cryoprotectant is sucrose.

In one embodiment, the cryoprotectant is sucrose or trehalose and is present in the aqueous dispersion at a concentration of about 1% to about 30% (w/v), about 2% to about 20% (w/v) or about 5% to about 15% (w/v). In one embodiment, the cryoprotectant is sucrose or trehalose and is present in a concentration of about 8% to about 12% (w/v), optionally about 10% (w/v). In one embodiment, the cryoprotectant is sucrose or trehalose and is present in a concentration of about 15% to about 25% (w/v), optionally about 18% to about 22% (w/v). In one embodiment, the cryoprotectant is sucrose or trehalose and is present in a concentration of about 20% (w/v). In one embodiment, the cryoprotectant is glucose and is present in the aqueous dispersion at a concentration of about 1% to about 15% (w/v), optionally about 2% to about 10% (w/v). In one embodiment, the cryoprotectant is glucose and is present in a concentration of about 4% to about 8% (w/v), optionally about 5% (w/v). In one embodiment, the cryoprotectant is glucose and is present in a concentration of about 8% to about 12% (w/v), optionally about 10% (w/v).

In one embodiment, the aqueous dispersion is substantially free of acetate buffers and citrate buffers. The aqueous dispersion may be substantially free of acetate buffers. The aqueous dispersion may be substantially free of citrate buffers. The aqueous mobile phase may be substantially free of citrate buffers. The aqueous dispersion and/or the aqueous mobile phase may be substantially free of a citrate buffer containing about 10 mM citrate, about 150 mM NaCl, pH of about 4.5. The aqueous dispersion may be substantially free of buffering agents. The aqueous dispersion may be substantially free of an acetate buffer, a citrate buffer, a phosphate buffer, and/or a tris buffer. The aqueous dispersion may be substantially free of a buffering agent selected from the group consisting of ammonium sulfate, sodium bicarbonate, sodium citrate, sodium acetate, potassium phosphate, and sodium phosphate. The aqueous dispersion may be substantially free of a buffering agent selected from the group consisting of ammonium sulfate, sodium bicarbonate, sodium citrate, sodium acetate, potassium phosphate, tris(hydroxymethyl)aminomethane (tris), sodium phosphate, and HEPES.

In one embodiment, the aqueous dispersion is substantially free of inorganic cations. Such inorganic cations are thought to affect the colloidal stability of the lipid dispersion and reduce the stability of the formulations. In one embodiment, the aqueous dispersion is substantially free (as defined herein) of alkali metal ions. In one embodiment the aqueous dispersion is substantially free of inorganic cations such as ammonium, sodium and/or potassium ions.

In one embodiment, the aqueous dispersion is substantially free (as defined herein) of organic solvents. In one embodiment, the term “substantially free of organic solvents” means that the aqueous dispersion contains less than about 5,000 ppm, such as less than about 4,000 ppm, such as less than about 3,000 ppm, such as less than about 2,000 ppm, such as less than about 1,000 ppm, such as less than about 900 ppm, such as less than about 800 ppm, such as less than about 700 ppm, such as less than about 600 ppm, such as less than about 500 ppm, such as less than about 400 ppm, such as less than about 300 ppm, such as less than about 200 ppm, such as less than about 100 ppm, by weight of the organic solvent, as a proportion of the total weight of the aqueous dispersion.

For example, the aqueous dispersion may be substantially free of water-soluble organic solvents, such as C1-4 alcohols (e.g. isopropanol or ethanol), ketones (e.g. acetone), or mixtures thereof, and/or apolar organic solvents, such as hydrocarbons such as pentane or hexane; chlorinated hydrocarbons such as dichloromethane or chloroform; or mixtures thereof. In one embodiment, the aqueous dispersion is substantially free of organic solvents including isopropanol, ethanol, and/or acetone.

In one embodiment, the concentration of the aqueous acid is at least 6 mM. In one embodiment, the concentration of the aqueous acid is in the range of 1 to 20 mM. In one embodiment, the concentration of the aqueous acid is in the range of 5.5 to 20 mM. In one embodiment, the concentration of the aqueous acid is in the range of 6 to 20 mM. In one embodiment, the concentration of the aqueous acid is in the range of 2.5 to 10 mM. In one embodiment, the concentration of the aqueous acid is in the range of 5.5 to 10 mM. In one embodiment, the concentration of the aqueous acid is in the range of 6 to 10 mM. It will be understood in this context that this concentration includes both the undissociated acid and its conjugate base.

In one embodiment, the cationic or cationically ionisable lipid and the anions of aqueous acid are present in a molar ratio of between about 20:1 and about 1:20. In one embodiment, the cationic or cationically ionisable lipid and the anions of aqueous acid are present in a molar ratio of between about 10:1 and about 1:10. In one embodiment, the cationic or cationically ionisable lipid and the anions of aqueous acid are present in a molar ratio of between about 5:1 and about 1:5. In one embodiment, the cationic or cationically ionisable lipid and the anions of aqueous acid are present in a molar ratio of between about 3:1 and about 1:3. It will be understood in this context that the moles of cationic or cationically ionisable lipid includes both the un-ionised lipid and its conjugate acid, and the moles of aqueous acid includes both the undissociated acid and its conjugate base.

When the acid is a strong acid, in one embodiment, the cationic or cationically ionisable lipid and the anions of aqueous acid are present in a molar ratio of between 1:10 and 10:1. In one embodiment, the cationic or cationically ionisable lipid and the anions of aqueous acid are present in a molar ratio of between 1:5 and 5:1. In one embodiment, the cationic or cationically ionisable lipid and the anions of aqueous acid are present in a molar ratio of between 1:3 and 3:1. In one embodiment, the cationic or cationically ionisable lipid and the anions of aqueous acid are present in a molar ratio of between 1:2 and 2:1. In one embodiment, the cationic or cationically ionisable lipid and the anions of aqueous acid are present in a molar ratio of between 1:1.5 and 1.5:1. In one embodiment, the cationic or cationically ionisable lipid and the anions of aqueous acid are present in a molar ratio of between 1:1.2 and 1.2:1. In one embodiment, the cationic or cationically ionisable lipid and the anions of aqueous acid are present in a molar ratio of 1:1.

When the acid is a weak acid, in one embodiment, the cationic or cationically ionisable lipid and the anions of aqueous acid are present in a molar ratio of between 1:20 and 5:1. In one embodiment, the cationic or cationically ionisable lipid and the anions of aqueous acid are present in a molar ratio of between 1:10 and 2.5:1. In one embodiment, the cationic or cationically ionisable lipid and the anions of aqueous acid are present in a molar ratio of between 1:6 and 1.5:1. In one embodiment, the cationic or cationically ionisable lipid and the anions of aqueous acid are present in a molar ratio of between 1:4 and 1.25:1. In one embodiment, the cationic or cationically ionisable lipid and the anions of aqueous acid are present in a molar ratio of between 1:3 and 1:1.33. In one embodiment, the cationic or cationically ionisable lipid and the anions of aqueous acid are present in a molar ratio of 1:2.

In some instances, the aqueous dispersion comprises a dispersed phase comprising lipid particles. In some instances, the lipid particles of the dispersed phase are lipid nanoparticles. In some instances, the lipid particles of the dispersed phase are not liposomes. In some instances, the aqueous dispersion comprises a dispersed phase comprising lipid particles having a size (i.e., a diameter) of from about 20 nm to about 500 nm, from about 20 nm to about 200 nm, from about 30 nm to about 180 nm, from about 40 nm to about 120 nm, or preferably from about 40 nm to about 80 nm. In some instances, the aqueous dispersion comprises a dispersed phase comprising lipid particles having a size (i.e., a diameter) of not more than about 200 nm.

### Storage Matrix—Cryoprotectants and Other Ingredients

In one embodiment, the aqueous dispersion (typically containing pre-LNPs) also contains a storage matrix. In this specification, the term “storage matrix” when used in its broadest sense typically covers any substance typically used to aid storage and improve the shelf-life of the aqueous dispersion. The storage matrix is typically added to the aqueous dispersion after the filtration (e.g., TFF)/dialysis step.

In one embodiment, the storage matrix comprises a cryoprotectant. In this specification, the term “cryoprotectant” when used in its broadest sense means any substance capable of protecting a composition from damage caused by freezing and/or by ice formation. Examples of cryoprotectants include glycols (i.e. alcohols containing at least two hydroxy groups, such as glycerol and propylene glycol) and carbohydrates, as defined and exemplified herein.

In one embodiment, the cryoprotectant is a carbohydrate. In one embodiment, the cryoprotectant is a monosaccharide or disaccharide. In one embodiment, the cryoprotectant is selected from the group consisting of sucrose, trehalose, lactose and glucose, or a mixture of any thereof. In one embodiment, the cryoprotectant is selected from the group consisting of sucrose, trehalose and glucose, or a mixture of any thereof. Preferably, the cryoprotectant is sucrose.

When the aqueous dispersion also contains a storage matrix which is a carbohydrate, typically, this is present in a concentration of about 1% to about 30% (w/v). In one embodiment, the storage matrix is a carbohydrate and is present in a concentration of about 2% to about 20% (w/v). In one embodiment, the storage matrix is a carbohydrate and is present in a concentration of about 5% to about 15% (w/v). In one embodiment, the storage matrix is a carbohydrate and is present in a concentration of about 8% to about 12% (w/v). In one embodiment, the storage matrix is a carbohydrate and is present in a concentration of about 10% (w/v).

In one embodiment, the storage matrix is sucrose or trehalose and is present in a concentration of about 1% to about 30% (w/v). In one embodiment, the storage matrix is sucrose or trehalose and is present in a concentration of about 2% to about 20% (w/v). In one embodiment, the storage matrix is sucrose or trehalose and is present in a concentration of about 5% to about 15% (w/v). In one embodiment, the storage matrix is sucrose or trehalose and is present in a concentration of about 8% to about 12% (w/v). In one embodiment, the storage matrix is sucrose or trehalose and is present in a concentration of about 10% (w/v). In one embodiment, the storage matrix is sucrose or trehalose and is present in a concentration of about 15% to about 25% (w/v). In one embodiment, the storage matrix is sucrose or trehalose and is present in a concentration of about 18% to about 22% (w/v). In one embodiment, the storage matrix is sucrose or trehalose and is present in a concentration of about 20% (w/v).

In one embodiment, the storage matrix is glucose and is present in a concentration of about 1% to about 15% (w/v). In one embodiment, the storage matrix is glucose and is present in a concentration of about 2% to about 10% (w/v). In one embodiment, the storage matrix is glucose and is present in a concentration of about 4% to about 8% (w/v). In one embodiment, the storage matrix is glucose and is present in a concentration of about 5% (w/v). In one embodiment, the storage matrix is glucose and is present in a concentration of about 8% to about 12% (w/v). In one embodiment, the storage matrix is glucose and is present in a concentration of about 10% (w/v).

### Method of Forming Aqueous Dispersion

In a further aspect, the present disclosure provides methods for producing the aqueous dispersion of the invention.

A number of methods of making dispersions are known in the art and the skilled person would be readily capable of selecting a suitable method and applying this to make the dispersion compositions of the present invention.

In one embodiment, the method comprises mixing:
(i) a lipid mixture comprising a cationic or cationically ionisable lipid;
(ii) an aqueous phase comprising an aqueous acid and a cryoprotectant;
to produce the aqueous dispersion comprising an anion of the aqueous acid.

In one embodiment, the method comprises:
(a) mixing:
(i) a lipid mixture comprising a cationic or cationically ionisable lipid; and
(ii) an aqueous phase comprising an aqueous acid;
to produce a first intermediate aqueous dispersion comprising an anion of the aqueous acid; and
(b) adding the cryoprotectant to the first intermediate aqueous dispersion to produce the aqueous dispersion.

In one embodiment, the method comprises:
(a) mixing:
(i) a lipid mixture comprising a cationic or cationically ionisable lipid dissolved in a water-soluble organic solvent; and
(ii) an aqueous phase;
the lipid mixture and/or the aqueous phase comprising the aqueous acid;
to produce a first intermediate acidified aqueous lipid dispersion comprising an anion of the aqueous acid;
(b) performing on the first intermediate acidified aqueous lipid dispersion a dialysis or filtration step at a pH of about 2.5 to about 5.5, to remove the organic solvent and produce a second intermediate aqueous dispersion; and
(c) adding a cryoprotectant to the second intermediate aqueous dispersion; to produce the aqueous dispersion comprising an anion of the aqueous acid. The addition of the cryoprotectant typically does not affect the pH, such that the pH of the aqueous dispersion comprising an anion of the aqueous acid is essentially the same as that of the second intermediate aqueous dispersion, i.e., about 2.5 to about 5.5.

In one embodiment, the method comprises:
i) mixing a lipid mixture comprising a cationic or cationically ionisable lipid dissolved in a water-soluble organic solvent with an aqueous phase, wherein the lipid solution and/or the aqueous phase comprises an aqueous acid, to produce a first intermediate acidified aqueous lipid dispersion comprising an anion of the aqueous acid;
ii) performing on the first intermediate acidified aqueous lipid dispersion a dialysis or filtration step at a pH of about 2.5 to about 5.5, or at a pH of about 6.5 to about 8.5, to remove the organic solvent and produce a second intermediate aqueous dispersion; and
iii) adding a cryoprotectant to the second intermediate aqueous dispersion; to produce the aqueous dispersion;
wherein the aqueous dispersion is substantially free of inorganic cations, organic solvents and RNA. The addition of the cryoprotectant typically does not affect the pH, such that the pH of the aqueous dispersion is essentially the same as that of the second intermediate aqueous dispersion, i.e., about 2.5 to about 5.5 or about 6.5 to about 8.5.

In one embodiment, the method comprises:
i) preparing a solution of a lipid mixture comprising a cationic or cationically ionisable lipid dissolved in water-soluble and/or apolar organic solvents (preferably wherein such solvents are volatile);
ii) evaporation of the organic solvent below atmospheric pressure, to provide the lipid mixture in the form of a film (or layer) of lipids, optionally a thin film, typically a homogenous thin film;
iii) addition of an aqueous acid to the film (e.g., thin film) of lipid mixture, to produce the aqueous dispersion; and
iv) diluting the aqueous dispersion with a cryoprotectant;
wherein the aqueous dispersion is substantially free of inorganic cations, organic solvents and RNA. An exemplary suitable solvent for dissolving the lipid mixture in step i) may be, for example, a 1:1 mixture of methanol and dichloromethane.

This method is referred to herein as “the thin film method”.

In one embodiment of the above thin film method, the method further comprises the following step ii′) after step ii):
ii′) reduction of the particle size of the aqueous dispersion by standard unit operations such as extrusion, sonication, homogenization, preferably by pore size extrusion.

In one embodiment, the method comprises:
i) preparing a solution of a lipid mixture comprising a cationic or cationically ionisable lipid dissolved in a non-polar water immiscible organic solvent, or if required for lipid solubility a mixture of a non-polar water immiscible organic solvent and a polar organic solvent;
ii) adding an aqueous phase to produce a first intermediate composition including the lipid mixture,
wherein the solution of the lipid mixture and/or the aqueous phase are acidified;
iv) removal of the organic solvents by standard unit operations, such as evaporation or filtration (preferably by evaporation), below atmospheric pressure, to produce a second intermediate composition including the lipid mixture;
v) sonicating the second intermediate composition, to produce the aqueous dispersion; and
vi) diluting the aqueous dispersion with a cryoprotectant;
wherein the aqueous dispersion is substantially free of inorganic cations, organic solvents and RNA.

This method is referred to herein as “the emulsification method”.

In one embodiment, the emulsification method further comprises the following step v′) after step v):
v′) reduction of the particle size of the aqueous dispersion by standard unit operations such as extrusion, sonication, homogenization, preferably pore size extrusion.

In one embodiment, the method further comprises storing the aqueous dispersion at a pH of between 2.5 and 5.5. In one embodiment, the method further comprises storing the aqueous dispersion at a pH of between 3.0 and 5.5. In one embodiment, the method further comprises storing the aqueous dispersion at a pH of between 3.5 and 5.0. In one embodiment, the method further comprises storing the aqueous dispersion at a pH of between 3.5 and 4.5.

In one embodiment, the methods of the invention for producing the aqueous dispersion are performed at from about 0° C. to about 25° C., optionally from about 4° C. to about 25° C., preferably from about 15° C. to about 25° C. In one embodiment, the methods of the invention for producing the aqueous dispersion are performed at about room temperature (e.g., 18-25° C.).

### Mixing Step

The mixing step of the method of forming the aqueous dispersion of the present invention comprises mixing an organic phase comprising a lipid mixture comprising (i) a cationically ionisable lipid dissolved in a water-soluble organic solvent; and (ii) an aqueous phase, the aqueous phase comprising an anion of an aqueous acid, wherein the aqueous dispersion is substantially free of inorganic cations, organic solvents and RNA.

In the methods of the invention, the organic solvent (e.g., the water-soluble organic solvent) may be selected from the lists of Class 2 and Class 3 solvents, as described in the FDA's “Q3C—Tables and List Guidance for Industry”, June 2017, Revision 3 (see, e.g., https://www.fda.gov/media/71737/download). When the organic solvent is a water-soluble organic solvent, examples include C1-4 alcohols (e.g. isopropanol or ethanol), ketones (e.g. acetone), or mixtures thereof. When the organic solvent is an apolar organic solvent, examples include hydrocarbons such as pentane or hexane; chlorinated hydrocarbons such as dichloromethane or chloroform; or mixtures thereof. The organic solvent (e.g., the water-soluble organic solvent) is preferably ethanol or isopropanol.

In one embodiment, the lipid mixture does not comprise phosphatidylserine.

In a preferred embodiment, the acid is a water-soluble organic acid, as defined generally above. Examples of suitable organic acids include sulfonic acids, phosphoric acids, phosphonic acids, carboxylic acids, dicarboxylic acids, or hydroxy carboxylic acids (all as defined herein).

In one embodiment, the water-soluble organic acid is selected from the group consisting of acetic acid, malic acid, succinic acid, and citric acid, or combinations thereof. In one embodiment, the water-soluble organic acid may be selected from the group consisting of acetic acid and malic acid, or combinations thereof.

In one embodiment, the water-soluble organic acid is acetic acid. In one embodiment, the water-soluble organic acid is malic acid. In one embodiment, the water-soluble organic acid is succinic acid. In one embodiment, the water-soluble organic acid is citric acid.

In one embodiment, the concentration of the acid is in the range of about 0.1 to about 20 mM. In one embodiment, the concentration of the acid is in the range of about 0.2 to about 15 mM. In one embodiment, the concentration of the acid is in the range of about 0.5 to about 10 mM. In one embodiment, the concentration of the acid is in the range of about 1 to about 5 mM. In one embodiment, the concentration of the acid is in the range of about 2 to about 10 mM. In one embodiment, the concentration of the acid is in the range of about 0.5 to about 5 mM. In one embodiment, the concentration of the acid is in the range of about 3 to about 15 mM. In one embodiment, the concentration of the acid is in the range of about 5 to about 8 mM. In one embodiment, the concentration of the acid is in the range of about 8 to about 12 mM. It will be understood in this context that this concentration includes both the undissociated acid and its conjugate base.

In one embodiment, the acid is acetic acid and is present in a concentration in the range of about 0.2 to about 20 mM. In one embodiment, the acid is acetic acid and is present in a concentration in the range of about 0.5 to about 10 mM. In one embodiment, the acid is acetic acid and is present in a concentration in the range of about 0.2 to about 3 mM. In one embodiment, the acid is acetic acid and is present in a concentration in the range of about 0.5 to about 2 mM. In one embodiment, the acid is acetic acid and is present in a concentration in the range of about 1 to about 1.5 mM. In one embodiment, the acid is acetic acid and is present in a concentration of about 1.25 mM. In one embodiment, the acid is acetic acid and is present in a concentration in the range of about 0.5 to about 4 mM. In one embodiment, the acid is acetic acid and is present in a concentration in the range of about 1 to about 3.5 mM. In one embodiment, the acid is acetic acid and is present in a concentration in the range of about 2 to about 3 mM. In one embodiment, the acid is acetic acid and is present in a concentration of about 2.5 mM. In one embodiment, the acid is acetic acid and is present in a concentration in the range of about 1 to about 8 mM. In one embodiment, the acid is acetic acid and is present in a concentration in the range of about 1 to about 8 mM. In one embodiment, the acid is acetic acid and is present in a concentration in the range of about 2 to about 7 mM. In one embodiment, the acid is acetic acid and is present in a concentration in the range of about 4 to about 6 mM. In one embodiment, the acid is acetic acid and is present in a concentration in the range of about 4.5 to about 5.5 mM. In one embodiment, the acid is acetic acid and is present in a concentration of about 5 mM.

In one embodiment, the acid is malic acid and is present in a concentration in the range of about 0.1 to about 5 mM. In one embodiment, the acid is malic acid and is present in a concentration in the range of about 0.4 to about 4 mM. In one embodiment, the acid is malic acid and is present in a concentration in the range of about 0.8 to about 2 mM. In one embodiment, the acid is malic acid and is present in a concentration in the range of about 1 to about 1.5 mM. In one embodiment, the acid is malic acid and is present in a concentration of about 1.25 mM. In one embodiment, the acid is malic acid and is present in a concentration in the range of about 0.5 to about 4 mM. In one embodiment, the acid is malic acid and is present in a concentration in the range of about 1 to about 3.5 mM. In one embodiment, the acid is malic acid and is present in a concentration in the range of about 2 to about 3 mM. In one embodiment, the acid is malic acid and is present in a concentration of about 2.5 mM. In one embodiment, the acid is malic acid and is present in a concentration in the range of about 1 to about 8 mM. In one embodiment, the acid is malic acid and is present in a concentration in the range of about 1 to about 8 mM. In one embodiment, the acid is malic acid and is present in a concentration in the range of about 2 to about 7 mM. In one embodiment, the acid is malic acid and is present in a concentration in the range of about 4 to about 6 mM. In one embodiment, the acid is malic acid and is present in a concentration in the range of about 4.5 to about 5.5 mM. In one embodiment, the acid is malic acid and is present in a concentration of about 5 mM.

In one embodiment, the acid is citric acid and the concentration of the acid is greater than 0.3 mM. In one embodiment, the acid is citric acid and is present in a concentration in the range of about 0.2 to about 15 mM. In one embodiment, the acid is citric acid and is present in a concentration in the range of about 0.5 to about 10 mM. In one embodiment, the acid is citric acid and is present in a concentration in the range of about 1 to about 8 mM. In one embodiment, the acid is citric acid and is present in a concentration in the range of about 2 to about 7 mM. In one embodiment, the acid is citric acid and is present in a concentration in the range of about 4 to about 6 mM. In one embodiment, the acid is citric acid and is present in a concentration in the range of about 4.5 to about 5.5 mM. In one embodiment, the acid is citric acid and is present in a concentration of about 5 mM.

In one embodiment, the acid is succinic acid and is present in a concentration in the range of about 0.2 to about 10 mM. In one embodiment, the acid is succinic acid and is present in a concentration in the range of about 0.4 to about 5 mM. In one embodiment, the acid is succinic acid and is present in a concentration in the range of about 1 to about 3.5 mM. In one embodiment, the acid is succinic acid and is present in a concentration in the range of about 2 to about 3 mM. In one embodiment, the acid is succinic acid and is present in a concentration of about 2.5 mM.

In one embodiment, the mixing is carried out using a T-mixer or Y-mixer.

In one embodiment, the flow rate during mixing is at least 50 mL/min. The flow rate during mixing may be from about 50 mL/min to about 400 mL/min, optionally from about 100 mL/min to about 300 mL/min, optionally from about 150 mL/min to about 250 mL/min. The volume ratio of organic solvent to aqueous phase may be from about 1:6 to about 6:1, optionally from about 1:2 to about 1:6, optionally about 1:4.

The pH of the aqueous dispersion as produced according to any of the above methods may be about 2.5 to about 5.5, optionally about 2.5 to about 4.5. The pH of the aqueous dispersion as produced according to any of the above methods may be about 2.5 to about 3.5. The pH of the aqueous dispersion as produced according to any of the above methods may be about 3.5 to about 4.5. The pH of the aqueous dispersion as produced according to any of the above methods may be about 6.5 to about 8.5, optionally 6.8 to 8.5, further optionally about 7.0 to about 8.0.

### Cryoprotectant in Mixing Step

In one embodiment, the aqueous phase further contains a cryoprotectant, as defined and exemplified herein. In this embodiment, the aqueous phase therefore contains an anion of an aqueous acid, as defined and exemplified above, and a cryoprotectant, as defined and exemplified above.

In one embodiment, the cryoprotectant is a carbohydrate. In one embodiment, the cryoprotectant is a monosaccharide or disaccharide. In one embodiment, the cryoprotectant is selected from the group consisting of sucrose, trehalose, lactose and glucose, or a mixture of any thereof. In one embodiment, the cryoprotectant is selected from the group consisting of sucrose, trehalose and glucose, or a mixture of any thereof. Preferably, the cryoprotectant is sucrose.

When the aqueous phase also contains a cryoprotectant which is a carbohydrate, typically, this is present in a concentration of about 1% to about 50% (w/v).

In one embodiment, the cryoprotectant is selected from the group consisting of sucrose, trehalose and lactose, and is present in a concentration of about 2% to about 20% (w/v). In one embodiment, the cryoprotectant is selected from the group consisting of sucrose, trehalose and lactose, and is present in a concentration of about 3% to about 25% (w/v). In one embodiment, the cryoprotectant is selected from the group consisting of sucrose, trehalose and lactose, and is present in a concentration of about 5% to about 20% (w/v). In one embodiment, the cryoprotectant is selected from the group consisting of sucrose, trehalose and lactose, and is present in a concentration of about 8% to about 12% (w/v). In one embodiment, the cryoprotectant is selected from the group consisting of sucrose, trehalose and lactose, and is present in a concentration of about 18% to about 22% (w/v).

In one embodiment, the cryoprotectant is glucose and is present in a concentration of about 1% to about 15% (w/v). In one embodiment, the cryoprotectant is glucose and is present in a concentration of about 3% to about 12% (w/v). In one embodiment, the cryoprotectant is glucose and is present in a concentration of about 5% to about 10% (w/v).

### Further Processing Steps

In one embodiment, the method further comprises subjecting the aqueous dispersion to one or more further processing steps. In one embodiment, the method further comprises subjecting the aqueous dispersion to one or more further dilution or purification steps.

### Dialysis/Filtration Step

In one embodiment, the purification steps comprise a dialysis or filtration step, the purpose of which is typically to remove the organic solvent. In one embodiment, the dialysis or filtration step is performed at a pH of about 4.0 to about 5.0. In one embodiment, the dialysis or filtration step comprise tangential flow filtration.

In one embodiment the dialysis or filtration step employs a composition comprising a compound selected from any of the following classes (a) to (d):
(a) an amino acid or a mixture thereof, preferably:
(i) an acidic amino acid, preferably selected from the group consisting of aspartic acid glutamic acid, 3-hydroxyglutamic acid, and alpha-aminoadipic acid; or a mixture thereof;
(ii) a basic amino acid, preferably selected from the group consisting of arginine, histidine, and lysine, or a mixture thereof;
or a mixture of (i) and (ii), or a mixture of either or both (i) and (ii) with a neutral amino acid;
(b) an organic acid, preferably selected from the group consisting of acetic acid, malic acid, succinic acid, citric acid, and methyl malonic acid; or a mixture thereof,
(c) a cryoprotectant, optionally wherein the cryoprotectant is a carbohydrate, such as a monosaccharide or disaccharide, preferably wherein the cryoprotectant is selected from the group consisting of sucrose, trehalose, lactose and glucose, or a mixture of any thereof;
or a mixture of any thereof.

In one embodiment, the dialysis or filtration step is carried out using one or more water-soluble weak organic acids. In one embodiment, the water-soluble weak organic acid is selected from the group consisting of acetic acid, malic acid, maleic acid and succinic acid. In one embodiment, the water-soluble weak organic acid is acetic acid.

In one embodiment, the dialysis or filtration step is carried out using an amino acid. In one embodiment, the composition used for dialysis or filtration is an acidic amino acid, as defined and exemplified above, or a mixture thereof. In one embodiment, the composition used for dialysis or filtration is selected from the group consisting of aspartic acid, glutamic acid, 3-hydroxyglutamic acid, and alpha-aminoadipic acid, or a mixture thereof.

In one embodiment, the composition used for dialysis or filtration is a mixture of an amino acid, as defined and exemplified above, or a mixture thereof, and a water-soluble organic acid, as defined and exemplified above, or a mixture thereof.

### Dilution/Addition of Storage Matrix Cryoprotectant

In one embodiment, the method may further comprise the additional step of adding a storage matrix to the aqueous dispersion. This method preferably takes place after the dialysis or filtration step. However, in an alternative, it may take place immediately after the mixing step to form the aqueous dispersion.

The storage matrix used in this step may be any of those defined and exemplified above. In one embodiment, the storage matrix comprises a cryoprotectant, such that. the dilution steps comprise addition of cryoprotectant. The cryoprotectant dilutes the aqueous dispersion and protects the pre-LNPs from damage due to freezing. The cryoprotectant is not especially limited provided it is capable of performing this function. In one embodiment, the cryoprotectant is selected from the group consisting of sucrose, trehalose, glucose, sorbitol, fructose, maltose, xylose and dextran, or a mixture of any thereof. In one embodiment, the cryoprotectant is selected from the group consisting of sucrose, glycerol, trehalose, lactose, glucose and mannitol. In a preferred embodiment, the cryoprotectant is selected from the group consisting of sucrose, trehalose, and glucose, or a mixture of any thereof. In a more preferred embodiment, the cryoprotectant is selected from the group consisting of sucrose and trehalose, or a mixture thereof. In one embodiment, the cryoprotectant is sucrose.

In one embodiment, the storage matrix further comprises a compound selected from the following classes (a) to (c):
(a) an amino acid, as defined and exemplified above, such as
(i) an acidic amino acid, as defined and exemplified above, preferably selected from the group consisting of aspartic acid, glutamic acid, 3-hydroxyglutamic acid, and alpha-aminoadipic acid, or a mixture thereof;
(ii) a basic amino acid, as defined and exemplified above, preferably selected from the group consisting of arginine, histidine, and lysine; or a mixture thereof; or a mixture of (i) and (ii), optionally mixed with a neutral amino acid;
(b) an organic acid, as defined and exemplified above, preferably selected from the group consisting of acetic acid, malic acid, succinic acid, citric acid, and methyl malonic acid, or a mixture thereof, or a mixture of any thereof.

In one embodiment, the method further comprises adding peptide-conjugated lipid (as further described herein) to the lipid particles comprised in the dispersed phase of the aqueous dispersion. In some instances, the peptide-conjugated lipid may displace (i.e., replace) a corresponding portion of the steroid (e.g., cholesterol) in the lipid particles comprised in the dispersed phase of the aqueous dispersion.

Thus, the composition of the lipid particles comprised in the dispersed phase of the aqueous dispersion before addition of peptide-conjugated lipid may comprise a cationic or cationically ionizable lipid as described herein, a neutral or zwitterionic phospholipid as descried herein, a steroid as described herein; and optionally a grafted lipid as described herein, in a molar ratio of 20-70 mol %:5-15 mol %:20-60 mol % and optionally 0.5-10 mol %, respectively; preferably 40-60 mol %:8-12 mol %:30-50 mol % and optionally 1.0-5 mol %, respectively. Following addition of the peptide-conjugated lipid, the peptide-conjugated lipid may comprise 0.05-1.0 mol %, optionally 0.1 to 0.5 mol %, preferably 0.1-0.3 mol % of the lipid particles comprised in the dispersed phase of the aqueous dispersion, with a corresponding reduction in the mol % of the steroid.

In one embodiment, the purification is carried out using aqueous phase essentially free of buffering agents. In one embodiment, the purification is carried out using aqueous phase essentially free of buffering agents, other than amino acids.

### Further Optional Steps

In one embodiment, the method further comprises the step of drying of the aqueous dispersion. In one embodiment, the drying is lyophilisation (freeze drying). In one embodiment, the drying is spray drying.

In one embodiment, the purification comprises sterile filtration of the aqueous dispersion. Typically, the sterile filtration uses a 0.22 μm filter. In one embodiment, the filter is a polyethersulfone (PES) filter.

In one embodiment, the method further comprises storing the aqueous dispersion for 24 hours, 48 hours, 72 hours, 5 days, 1 week, 2 weeks, 4 weeks, 2 months, 4 months, 6 months, 9 months, 12 months, 18 months, 2 years, 3 years, or more. The aqueous dispersion may be stored at about 25° C., at about room temperature (e.g., 18-23° C.), at about 4-8° C., at about 4° C., at about −20° C., or at about −80° C. The aqueous dispersion may be stored at about 4° C. or at about −20° C. In one embodiment, the method further comprises the step of freezing the aqueous dispersion, for example at a temperature between −15° C. to −90° C., preferably at a temperature of from about −18° to about −25° C. In one embodiment, the method further comprising the step of drying of the aqueous dispersion. In one embodiment, the drying is freeze drying or spray drying.

In one embodiment, the aqueous dispersion is stable for at least 3 months at 4° C. In one embodiment, the aqueous dispersion is stable for at least 6 months at −20° C. Therefore, in one embodiment, there is provided an aqueous dispersion which is stable for at least 3 months at 4° C. In one embodiment, there is provided an aqueous dispersion which is stable for at least 6 months at −20° C. In this context “stable” may be understood to mean that the size (Z_{average}) and/or size distribution and/or PDI of the particles in the aqueous dispersion after storage for the indicated time period at the indicated temperature is essentially equal to the size (Z_{average}) and/or size distribution and/or PDI of the particles before storage and immediately after preparation. For example, the size (Z_{average}) and/or size distribution and/or PDI of the particles in the aqueous dispersion may not change by more than 20%, optionally by more than 10%, preferably by more than 5%, during the indicated storage.

### Nucleic Acid-Lipid Particle

The present disclosure further provides a lipid particle comprising a lipid or lipid mixture, as defined herein, and a nucleic acid. In one embodiment, there is provided a lipid particle obtained or obtainable by the methods defined herein. Such particles are also referred to herein as “nucleic acid-lipid particles”. When the nucleic acid is RNA, such particles are also referred to herein as “RNA-lipid particles”.

In one embodiment, the nucleic acid is RNA. In one embodiment, the nucleic acid is mRNA, saRNA, taRNA, or mixtures thereof. In one embodiment, the nucleic acid is mRNA. In one embodiment, the nucleic acid is DNA. In one embodiment, the nucleic acid is RNA which encodes for one or more personalized cancer antigens.

In the present disclosure, it is preferred that the nucleic acid-lipid particle is a lipid nanoparticle (LNP). The function of the LNP is to stabilise and encapsulate the nucleic acid to enable it to be delivered into a cell while facilitating its uptake into the cell and release into the cytosol. The LNPs and/or their lipid components may have adjuvant activity.

In the present disclosure, LNPs may be understood as oil-in-water emulsions in which the LNP core materials are preferably in liquid state and hence have a melting point below body temperature. LNPs thus typically comprise a central complex of mRNA and lipid embedded in a disordered, non-lamellar phase made of lipid. This is in contrast to the structure of a liposome which comprises unilamellar or multilamellar vesicular particles wherein the lamellae comprise lipid bilayers surrounding an encapsulated aqueous lumen. In some instances, the nucleic acid-lipid particles described herein are not liposomes. In some instances, the nucleic acid-lipid particles described herein are not lipoplexes.

Lipid nanoparticles (LNP) are obtainable from combining a nucleic acid with lipids. The lipids used for LNP formation typically do not form lamellar (bilayer) phases in water under physiological conditions. The LNPs typically do not comprise or encapsulate an aqueous core. The LNPs typically comprise a lipidic (or oily) core.

In some embodiments, the lipid nanoparticles described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm. In some embodiments, the lipid nanoparticles described herein have an average diameter that in some embodiments ranges from about 60 nm to about 100 nm.

In one embodiment, the nucleic acid-lipid particles are stable for at least 3 months at 4° C. In one embodiment, the nucleic acid-lipid particles are stable for at least 6 months at −20° C.

In one embodiment, the nucleic acid-lipid particles are present in a composition having a pH of between 4.0 and 6.5. In one embodiment, the nucleic acid-lipid particles are present in a composition having a pH of between 4.5 and 6.0. In one embodiment, the nucleic acid-lipid particles are present in a composition having a pH of between 4.6 and 5.8. In one embodiment, the nucleic acid-lipid particles are present in a composition having a pH of between 5.0 and 5.5. In one embodiment, the nucleic acid-lipid particles are present in a composition having a pH of about 5.1. In one embodiment, the nucleic acid-lipid particles are present in a composition having a pH of about 5.2. In one embodiment, the nucleic acid-lipid particles are present in a composition having a pH of about 5.3. In one embodiment, the nucleic acid-lipid particles are present in a composition having a pH of about 5.4.

In one embodiment, the nucleic acid-lipid particles are present in a composition having a pH of between 7.0 and 9.0. In one embodiment, the nucleic acid-lipid particles are present in a composition having a pH of between 7.0 and 8.5. In one embodiment, the nucleic acid-lipid particles are present in a composition having a pH of between 7.5 and 8.1. In one embodiment, the nucleic acid-lipid particles are present in a composition having a pH of about 7.8. In one embodiment, the nucleic acid-lipid particles are present in a composition having a pH of about 7.5.

Therefore, in one embodiment, there is provided a nucleic acid-lipid particle which is stable for at least 3 months at 4° C. In one embodiment, there is provided a nucleic acid-lipid particle which is stable for at least 6 months at −20° C.

In one embodiment, the integrity of the nucleic acid (preferably RNA) in the nucleic acid-lipid particles does not decrease by more than 20% after storage of the nucleic acid-lipid particles for at least 3 months at 4° C. In one embodiment, the integrity of the nucleic acid (preferably RNA) in the nucleic acid-lipid particles does not decrease by more than 20% after storage of the nucleic acid-lipid particles for at least 6 months at −20° C.

Therefore, in one embodiment, there is provided a nucleic acid-lipid particle (preferably RNA-lipid particle) wherein the integrity of the nucleic acid (preferably RNA) in the nucleic acid-lipid particles does not decrease by more than 20% after storage of the nucleic acid-lipid particles for at least 3 months at 4° C. In one embodiment, there is provided a nucleic acid-lipid particle (preferably RNA-lipid particle) wherein the integrity of the nucleic acid (preferably RNA) in the nucleic acid-lipid particles does not decrease by more than 20% after storage of the nucleic acid-lipid particles for at least 6 months at −20° C.

In one embodiment, the nucleic acid-lipid particles are capable of inducing comparable or higher (e.g., 0.5 fold, 2 fold, 5 fold, 100 fold) antibody and/or T-cell responses after administration in vivo as compared to nucleic acid-lipid particles made using a standard process.

Therefore, in one embodiment, there is provided a nucleic acid-lipid particle (preferably RNA-lipid particle) wherein, the nucleic acid-lipid particle is capable of inducing comparable or higher (e.g., 0.5 fold, 2 fold, 5 fold, 100 fold) antibody and/or T-cell responses after administration in vivo as compared to nucleic acid-lipid particles made using a standard process.

### Method of Forming Nucleic Acid-Lipid Particle

In a further aspect, the present disclosure provides methods for producing the nucleic acid-lipid particles as disclosed herein. Generally, such methods comprise addition of the aqueous dispersion as described herein (typically containing pre-LNPs) to a composition containing a nucleic acid. In one embodiment, the composition containing the nucleic acid is a solution containing the nucleic acid. In one embodiment, the composition containing the nucleic acid is an aqueous solution containing the nucleic acid.

In one aspect, the method comprises:
i) preparing an aqueous dispersion, as defined herein, according to any of the methods defined herein; and
ii) mixing the aqueous dispersion with an aqueous solution comprising the nucleic acid, to produce the nucleic acid-lipid particle.

In one embodiment, the method of forming the RNA-lipid particle comprises:
i) preparing an aqueous dispersion as defined herein according to any of the methods defined herein; and
ii) mixing the aqueous dispersion with an aqueous solution comprising RNA, to produce the RNA-lipid particle.

In one embodiment, the method of forming the nucleic acid-lipid particle comprises:
i) mixing a lipid mixture comprising a cationic or cationically ionisable lipid dissolved in a water-soluble organic solvent with an aqueous phase, wherein the lipid solution and/or the aqueous phase is acidified, to produce an intermediate acidified aqueous lipid dispersion;
ii) performing on the intermediate dispersion a dialysis or filtration step at a pH of about 2.5 to about 5.5 (preferably about 2.5 to about 4.5), or at a pH of about 6.5 to about 8.5 (preferably about 7.5 or about 8.5), to remove the organic solvent and produce an aqueous dispersion,
wherein the aqueous dispersion is substantially free of acetate buffers, citrate buffers, organic solvents and RNA, and
wherein the aqueous dispersion comprises a cryoprotectant;
iii) mixing the aqueous dispersion with an aqueous solution comprising a nucleic acid, to produce the nucleic acid-lipid particle.

### Mixing Step

The mixing step of this aspect of the invention comprises mixing the aqueous dispersion, as defined herein (typically containing pre-LNPs) with an aqueous solution comprising a nucleic acid, as defined herein, to produce the nucleic acid-lipid particle.

In one embodiment, the aqueous dispersion is provided at neutral pH and either the aqueous dispersion or the aqueous solution is acidified.

In one embodiment, the aqueous dispersion is provided at acidic pH and neither the aqueous dispersion nor the aqueous solution is acidified.

In one embodiment, the volume ratio of the aqueous dispersion to the aqueous solution containing the nucleic acid is 1.5:1 to 1:1.5. In one embodiment, the volume ratio of the aqueous dispersion to the aqueous solution containing the nucleic acid is 1.2:1 to 1:1.2. In one embodiment, the volume ratio of the aqueous dispersion to the aqueous solution containing the nucleic acid is 1.1:1 to 1:1.1. In one embodiment, the volume ratio of the aqueous dispersion to the aqueous solution containing the nucleic acid is 1.05:1 to 1:1.05. In one embodiment, the volume ratio of the aqueous dispersion to the aqueous solution containing the nucleic acid is 1:1.

In a preferred embodiment, the acid is a water-soluble organic acid, as defined generally above. Examples of suitable organic acids include sulfonic acids, phosphoric acids, phosphonic acids, carboxylic acids, dicarboxylic acids, or hydroxy carboxylic acids (all as defined herein).

In one embodiment, the water-soluble organic acid is selected from the group consisting of acetic acid, malic acid, succinic acid, and citric acid, or combinations thereof. In one embodiment, the water-soluble organic acid may be selected from the group consisting of acetic acid and malic acid, or combinations thereof.

In one embodiment, the water-soluble organic acid is acetic acid. In one embodiment, the water-soluble organic acid is malic acid. In one embodiment, the water-soluble organic acid is succinic acid. In one embodiment, the water-soluble organic acid is citric acid.

In one embodiment, the concentration of the acid is in the range of about 0.1 to about 20 mM. In one embodiment, the concentration of the acid is in the range of about 0.2 to about 15 mM. In one embodiment, the concentration of the acid is in the range of about 0.5 to about 10 mM. In one embodiment, the concentration of the acid is in the range of about 1 to about 5 mM. In one embodiment, the concentration of the acid is in the range of about 2 to about 10 mM. In one embodiment, the concentration of the acid is in the range of about 0.5 to about 5 mM. In one embodiment, the concentration of the acid is in the range of about 3 to about 15 mM. In one embodiment, the concentration of the acid is in the range of about 5 to about 8 mM. In one embodiment, the concentration of the acid is in the range of about 8 to about 12 mM. It will be understood in this context that this concentration includes both the undissociated acid and its conjugate base.

In one embodiment, the acid is acetic acid and is present in a concentration in the range of about 0.2 to about 20 mM. In one embodiment, the acid is acetic acid and is present in a concentration in the range of about 0.5 to about 10 mM. In one embodiment, the acid is acetic acid and is present in a concentration in the range of about 0.5 to about 4 mM. In one embodiment, the acid is acetic acid and is present in a concentration in the range of about 1 to about 3.5 mM. In one embodiment, the acid is acetic acid and is present in a concentration in the range of about 2 to about 3 mM. In one embodiment, the acid is acetic acid and is present in a concentration of about 2.5 mM. In one embodiment, the acid is acetic acid and is present in a concentration in the range of about 1 to about 8 mM. In one embodiment, the acid is acetic acid and is present in a concentration in the range of about 1 to about 8 mM. In one embodiment, the acid is acetic acid and is present in a concentration in the range of about 2 to about 7 mM. In one embodiment, the acid is acetic acid and is present in a concentration in the range of about 4 to about 6 mM. In one embodiment, the acid is acetic acid and is present in a concentration in the range of about 4.5 to about 5.5 mM. In one embodiment, the acid is acetic acid and is present in a concentration of about 5 mM. In one embodiment, the acid is acetic acid and is present in a concentration in the range of about 5.5 to about 9 mM. In one embodiment, the acid is acetic acid and is present in a concentration in the range of about 6 to about 8.5 mM. In one embodiment, the acid is acetic acid and is present in a concentration in the range of about 7 to about 8 mM. In one embodiment, the acid is acetic acid and is present in a concentration of about 7.5 mM. In one embodiment, the acid is acetic acid and is present in a concentration in the range of about 6 to about 14 mM. In one embodiment, the acid is acetic acid and is present in a concentration in the range of about 7 to about 13 mM. In one embodiment, the acid is acetic acid and is present in a concentration in the range of about 8 to about 12 mM. In one embodiment, the acid is acetic acid and is present in a concentration in the range of about 9 to about 11 mM. In one embodiment, the acid is acetic acid and is present in a concentration of about 10 mM.

In one embodiment, the acid is malic acid and is present in a concentration in the range of about 0.1 to about 5 mM. In one embodiment, the acid is malic acid and is present in a concentration in the range of about 0.4 to about 4 mM. In one embodiment, the acid is malic acid and is present in a concentration in the range of about 0.8 to about 2 mM. In one embodiment, the acid is malic acid and is present in a concentration in the range of about 1 to about 1.5 mM. In one embodiment, the acid is malic acid and is present in a concentration of about 1.25 mM. In one embodiment, the acid is malic acid and is present in a concentration in the range of about 0.5 to about 4 mM. In one embodiment, the acid is malic acid and is present in a concentration in the range of about 1 to about 3.5 mM. In one embodiment, the acid is malic acid and is present in a concentration in the range of about 2 to about 3 mM. In one embodiment, the acid is malic acid and is present in a concentration of about 2.5 mM.

In one embodiment, the acid is citric acid and the concentration of the acid is greater than 0.3 mM. In one embodiment, the acid is citric acid and is present in a concentration in the range of about 0.2 to about 15 mM. In one embodiment, the acid is citric acid and is present in a concentration in the range of about 0.5 to about 10 mM. In one embodiment, the acid is citric acid and is present in a concentration in the range of about 1 to about 8 mM. In one embodiment, the acid is citric acid and is present in a concentration in the range of about 2 to about 7 mM. In one embodiment, the acid is citric acid and is present in a concentration in the range of about 4 to about 6 mM. In one embodiment, the acid is citric acid and is present in a concentration in the range of about 4.5 to about 5.5 mM. In one embodiment, the acid is citric acid and is present in a concentration of about 5 mM.

In one embodiment, the acid is succinic acid and is present in a concentration in the range of about 0.2 to about 10 mM. In one embodiment, the acid is succinic acid and is present in a concentration in the range of about 0.4 to about 5 mM. In one embodiment, the acid is succinic acid and is present in a concentration in the range of about 1 to about 3.5 mM. In one embodiment, the acid is succinic acid and is present in a concentration in the range of about 2 to about 3 mM. In one embodiment, the acid is succinic acid and is present in a concentration of about 2.5 mM.

In one preferred embodiment of any of the above methods of forming the nucleic acid-lipid particle, the aqueous dispersion comprises a cryoprotectant, as defined and exemplified herein. In one embodiment, the cryoprotectant is a carbohydrate. In one embodiment, the cryoprotectant is a monosaccharide or disaccharide. In one embodiment, the cryoprotectant is selected from the group consisting of sucrose, trehalose, lactose and glucose, or a mixture of any thereof. In one embodiment, the cryoprotectant is selected from the group consisting of sucrose, trehalose and glucose, or a mixture of any thereof. Preferably, the cryoprotectant is sucrose or trehalose.

When the aqueous dispersion comprises a cryoprotectant which is a carbohydrate, typically, this is present in a concentration of about 1% to about 30% (w/v).

In one embodiment, the cryoprotectant is sucrose or trehalose, and is present in a concentration of about 1% to about 30% (w/v), optionally about 3% to about 25% (w/v), preferably about 5% to about 20% (w/v). In one embodiment, the cryoprotectant is sucrose or trehalose, and is present in a concentration of about 8% to about 12% (w/v), such as about 10% (w/v). In one embodiment, the cryoprotectant is glucose and is present in a concentration of about 1% to about 15% (w/v), optionally about 3% to about 12% (w/v), preferably about 5% to about 10% (w/v).

In one embodiment, the cryoprotectant is sucrose or trehalose, and is present in a concentration of about 1% to about 30% (w/v), optionally about 10% to about 25% (w/v), preferably about 15% to about 25% (w/v). In one embodiment, the cryoprotectant is sucrose or trehalose, and is present in a concentration of about 18% to about 22% (w/v), such as about 20% (w/v). In one embodiment, the cryoprotectant is glucose and is present in a concentration of about 1% to about 15% (w/v), optionally about 5% to about 15% (w/v), preferably about 8% to about 12% (w/v). In such embodiments, preferably the nucleic acid-lipid particles are not subjected to a further dilution and/or addition of cryoprotectant step. For example, the nucleic acid-lipid particles may not require any further processing steps.

In one embodiment, the methods of the invention for forming the nucleic acid-lipid particle are performed at from about 0° C. to about 25° C., optionally from about 4° C. to about 25° C., preferably from about 15° C. to about 25° C. In one embodiment, the methods of the invention for producing the aqueous dispersion are performed at about room temperature (e.g., 18-25° C.).

In one embodiment, the aqueous solution containing the nucleic acid also contains one or more buffering agents. In one embodiment, the buffering agent is 4-(2-hydroxy-ethyl)-1-piperazineethanesulfonic acid (HEPES), optionally in combination with ethylenediaminetetraacetic acid (EDTA) or an acceptable salt thereof. The aqueous solution containing the nucleic acid may have a pH of from about 6.5 to about 8.5, optionally from about 6.8 to about 7.5. The aqueous solution containing the nucleic acid may have a pH of about 7.0.

### Optional Further Processing Steps

In one embodiment, the method comprises further subjecting the nucleic acid-lipid particle to one or more further processing steps.

In one embodiment, the method further comprises adding peptide-conjugated lipid (as further described herein) to the nucleic acid-lipid particles. In some instances, the peptide-conjugated lipid may displace (i.e., replace) a corresponding portion of the steroid (e.g., cholesterol) in the nucleic acid-lipid particles. The composition of the nucleic acid-lipid particles before addition of peptide-conjugated lipid may comprise a cationic or cationically ionizable lipid as described herein, a neutral or zwitterionic phospholipid as descried herein, a steroid as described herein and optionally a grafted lipid as described herein, in a molar ratio of 20-70 mol %:5-15 mol %:20-60 mol % and optionally 0.5-10 mol %, respectively; optionally 40-60 mol %:8-12 mol %:30-50 mol % and optionally 1.0-5 mol %, respectively. Following addition of the peptide-conjugated lipid, the peptide-conjugated lipid may comprise 0.05-1.0 mol %, optionally 0.1 to 0.5 mol %, preferably 0.1-0.3 mol % of the lipid in the nucleic acid-lipid particles, with a corresponding reduction in the mol % of the steroid.

In one embodiment, the method comprises further subjecting the nucleic acid-lipid particle to one or more purification steps. In one embodiment, the purification step comprises a dialysis or filtration step. In one embodiment, the dialysis or filtration step comprises tangential flow filtration. In one embodiment, the method does not comprise subjecting the nucleic acid-lipid particle to a filtration or dialysis step. In one embodiment, the method does not comprise subjecting the nucleic acid-lipid particle to a tangential flow filtration step.

In one embodiment, the method comprises further subjecting the nucleic acid-lipid particle to one or more dilution steps. In one embodiment, the one or more dilution steps comprise addition of cryoprotectant. In one preferred embodiment, the method does not comprise subjecting the nucleic acid-lipid particle to any of (i) a dialysis or filtration step (e.g., a TFF step), (ii) a dilution step, and (iii) a dilution step comprising addition of cryoprotectant. In one embodiment, the cryoprotectant is selected from the group consisting of sucrose, glycerol, trehalose, lactose, glucose and mannitol. In one embodiment, the cryoprotectant is sucrose.

In one embodiment, the purification step is carried out using aqueous phase essentially free of buffering agents.

In one embodiment, the method further comprises the step of sterile filtration of the nucleic acid-lipid particle. Typically, the sterile filtration uses a 0.22 μm filter. In one embodiment, the filter is a polyethersulfone (PES) filter.

In one embodiment, the method further comprises the step of drying of the nucleic acid-lipid particle. In one embodiment, the drying is freeze drying. In one embodiment, the drying is spray drying.

In one embodiment, the one or more purification steps for the nucleic acid-lipid particle do not comprise a tangential flow filtration step.

In one embodiment, the nucleic acid-lipid particles are not subjected to any further purification steps.

In one embodiment, the method further comprises a step of diluting the lipid particles with a storage matrix. In one embodiment, the storage matrix comprises one or more buffering agents. In one embodiment, the buffering agent or mixture thereof has a pH of 4.5 to 8.5. In one embodiment, the buffering agent is selected from the group consisting of 4-(2-hydroxyethyl)-1-piperazineethanesulfonic acid (HEPES), tris-(hydroxymethyl)aminomethane (Tris), histidine, triethanolamine, or a mixture of any thereof. In one embodiment, the buffering agent is a mixture of HEPES and Tris. Preferred molar ratios of HEPES:Tris are between 100:1 to 1:100, preferably 10:1 to 1:10.

### Lipids and Amphiphiles

The compositions of the invention also contain a mixture of lipids. The terms “lipid” and “lipid-like material” are broadly defined herein as molecules which comprise one or more hydrophobic moieties or groups and also one or more hydrophilic moieties or groups.

Lipids are usually insoluble or poorly soluble in water, but soluble in many organic solvents. In an aqueous environment, the amphiphilic nature allows the molecules to self-assemble into organized structures and different phases.

Lipids may comprise a polar portion and an apolar (or non-polar) portion. The term “amphiphile” as used in this specification is broadly defined herein as a molecule comprising hydrophobic moieties and hydrophilic moieties and/or a polar and apolar portion. As both cationic and anionic lipids both contain such groups, they are therefore amphiphiles. In this specification the term “cationic lipid” is therefore synonymous with “cationic amphiphile” and the term “anionic lipid” is synonymous with “anionic amphiphile”.

Hydrophobicity can be conferred by the inclusion of apolar groups that include, but are not limited to, long-chain saturated and unsaturated hydrocarbyl groups (as defined and exemplified above), such as alkyl, alkenyl and/or alkynyl groups and such groups substituted by one or more aryl, heteroaryl, or cycloalkyl groups (as defined and exemplified above). The hydrophilic groups may comprise polar and/or charged groups and include at least one amine and optionally hydrophilic non-charged groups such as hydroxyl, carbohydrate, sulfhydryl, nitro or like groups and may further include anionic groups such as phosphate, phosphonate, carboxylic acid, sulfate, sulfonate (all as defined and exemplified above) and other like groups.

The term “hydrophobic” as used herein with respect to a compound, group or moiety means that said compound, group, or moiety is not attracted to water molecules and, when present in an aqueous solution, excludes water molecules. In some embodiments, the term “hydrophobic” refers to any compound, group or moiety which is substantially immiscible or insoluble in aqueous solution. In some embodiments, a hydrophobic compound, group or moiety is substantially nonpolar.

Examples of hydrophobic groups are hydrocarbyl groups (as defined and exemplified above), such as alkyl, alkenyl and/or alkynyl groups and such groups substituted by one or more aryl, heteroaryl, or cycloalkyl groups (as defined and exemplified above). The hydrophobic group can have functional groups (e.g., ether, thioether, ester, dioxolane, halide, amide, sulfonamide, carbamate, etc.) and atoms other than carbon and hydrogen as long as the group satisfies the condition of being substantially immiscible or insoluble in aqueous solution.

The hydrophobic moieties of a lipid may have between 24 and 60 carbon atoms and can be hydrocarbyls (as described and exemplified above, typically comprising alkyl, alkenyl or alkynyl groups as described and exemplified above). The 24 to 60 carbon atoms can be segmented into two or more hydrophobic moieties, with each such moiety typically having at least 6 carbon atoms. An example for segmented hydrophobic moieties wherein each segment is hydrocarbyl are lipids comprising the DACA moiety as described in WO2011/003834 wherein each of the acyl or alkyl groups comprise between 12 and 20 carbon atoms. Another example are lipids wherein the hydrophobic moiety comprises a steroid moiety, such as a cholesteryl moiety.

The hydrophobic moieties of a lipid preferably have between 24 and 60 carbon atoms and can also be heterohydrocarbyls wherein the heteroatoms are selected from N, O or S forming one, two, three or four non-charged groups of ether, thioether, ester, amide, carbamate, sulfonamide and the like. The 24 to 60 carbon atoms can be segmented into two or more hydrophobic moieties, provided that each such moiety has at least 6 carbon atoms. An example for segmented hydrophobic moieties wherein each segment is hydrocarbyl are lipids comprising the diacylglycerol or dialkylglycerol moiety wherein each of the acyl or alkyl comprise between 12 and 20 carbon atoms. An example for hydrophobic moieties wherein each segment is heterohydrocarbyl are the ester-branched moieties in lipids such as SM-102 or ALC-315, as defined and exemplified below.

### Cationic and Cationically Ionizable Lipids

The aqueous dispersions and lipid particles of the present invention also contain a cationic lipid or cationically ionizable lipid, or a mixture of any thereof. In one embodiment the aqueous dispersions and lipid particles of the present comprise a cationically ionizable lipid, and preferably do not comprise a cationic lipid.

As used herein, the term “cationic lipid” means a lipid or lipid-like material, as defined herein, having a constitutive positive charge. In this context a “constitutive charge” means that the cationic lipid carries the positive charge at all physiological pH. The cationic lipids carrying constitutive charged cationic moieties are typically quaternary ammonium salts (as defined above) or salts of organic bases, such as nitrogen-containing bases. Typically, such organic bases are strong bases (i.e. bases which are completely protonated when dissolved in a solvent, such as but not limited to an aqueous solvent, such that the concentration of the unprotonated species is too low to be measured).

In one embodiment, the cationic lipid is a monovalent cationic lipid.

In one embodiment, the cationic lipid contains a charged polar moiety selected from the group consisting of guanidinium, ammonium, imidazolium, pyridinium, amidinium, and piperazinium.

Examples of cationic lipids include, but are not limited to 1,2-dialkyloxy-3-dimethylammonium propanes and 1,2-dialkenyloxy-3-dimethylammonium propanes (each alkyl or alkenyl portion being as defined and exemplified above and preferably having 12 to 20 carbon atoms), such as 1,2-di-O-octadecenyl-3-trimethylammonium propane (DOTMA), 1,2-diacyloxy-3-dimethylammonium propanes (the alkyl or alkenyl part of each acyl portion being as defined and exemplified above and preferably having 12 to 20 carbon atoms), such as 1,2-dioleoyl-3-trimethylammonium propane (DOTAP) or 1,2-dioleoyl-3-dimethylammonium-propane (DODAP); dimethyldioctadecylammonium (DDAB); dioctadecyldimethyl ammonium chloride (DODAC), 2,3-di(tetradecoxy)propyl-(2-hydroxyethyl)-dimethylazanium (DMRIE); 1,2-dimyristoyl-sn-glycero-3-ethylphosphocholine (DMEPC), 1,2-dimyristoyl-3 -trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA).

The structures of DODMA and DODAP are shown below.

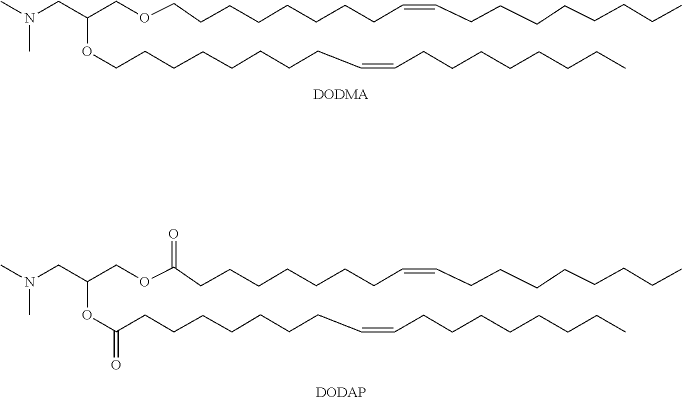

The structures of DOTMA, DOTAP and analogues thereof are shown below.

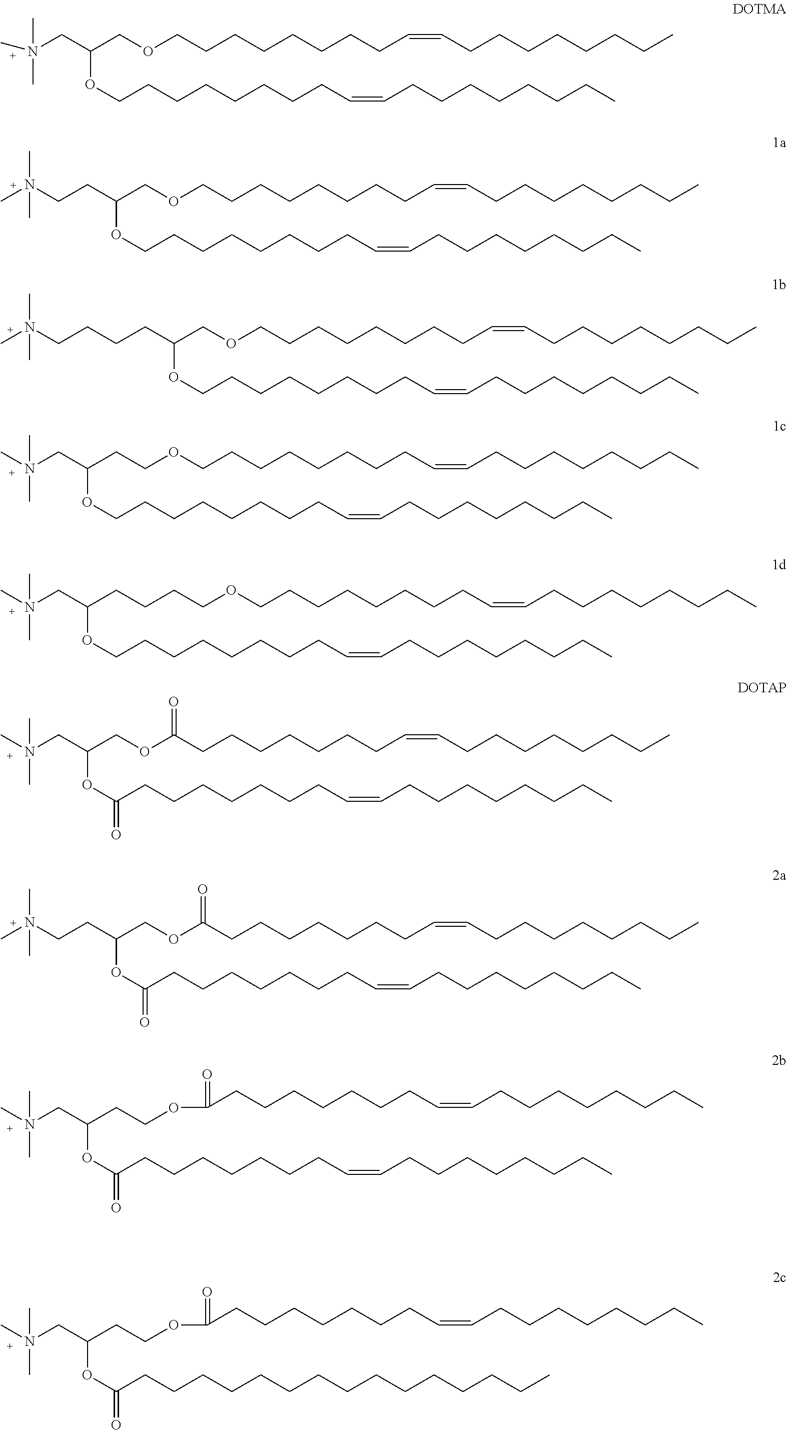

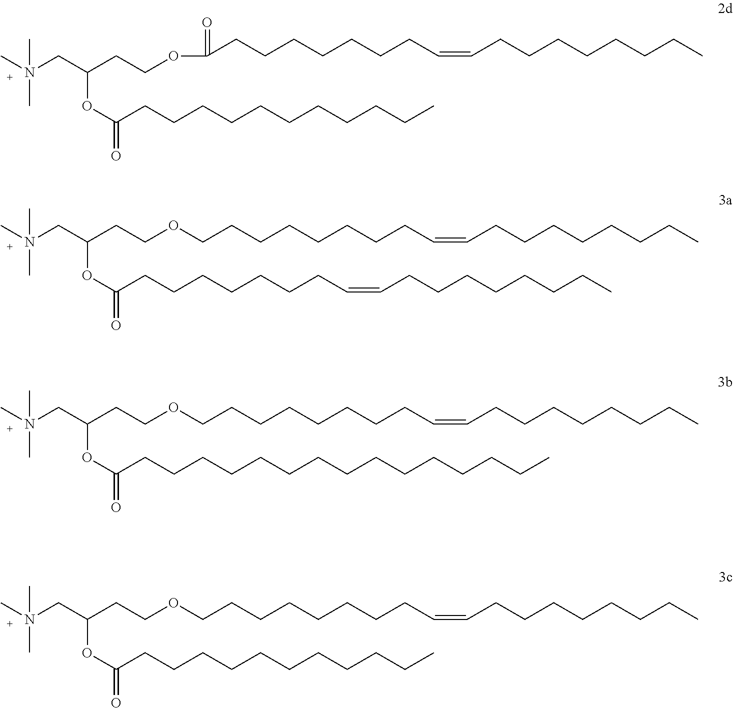

The structures of DOTAP and further suitable homologues are shown below.

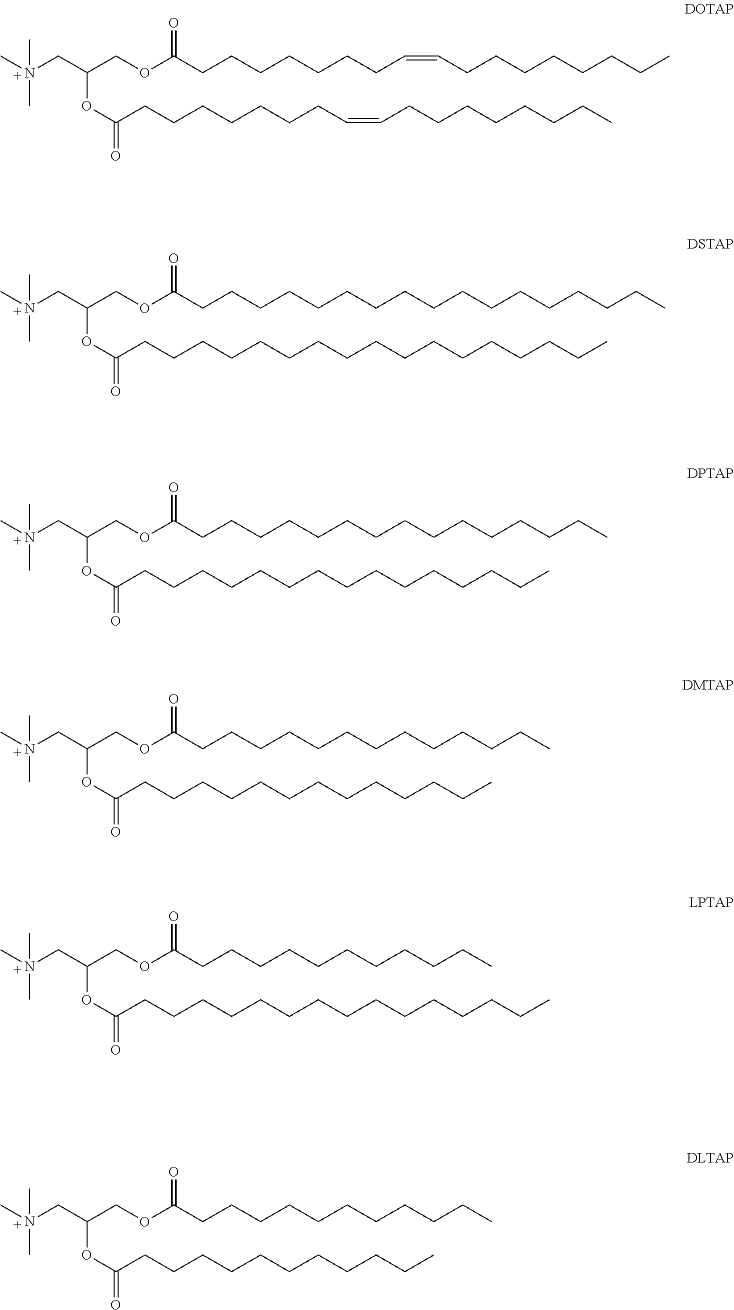

The structures of DOTMA, DORIE and further suitable homologues are shown below.

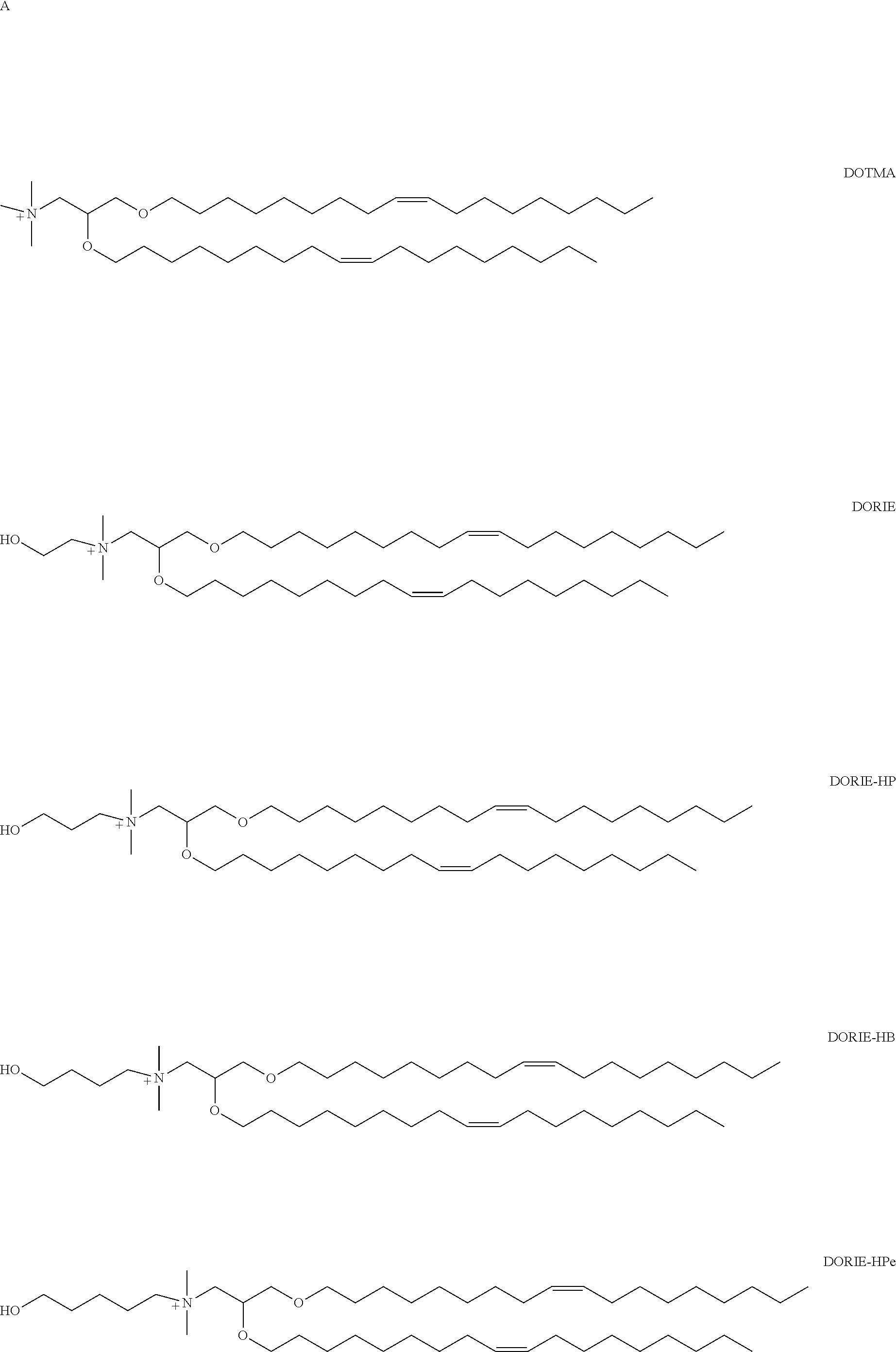

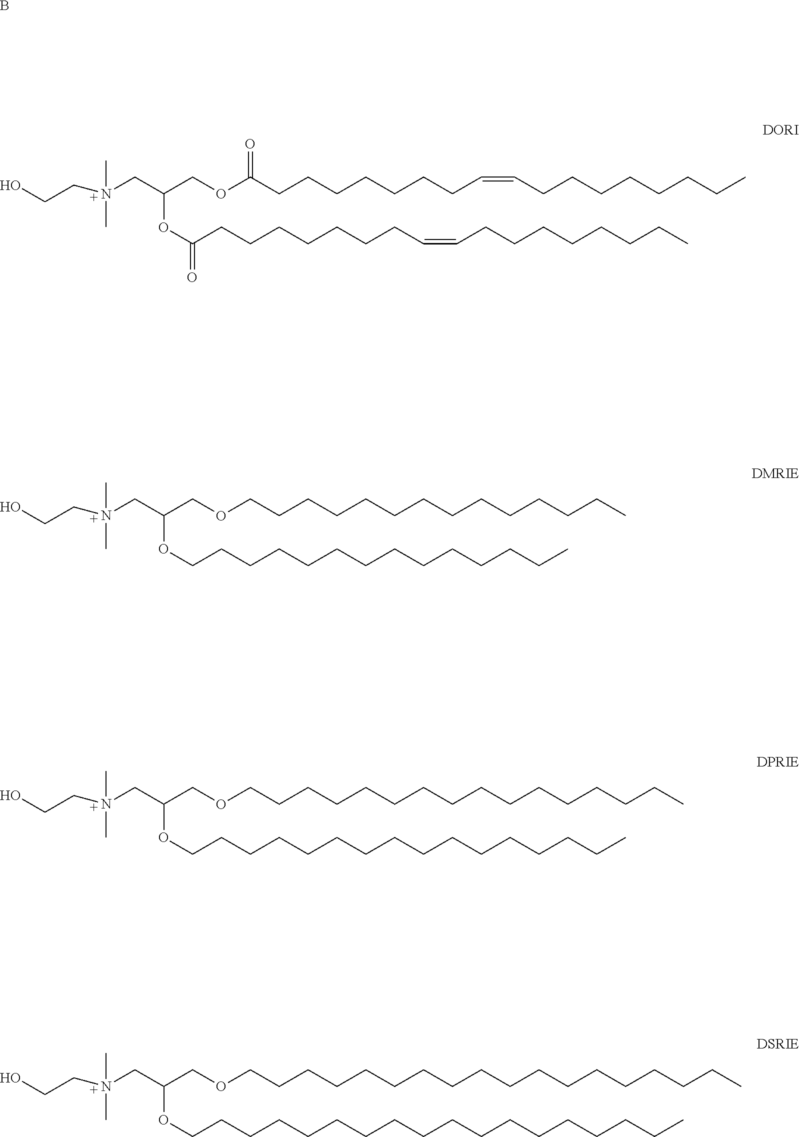

Further suitable cationic lipids are described in Sun and Lu, Pharmaceutical Research, 2023, https://doi.org/10.1007/s11095-022-03460-2.

In one embodiment, the lipid is a cationically ionizable lipid. As used herein, a “cationically ionizable lipid” refers to a lipid or lipid-like material which, depending on whether it is protonated or deprotonated, has a net positive charge or is neutral, i.e., a lipid which is not permanently cationic. Thus, depending on the pH of the composition in which the cationically ionizable lipid is solved, the cationically ionizable lipid is either positively charged or neutral.

In some embodiments, the cationically ionizable lipid comprises a head group which includes at least one nitrogen atom (N) which is capable of being protonated, preferably under physiological or slightly acidic conditions.

In one embodiment, the cationically ionizable lipid is a compound represented by formula (TL-I):

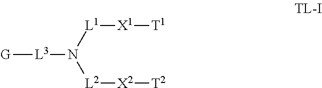

or a pharmaceutically acceptable salt thereof, wherein:
L¹ and L² are each independently an optionally substituted C₁-C₃₀ aliphatic group; L³ is a bond, optionally substituted C₁-C₁₀ aliphatic group, or optionally substituted 2- to 10-membered heteroaliphatic group;
X¹ and X² are each independently selected from a bond, —OC(O)—, —C(O)O—, —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—, wherein one or both of X¹ or X² is selected from —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—;
each R¹ is, independently, at each instance, optionally substituted C₁-C₂₀ aliphatic or H;
T¹ and T² are each independently an optionally substituted C₃-C₃₀ aliphatic;
G is —N(R²)C(S)N(R²)₂, —N⁺(R³)₃, —OH, —N(R²)₂, —N(R⁵)C(O)R³, —N(R⁵)S(O)₂R³, —N(R⁵)C(O)N(R³)₂, —CH(N—R²), or —R⁴;
each R² is, independently, at each instance, selected from the group consisting of H, optionally substituted C₁-C₆ aliphatic or OR³; or two instances of R² come together with the atoms to which they are attached to form an optionally substituted 4- to 12-membered heterocycle ring or an optionally substituted 4- to 12-membered heteroaryl ring;
each R³ is, independently, at each instance, selected from the group consisting of H and optionally substituted C₁-C₁₀ aliphatic; and
R⁴ is optionally substituted 4- to 12-membered heterocycle, optionally substituted 4- to 12 membered heteroaryl, C₆-C₁₂ aryl substituted with one or more of —(CH₂)₀₋₆—OH or —(CH₂)₀₋₆—N(R⁵)₂, or C₃-C₁₂ cycloaliphatic substituted with one or more of oxo, —(CH₂)₀₋₆—OH, or —(CH₂)₀₋₆—N(R⁵)₂;
each R⁵ is independently selected from H and optionally substituted C₁-C₆ aliphatic.

In some embodiments of formula (TL-I), L¹ and L² are each independently —(CH₂)₆₋₁₀—.

In some embodiments of formula (TL-I), X¹ and X² are each independently selected from a —S(O)₂N(R¹)—, —N(R¹)S(O)₂, —S(O)—, —S(O)₂—, —S(O)₂C(R¹)₂—, —OC(S)C(R¹)₂—, —C(R¹)₂C(S)O—, and —S—.

In some embodiments of formula (TL-I), X¹ and X² are each —S(O)₂N(R¹)—, where each R¹ is independently R¹ is C₁-C₁₀ aliphatic.

In some embodiments of formula (TL-I), T¹ and T² are each independently selected from optionally substituted C₃-C₂₀ alkyl.

In some embodiments of formula (TL-I), T¹ and T² are each independently selected from:

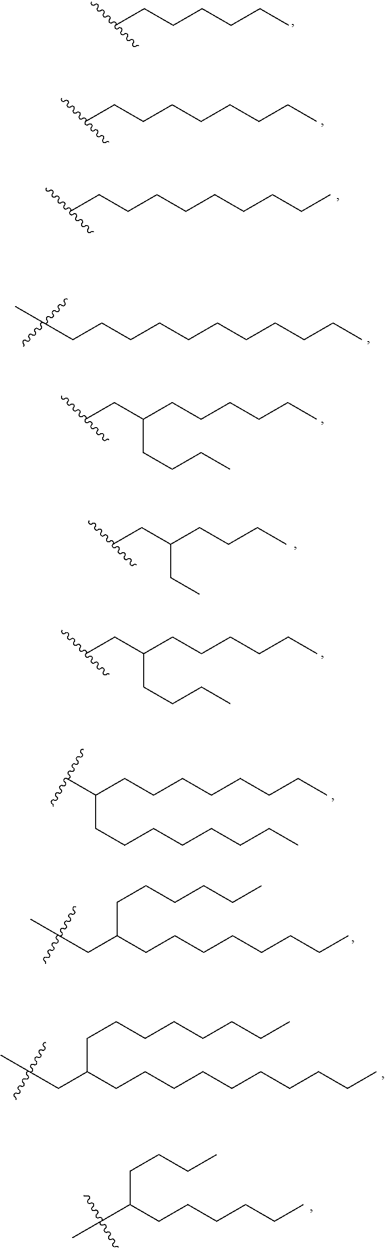

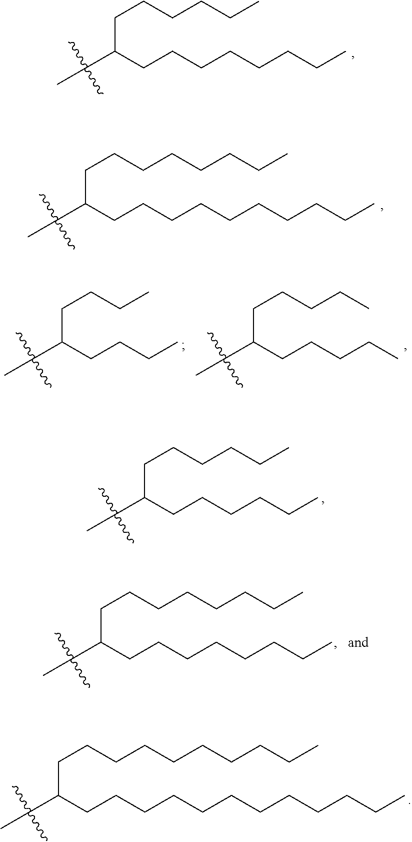

In some embodiments of formula (TL-I), G is —N(R²)C(S)N(R²)₂ or —N(R⁵)S(O)₂R³.

In some embodiments of formula (TL-I), G is —N(H)C(S)N(R²)₂, where each R² is selected from optionally substituted C₁-C₆ aliphatic and OH.

In some embodiments of formula (TL-I), G is —OH.

In some embodiments of formula (TL-I), G is selected from:

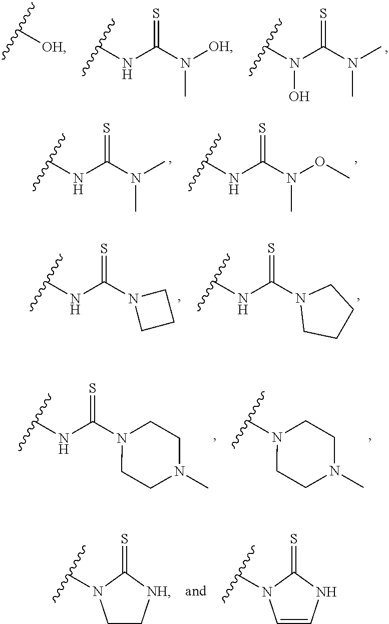

In some embodiments of formula (TL-I), -L³-G is selected from:

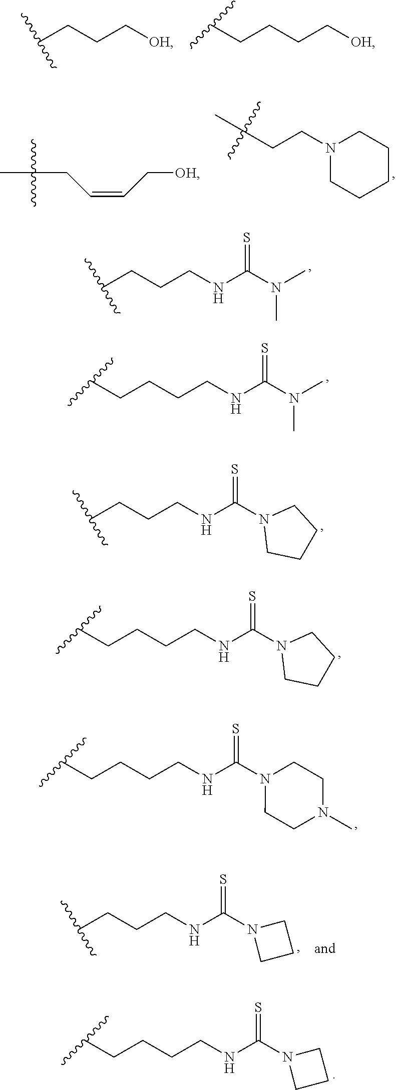

In some embodiments of formula (TL-I), the compound is represented by Formula (TL-IIa):

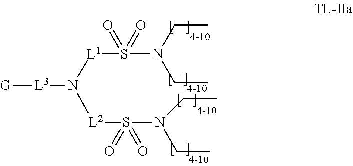

or a pharmaceutically acceptable salt thereof.

In some embodiments of formula (TL-I), the compound is represented by Formula (TL-IIc):

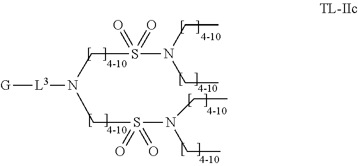

or a pharmaceutically acceptable salt thereof.

In some embodiments of formula (TL-I), the compound is represented by Formula (TL-IIIb):

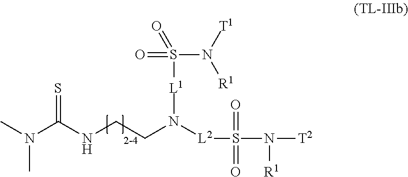

or a pharmaceutically acceptable salt thereof.

In some embodiments of formula (TL-I), the compound is represented by Formula (TL-IIIe):

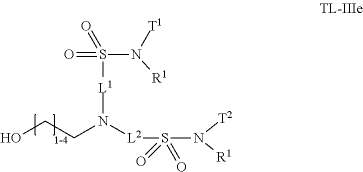

or a pharmaceutically acceptable salt thereof.

In some embodiments of formula (TL-I), the compound is 7,7′-((4-hydroxybutyl)azanediyl)bis(N-hexyl-N-octylheptane-1-sulfonamide)

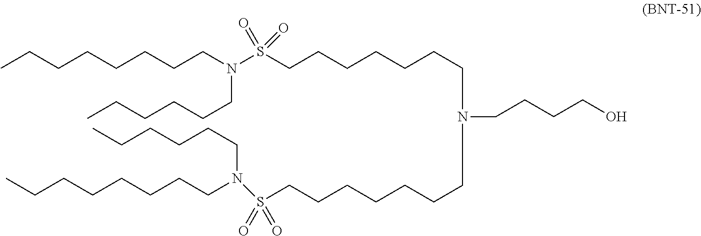 [SMILES: CCCCCCCCN(CCCCCC)S(=O)(=O)CCCCCCCN(CCCCO)CCCCCCCS(=O)(=O)N(CCCCCC)CCCCCCCC]

or a pharmaceutically acceptable salt thereof.

In some embodiments of formula (TL-I), the compound is 7,7′-((4-(3,3-dimethylthioureido)butyl)azanediyl)bis(N-hexyl-N-octylheptane-1-sulfonamide)

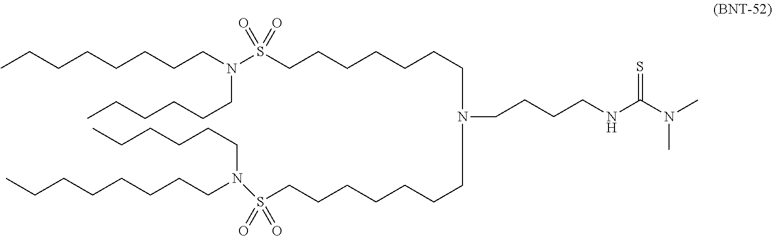 [SMILES: CCCCCCCCN(CCCCCC)S(=O)(=O)CCCCCCCN(CCCCCCCS(=O)(=O)N(CCCCCC)CCCCCCCC)CCCCNC(=S)N(C)C]

or a pharmaceutically acceptable salt thereof.

In some embodiments of formula (TL-I), the compound is

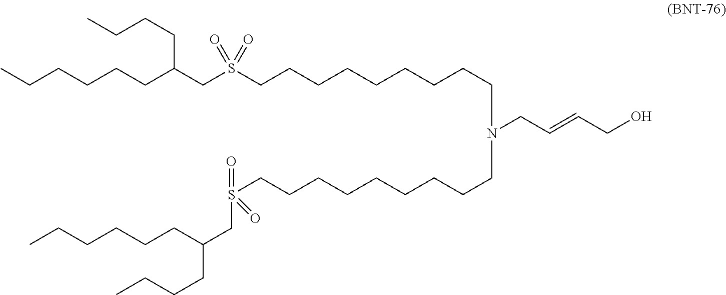 [SMILES: CCCCCCC(CCCC)CS(=O)(=O)CCCCCCCCCN(C/C=C/CO)CCCCCCCCCS(=O)(=O)CC(CCCC)CCCCCC]

or a pharmaceutically acceptable salt thereof.

Thiolipid compounds of formula (TL-I) can be prepared according to PCT/EP2023/071270, the contents of which are incorporated herein by reference.

In one embodiment, the cationic or cationically ionizable lipid is selected from the group consisting of:
[(4-hydroxybutyl)azanediyl]di(hexane-6,1-diyl) bis(2-hexyldecanoate) (ALC-315);
1,2-dioleoyloxy-3-dimethylaminopropane (DODMA); 2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
O—[N-{(9Z,12Z)-octadeca-9,12-dien-1-yl)}-N-{7-pentadecylcarbonyloxyoctyl}-amino]4-(dimethylamino)butanoate (HY501);
2-(di-((9Z,12Z)-octadeca-9,12-dien-1-yl)amino)ethyl 4-(dimethylamino)butanoate (EA-2);
4-((di-((9Z,12Z)-octadeca-9,12-dien-1-yl)amino)oxy)-N,N-dimethyl-4-oxobutan-4-amine (HYAM-2);
((2-(4-(dimethylamino)butanoyl)oxy)ethyl)azanediylbis(octane 8,1-diyl) bis(2-hexyldecanoate) (EA-405);
(2-(4-(dimethylamino)butanoyl)oxy)azanediylbis(octane 8,1-diyl) bis(2-hexyldecanoate) (HY-405);
palmitoyl-oleoyl-nor-arginine (PONA);
guanidino-di[(heptadecyl)methyl]carboxylic acid (GUADACA);
4-methylpyridinium-di(heptadecyl)methylcarboxylic acid (MPDACA);
1,2-dioleoyl-3 trimethylammonium propane (DOTAP);
1,2-dioleoyl-3-dimethylammomium propane (DODAP);
1,2-di-O-octadecenyl-3-trimethylammonium propane (DOTMA);
   or a mixture of any thereof.

In one embodiment, the cationically ionizable lipid is selected from the group consisting of:
7,7′-((4-hydroxybutyl)azanediyl)bis(N-hexyl-N-octylheptane-1-sulfonamide)

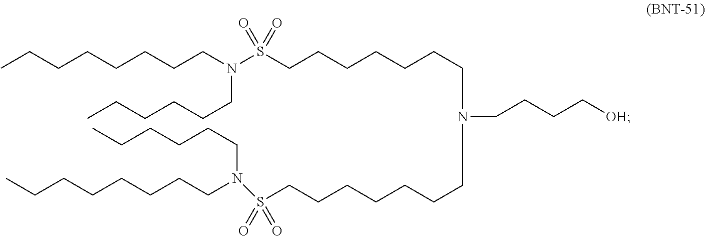 [SMILES: CCCCCCCCN(CCCCCC)S(=O)(=O)CCCCCCCN(CCCCO)CCCCCCCS(=O)(=O)N(CCCCCC)CCCCCCCC]

7,7′-((4-(3,3-dimethylthioureido)butyl)azanediyl)bis(N-hexyl-N-octylheptane-1-sulfonamide)

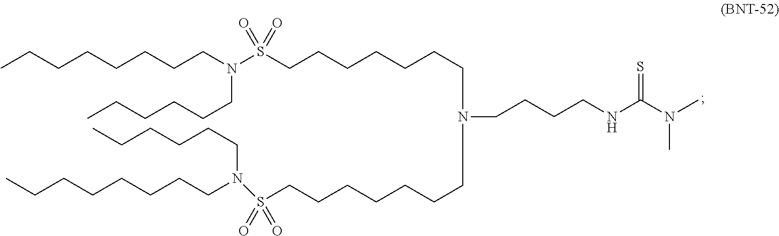 [SMILES: CCCCCCCCN(CCCCCC)S(=O)(=O)CCCCCCCN(CCCCCCCS(=O)(=O)N(CCCCCC)CCCCCCCC)CCCCNC(=S)N(C)C]

the compound having the structure

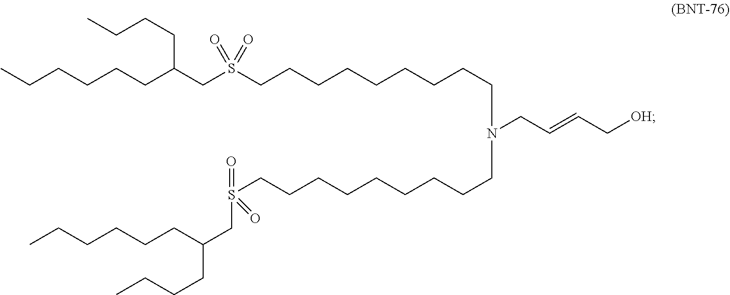 [SMILES: CCCCCCC(CCCC)CS(=O)(=O)CCCCCCCCCN(C/C=C/CO)CCCCCCCCCS(=O)(=O)CC(CCCC)CCCCCC]

[(4-hydroxybutyl)azanediyl]di(hexane-6,1-diyl) bis(2-hexyldecanoate) (ALC-315);
1,2-dioleoyloxy-3-dimethylaminopropane (DODMA);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
O—[N-{(9Z,12Z)-octadeca-9,12-dien-1-yl)}-N-{7-pentadecylcarbonyloxyoctyl}-amino]4-(dimethylamino)butanoate (HY501);
2-(di-((9Z,12Z)-octadeca-9,12-dien-1-yl)amino)ethyl 4-(dimethylamino)butanoate (EA-2);
4-((di-((9Z,12Z)-octadeca-9,12-dien-1-yl)amino)oxy)-N,N-dimethyl-4-oxobutan-4-amine (HYAM-2);
((2-(4-(dimethylamino)butanoyl)oxy)ethyl)azanediylbis(octane 8,1-diyl) bis(2-hexyldecanoate) (EA-405);
(2-(4-(dimethylamino)butanoyl)oxy)azanediylbis(octane 8,1-diyl) bis(2-hexyldecanoate) (HY-405);
di(heptadecan-9-yl) 3,3′-((2-(4-methylpiperazin-1-yl)ethyl)azanediyl)dipropionate

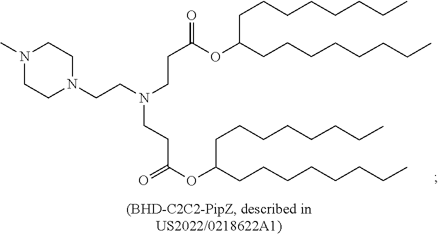 [SMILES: CCCCCCCCC(CCCCCCCC)OC(=O)CCN(CCC(=O)OC(CCCCCCCC)CCCCCCCC)CCN1CCN(C)CC1]

bis(2-octyldodecyl) 3,3′-((2-(1-methylpyrrolidin-2-yl)ethyl)azanediyl)dipropionate

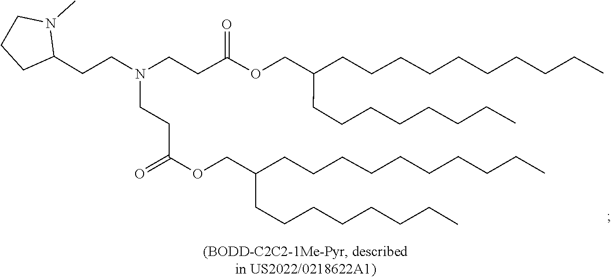 [SMILES: CCCCCCCCCCC(CCCCCCCC)COC(=O)CCN(CCC(=O)OCC(CCCCCCCC)CCCCCCCCCC)CCC1CCCN1C]

or a mixture of any thereof.

In one embodiment, the cationically ionisable lipid is selected from the group consisting of:
7,7′-((4-hydroxybutyl)azanediyl)-bis(N-hexyl-N-octylheptane-1-sulfonamide) (BNT-51);
7,7′-((4-(3,3-dimethylthioureido)butyl)azanediyl)bis(N-hexyl-N-octylheptane-1-sulfonamide) (BNT-52);
   the compound having the structure

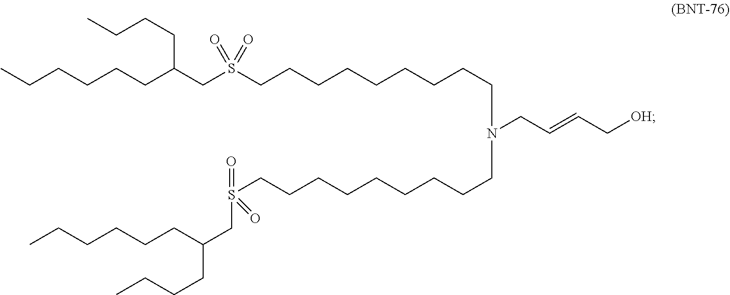 [SMILES: CCCCCCC(CCCC)CS(=O)(=O)CCCCCCCCCN(C/C=C/CO)CCCCCCCCCS(=O)(=O)CC(CCCC)CCCCCC]

[(4-hydroxybutyl)azanediyl]di(hexane-6,1-diyl) bis(2-hexyldecanoate) (ALC-0315);
1,2-dioleoyloxy-3-dimethylaminopropane (DODMA);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
0-[N-{(9Z,12Z)-octadeca-9,12-dien-1-yl)}-N-{7-pentadecylcarbonyloxyoctyl}-amino]4-(dimethylamino)butanoate (HY501);
((2-(4-(dimethylamino)butanoyl)oxy)ethyl)azanediylbis(octane 8,1-diyl) bis(2-hexyldecanoate) (EA-405);
(2-(4-(dimethylamino)butanoyl)oxy)azanediylbis(octane 8,1-diyl) bis(2-hexyldecanoate) (HY-405);
di(heptadecan-9-yl) 3,3′-((2-(4-methylpiperazin-1-yl)ethyl)azanediyl)dipropionate (BHD-C2C2-PipZ);
bis(2-octyldodecyl) 3,3′-((2-(1-methylpyrrolidin-2-yl)ethyl)azanediyl)dipropionate (BODD-C2C2-1Me-Pyr);
bis(2-octyldodecyl) 3,3′-((2-(pyrrolidin-1-yl)ethyl)azanediyl)dipropionate (BODD-C₂C₂-Pyr);
bis(2-octyldodecyl) 3,3′-((2-(1-methylpyrrolidin-2-yl)ethyl)azanediyl)dipropionate (BODD-C2C2-1MePyr);
bis(2-octyldodecyl) 3,3′-(((1-methylpiperidin-3-yl)methyl)azanediyl)dipropionate (BODD-C2C2-1Me-3PipD);
bis(2-octyldodecyl) 3,3′-((2-(dimethylamino)ethyl)azanediyl)dipropionate (BODD-C₂C₂-DMA);
bis(2-octyldodecyl) 3,3′-((4-(4-methylpiperazin-1-yl)butyl)azanediyl)dipropionate (BODD-C2C4-PipZ);
bis(2-octyldodecyl) 3,3′-((4-(pyrrolidin-1-yl)butyl)azanediyl)dipropionate (BODD-C₂C₄-Pyr);
bis(2-hexyldecyl) 3,3′-((4-(4-methylpiperazin-1-yl)butyl)azanediyl)dipropionate (BHD-C2C4-PipZ);
di(nonadecan-9-yl) 3,3′-((4-(4-methylpiperazin-1-yl)butyl)azanediyl)dipropionate (DND-C₂-C₄-PipZ);
   or a mixture of any thereof.

In one embodiment, the cationic lipid is palmitoyl-oleoyl-nor-arginine (PONA). In one embodiment, the cationic lipid is 4-methylpyridinium-di(heptadecyl)-methylcarboxylic acid (MPDACA). In one embodiment, the cationic lipid is 1,2-dioleoyloxy-3-trimethylammonium propane (DOTAP). In one embodiment, the cationic lipid is 1,2-dioleoyl-3-dimethylammonium-propane (DODAP).

In one embodiment, the cationically ionizable lipid is [(4-hydroxybutyl)azanediyl]-di(hexane-6,1-diyl) bis(2-hexyldecanoate) (ALC-315). In one embodiment, the cationically ionizable lipid is 1,2-dioleoyloxy-3-dimethylaminopropane (DODMA). In one embodiment, the cationically ionizable lipid is 2,2-dilinoleyl-4-dimethylaminoethyl-[1,3]-dioxolane (DLin-KC2-DMA). In one embodiment, the cationically ionizable lipid is heptatriaconta-6,9,28,31-tetraen-19-yl-4-(dimethylamino)butanoate (D-Lin-MC3-DMA). In one embodiment, the cationically ionizable lipid is 1,2-dilinoleyloxy-N,N-dimethylaminopropane (DLin-DMA)/In one embodiment, the cationically ionizable lipid is di((Z)-non-2-en-1-yl)-9-((4-(dimethylaminobutanoyl)oxy)heptadecanedioate (L319). In one embodiment, the cationically ionizable lipid is bis-(2-butyloctyl) 10-(N-(3-(dimethylamino)propyl)-nonanamido)-nonadecanedioate (A9). In one embodiment, the cationically ionizable lipid is (heptadecan-9-yl 8-{(2-hydroxyethyl)[6-oxo-6-(undecyloxy)octyl]amino}-octanoate) (L5). In one embodiment, the cationically ionizable lipid is heptadecan-9-yl 8-{(2-hydroxyethyl)[6-oxo-6-(undecyloxy)hexyl]amino}-octanoate) (SM-102). In one embodiment, the cationically ionizable lipid is O—[N-{(9Z,12Z)-octadeca-9,12-dien-1-yl)}-N-{7-pentadecylcarbonyloxyoctyl}-amino]4-(dimethylamino)butanoate (HY501). In one embodiment, the cationically ionizable lipid is 2-(di-((9Z,12Z)-octadeca-9,12-dien-1-yl)amino)ethyl 4-(dimethylamino)butanoate (EA-2). In one embodiment, the cationically ionizable lipid is 7,7′-((4-hydroxybutyl)azanediyl)-bis(N-hexyl-N-octylheptane-1-sulfonamide) (BNT-51). In one embodiment, the cationically ionizable lipid is 7,7′-((4-(3,3-dimethylthioureido)butyl)azanediyl)bis(N-hexyl-N-octylheptane-1-sulfonamide) (BNT-52). In one embodiment, the cationically ionizable lipid is the compound having the structure

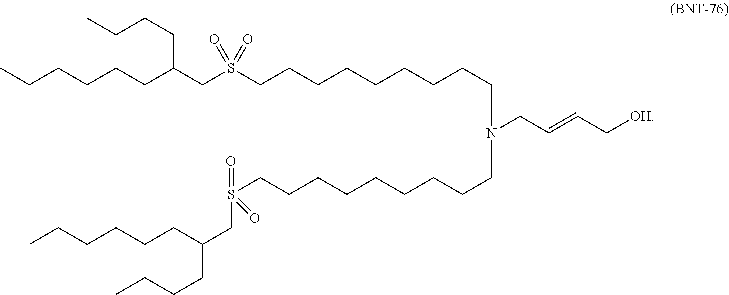 [SMILES: CCCCCCC(CCCC)CS(=O)(=O)CCCCCCCCCN(C/C=C/CO)CCCCCCCCCS(=O)(=O)CC(CCCC)CCCCCC]

In one embodiment, the cationically ionizable lipid is BHD-C2C2-PipZ. In one embodiment, the cationically ionizable lipid is BODD-C2C2-1Me-Pyr.

In some embodiments, the cationically ionizable lipid is selected from those described generally and specifically in WO 2018/087753.

In some embodiments, the cationically ionizable lipid is selected from the group consisting of:

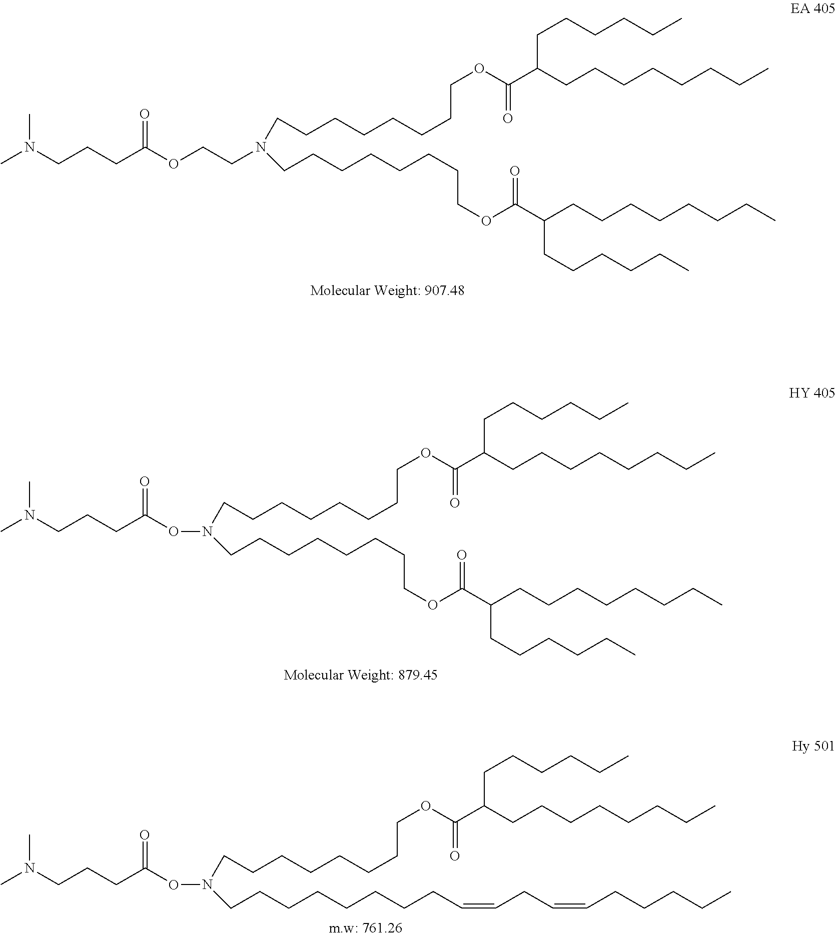

In one embodiment, the cationically ionizable lipid is 4-((di-((9Z,12Z)-octadeca-9,12-dien-1-yl)amino)oxy)-N,N-dimethyl-4-oxobutan-4-amine (HYAM-2). In one embodiment, the cationically ionizable lipid is ((2-(4-(dimethylamino)butanoyl)-oxy)ethyl)-azanediylbis(octane 8,1-diyl) bis(2-hexyldecanoate) (EA-405). In one embodiment, the cationically ionizable lipid is (2-(4-(dimethylamino)butanoyl)-oxy)azanediylbis-(octane 8,1-diyl) bis(2-hexyldecanoate) (HY-405). In one embodiment, the cationically ionizable lipid is O—[N-{(9Z,12Z)-octadeca-9,12-dien-1-yl)}-N-{7-pentadecylcarbonyloxyoctyl}-amino]4-(dimethylamino)butanoate (HY501).

In one embodiment, the cationic or cationically ionisable lipid is present in an amount of 20 to 70 mol % of the total lipids present in the lipid mixture. In one embodiment, the cationic or cationically ionisable lipid is present in an amount of 30 to 60 mol % of the total lipids present in the lipid mixture. In one embodiment, the cationic or cationically ionisable lipid is present in an amount of 40 to 50 mol % of the total lipids present in the lipid mixture. The term “lipid mixture” in this context applies to the lipid mixture component of both the aqueous dispersion and the nucleic acid-lipid particle.

### Additional Lipids

The lipid mixture in the aqueous dispersion and lipid particles of the present invention may further comprise one or more additional lipids. In one embodiment, the one or more additional lipids comprise an anionic amphiphile, as defined and exemplified below. In one embodiment, the one or more additional lipids comprise a neutral or zwitterionic lipid, as defined and exemplified below. In one embodiment, the one or more additional lipids comprise a steroid, as defined and exemplified below. In one embodiment, the one or more additional lipids comprise a neutral lipid, as defined and exemplified below. In one embodiment, the one or more additional lipids comprise a neutral lipid (such as a steroid), as defined and exemplified below. In one embodiment, the one or more additional lipids comprise a peptide-conjugated lipid), as defined and exemplified below.

### Neutral Lipid

The composition may also additionally comprise a neutral lipid. The neutral lipid is preferably a neutral phospholipid. In one embodiment, the phospholipid may be zwitterionic (i.e. it carries both a positive and a negative charge, so that it is neutral at a pH ranging around neutral).

In some embodiments, the phospholipid is selected from the group consisting of phosphatidylcholines, phosphatidylethanolamines, and sphingomyelins. The hydrocarbyl portion of the acyl moieties of phospholipids is as defined above, but is preferably an alkyl group (as defined above) having 6 to 40, preferably 8 to 24, carbon atoms or an alkenyl group (as defined above) having 6 to 40, preferably 14 to 22, carbon atoms and 1 to 6 carbon-carbon double bonds. The acyl parts of the phospholipids may be the same or different. In one embodiment, the acyl moieties are saturated fatty acid moieties having 8 to 24 carbon atoms (including the acyl carbon), preferably selected from the group consisting of lignoceroyl, behenoyl, arachidoyl, stearoyl, palmitoyl, myristoyl, lauroyl, decanoyl and octanoyl moieties. In a specific embodiment, neutral phospholipids have a Tₘ of 30° C. or higher and are selected from di-stearoyl or di-palmitoyl or stearoyl-palmitoyl moieties. In one embodiment, the acyl moieties are unsaturated fatty acid moieties having 14 to 22 carbon atoms (including the acyl carbon), preferably selected from the group consisting of oleoyl, linoyl, and lineoyl moieties.

Examples of such phospholipids include diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine (DLPC), dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphosphatidylcholine (DLPC), palmitoyloleoylphosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-10-glycero-3-phosphocholine (C16 Lyso PC) and phosphatidylethanolamines, in particular diacylphosphatidyl-ethanolamines, such as dioleoylphosphatidylethanolamine (DOPE), distearoyl-phosphatidylethanolamine (DSPE), dipalmitoyl-phosphatidylethanolamine (DPPE), dimyristoylphosphatidylethanolamine (DMPE), dilauroyl-phosphatidylethanolamine (DLPE), diphytanoyl-phosphatidylethanolamine (DPyPE), 1,2-di-(9Z-octadecenoyl)-sn-glycero-3-phosphocholine (DOPG), 1,2-dipalmitoyl-sn-glycero-3-phospho-(1′-rac-glycerol) (DPPG), 1-palmitoyl-2-oleoyl-sn-glycero-3-phosphoethanolamine (POPE), N-palmitoyl-D-erythro-sphingosylphosphorylcholine (SM), and further phosphatidyl-ethanolamine lipids with different hydrophobic chains.

In some embodiments, the neutral lipid is selected from the group consisting of DSPC, DOPC, DMPC, DPPC, POPC, DOPE, DOPG, DOPE, and SM, or a mixture of any thereof.

Thus, in some embodiments, the lipid nanoparticle compositions described herein comprise a cationic or cationically ionizable lipid (as defined herein) and a phospholipid. In some embodiments, the lipid nanoparticle compositions described herein comprise a cationic or cationically ionizable lipid and a phospholipid selected from the group consisting of DSPC, DOPC, DMPC, DPPC, POPC, DOPE, DOPG, DOPE, and SM, or a mixture of any thereof.

In one embodiment, the neutral lipid is present in the lipid mixture in an amount of about 1 mol % to about 40 mol % of the total lipids present in the lipid mixture. In one embodiment, the neutral lipid is present in the lipid mixture in an amount of about 2 mol % to about 25 mol % of the total lipids present in the lipid mixture. In one embodiment, the neutral lipid is present in the lipid mixture in an amount of from about 5 mol % to about 15 mol % of the total lipids present in the lipid mixture.

In one embodiment, the neutral lipid is a phospholipid and is present in the lipid mixture in an amount of about 1 mol % to about 40 mol % of the total lipids present in the lipid mixture. In one embodiment, the neutral lipid is a phospholipid and is present in the lipid mixture in an amount of about 2 mol % to about 25 mol % of the total lipids present in the lipid mixture. In one embodiment, the neutral lipid is a phospholipid and is present in the lipid mixture in an amount of from about 5 mol % to about 15 mol % of the total lipids present in the lipid mixture.

In one embodiment, the neutral lipid is a phosphatidylcholine and is present in the lipid mixture in an amount of about 1 mol % to about 40 mol % of the total lipids present in the lipid mixture. In one embodiment, the neutral lipid is a phosphatidylcholine and is present in the lipid mixture in an amount of about 2 mol % to about 25 mol % of the total lipids present in the lipid mixture. In one embodiment, the neutral lipid is a phosphatidylcholine and is present in the lipid mixture in an amount of from about 5 mol % to about 15 mol % of the total lipids present in the lipid mixture.

In one embodiment, the neutral lipid is DSPC and is present in the lipid mixture in an amount of about 1 mol % to about 40 mol % of the total lipids present in the lipid mixture. In one embodiment, the neutral lipid is DSPC and is present in the lipid mixture in an amount of about 2 mol % to about 25 mol % of the total lipids present in the lipid mixture. In one embodiment, the neutral lipid is DSPC and is present in the lipid mixture in an amount of from about 5 mol % to about 15 mol % of the total lipids present in the lipid mixture.

In each of the above embodiments, the term “lipid mixture” in this context applies to the lipid mixture component of both the aqueous dispersion and the nucleic acid-lipid particle.

### Steroid

The lipid nanoparticle compositions of the present invention also comprise a steroid. In one embodiment, the steroid comprises a sterol. In one embodiment, the steroid is cholesterol.

Thus, in some embodiments, the lipid nanoparticle compositions described herein comprise a cationically ionizable lipid (as defined herein) and cholesterol.

In one embodiment, the steroid is present in an amount ranging from about 10 mol % to about 65 mol % of the total lipids present in the lipid mixture. In one embodiment, the steroid is present in an amount ranging from about 20 mol % to about 60 mol % of the total lipids present in the lipid mixture. In one embodiment, the steroid is present in an amount ranging from about 30 mol % to about 50 mol % of the total lipids present in the lipid mixture.

In some embodiments, the combined concentration of the neutral lipid (in particular, one or more phospholipids, in particular a phosphatidylcholine such as DSPC) and steroid (in particular, cholesterol) may comprise from about 0 mol % to about 70 mol %, such as from about 2 mol % to about 60 mol %, from about 5 mol % to about 55 mol %, from about 5 mol % to about 50 mol %, from of the total lipids present in the lipid mixture.

In each of the above embodiments, the term “lipid mixture” in this context applies to the lipid mixture component of both the aqueous dispersion and the nucleic acid-lipid particle.

### Anionic Amphiphile

In one embodiment, the composition of the present disclosure also includes a negatively charged amphiphile (an “anionic amphiphile”). In this specification the term “amphiphile” is defined generally as a molecule having both hydrophilic and hydrophobic moieties (as defined above). The negative charge is situated in the hydrophilic portion of the amphiphile. The negatively charged amphiphile may have one negatively charged group or multiple (e.g. 2, 3, 4, or 5) negatively charged groups. Anionic amphiphiles having a single negatively charged group are preferred.

In the present invention, the anionic amphiphile has a pH-sensitive charge. In this context a “pH-sensitive charge” means that the amphiphile carries the negative charge at alkaline or neutral pH, but may be neutral at acidic pH. In certain embodiments, the pH-sensitive charge is combined with a constitutive charge such as in organic phosphates wherein such amphiphile carries two negative charges at alkaline or neutral pH, but only a single negative charge at acidic pH. Amphiphiles carrying constitutive charged anionic moieties are typically salts of organic weak acids (i.e. organic acids which remains largely undissociated when dissolved in a solvent so that the proton is only partially transferred to the solvent molecule).

In one embodiment, the anionic amphiphile has a charged polar moiety selected from the group consisting of carboxylate or phosphate.

In one embodiment, the negatively charged amphiphile is a carboxylic acid or carboxylate (as defined above, either in its broadest aspect or a preferred aspect).

In one embodiment, the negatively charged amphiphile has a pH sensitive charge and pH sensitive anionic moiety is a carboxylic acid. One or more charged groups can be present in the amphiphile and in preferred embodiments a single charged moiety is present in an amphiphile.

The polar region of the negatively charged amphiphile may comprise additional uncharged polar moieties. Preferred uncharged polar moieties are hydroxyl or amide groups and one or more uncharged polar moieties can be present in the negatively charged amphiphile.

In one embodiment, the negatively charged amphiphile is a hemiester of a dicarboxylic acid with diacylglycerol. The hydrocarbyl portion of the acyl moieties of the diacylglycerol portion is as defined above, but is preferably an alkyl group (as defined above) having 6 to 40, preferably 14 to 22, carbon atoms or an alkenyl group (as defined above) having 6 to 40, preferably 14 to 22, carbon atoms. The acyl parts of the diacylglycerol moiety may be the same or different. In one embodiment, the acyl moieties are saturated fatty acid moieties, preferably selected from the group consisting of behenoyl, arachinoyl, stearoyl, palmitoyl, and myristoyl moieties. In one embodiment, the acyl moieties are unsaturated fatty acid moieties, preferably selected from the group consisting of oleoyl, linoyl, and lineoyl moieties. The dicarboxylic acid moiety is as defined above, and preferably has 2 to 8 carbon atoms, more preferably 2 to 6, even more preferably 2 to 4 carbon atoms. Examples of the dicarboxylic acid moiety include oxalate, malonate, succinate, glutarate, adipate, pimelate and suberate. Typical examples of such negatively charged amphiphiles include dimyristoylglyceryl hemisuccinate (DMGS), dipalmitoylglyceryl hemisuccinate (DPGS), palmitoylstearoylglyceryl hemisuccinate (PSGS), distearoylglyceryl hemisuccinate (DSGS), dioleoylglycerol hemisuccinate (DOGS), palmitoyloleoylglyceryl hemisuccinate (POGS) and homologues of any of the above thereof wherein the dicarboxylic acid portion is oxalate, malonate, succinate, glutarate, adipate, pimelate or suberate. Dimyristoylglyceryl hemisuccinate (DMGS) or dioleoylglyceryl hemisuccinate (DOGS) are preferred.

In one embodiment, the negatively charged amphiphile is a hemiester of a dicarboxylic acid with a steroid. The dicarboxylic acid moiety is as defined and exemplified above, and typically contains a total (including the acyl carbons) of 2 to 6, preferably 3 to 5, most preferred 4 carbon atoms. The ester group may, with preference esterify the 3′ hydroxyl group on the steroid molecule.

In one embodiment, the negatively charged amphiphile is a hemiester of a dicarboxylic acid with cholesterol. The dicarboxylic acid moiety is as defined above, and preferably has 2 to 6 carbon atoms, more preferably 3 to 5, even more preferably 4 carbon atoms. Examples of the dicarboxylic acid moiety include oxalate, malonate, succinate, glutarate and adipate, wherein succinate is preferred. Typical examples of such negatively charged amphiphiles include cholesteryl hemisuccinate and cholesteryl hemiadipate, of which cholesterol hemisuccinate is preferred.

In one embodiment, the negatively charged amphiphile is a monoester or diester of a phosphoric acid, wherein one of the phosphoric acid hydroxyl groups is esterified with diacylglycerol. The hydrocarbyl portion of the acyl moieties of the diacylglycerol portion is as defined above, but is preferably an alkyl group (as defined above) having 6 to 40, preferably 14 to 22, carbon atoms or an alkenyl group (as defined above) having 6 to 40, preferably 14 to 22, carbon atoms. The acyl parts of the diacylglycerol moiety may be the same or different. In one embodiment, the acyl moieties are saturated fatty acid moieties, preferably selected from the group consisting of behenoyl, arachinoyl, stearoyl, palmitoyl, myristoyl, moieties. In one embodiment, the acyl moieties are unsaturated fatty acid moieties, preferably selected from the group consisting of oleoyl, linoyl, and lineoyl moieties.

In one embodiment, the anionic amphiphile is a carboxylic acid, preferably selected from the group consisting of hexanoic acid, octanoic acid, decanoic acid, lauric acid, myristic acid, palmitic acid, stearic acid, eicosanoic acid, tricosanic acid, 2-hydroxytetradecanoic acid, 2-methyloctadecanoic acid, 2-bromohexadecanoic acid, 2-propylpentanoic acid, 2-butyloctanoic acid, 2-hexyldecanoic acid, 9-hydroxy-stearic-acid, trans-2-decenoic acid, (9Z)-9-hexadecenoic acid, linolic acid, linolenic acid, oleic acid, elaidic acid, arachidonic acid, cyclododecanoic acid, adamantylacetic acid, dicyclohexylacetic acid, trans-4-pentylcyclohexane-carboxylic acid, 4-(decyloxy)benzoic acid, 4-octylbenzoic acid, cholic acid, and lithocholic acid, or a mixture of any thereof.

In one embodiment, the anionic amphiphile is a hemiester of a dicarboxylic acid with diacylglycerol, preferably selected from the group consisting of dimyristoyl hemisuccinate and dioleoyl hemisuccinate, or a mixture of any thereof.

In one embodiment, the anionic amphiphile is a hemiester of a dicarboxylic acid with cholesterol, preferably selected from the group consisting of cholesterol hemisuccinate, cholesterol hemimalonate and cholesterol hemiadipate, or a mixture of any thereof.

In one embodiment, the anionic amphiphile is an organic sulfate or sulfonate, preferably selected from the group consisting of sodium lauryl sulfate, sodium hexadecane sulfonate and sodium dodecylbenzene sulfonate, or a mixture of any thereof.

In one embodiment, the anionic amphiphile is an organic phosphonate, preferably selected from the group consisting of octadecylphosphonic acid and dodecylphosphonic acid, or a mixture of any thereof.

In one embodiment, the anionic amphiphile is an anionic phospholipid, preferably selected from the group consisting of phosphatidylserine, phosphatidylglycerol and phosphatidic acid, or a mixture of any thereof.

In one embodiment, the anionic amphiphile is selected from the group consisting of:
a carboxylic acid;
a hemiester of a dicarboxylic acid with cholesterol;
a hemiester of a dicarboxylic acid with diacylglycerol;
a phosphate ester with diacylglycerol;
or a mixture of any thereof.

In one embodiment, the anionic amphiphile is selected from the group consisting of:
cholesterol hemisuccinate (CHEMS);
dimyristoyl hemisuccinate (DMGS);
dioleoylglycerol hemisuccinate (DOGS);
or a mixture of any thereof.

In one embodiment, the anionic amphiphile is CHEMS. In one embodiment, the anionic amphiphile is DMGS. In one embodiment, the anionic amphiphile is DOGS.

In one embodiment, the anionic amphiphile is present in an amount of 0 to 50 mol % of the total lipids present in the lipid mixture. In one embodiment, the anionic amphiphile is present in an amount of 5 to 45 mol % of the total lipids present in the lipid mixture. The term “lipid mixture” in this context applies to the lipid mixture component of both the aqueous dispersion and the nucleic acid-lipid particle.

### Grafted Lipids

The compositions described herein may also contain a grafted lipid. In the present specification the term “grafted lipid” in its broadest sense means a lipid or lipid-like material, as defined above (either in a broadest aspect or a preferred aspect) conjugated to a polymer, as defined below (either in a broadest aspect or a preferred aspect”).

A “polymer” as used herein, is given its ordinary meaning, i.e., a molecular structure comprising one or more repeat units (monomers), connected by covalent bonds. The repeat units can all be identical, or in some cases, there can be more than one type of repeat unit present within the polymer. In some cases, the polymer is biologically derived, i.e., a biopolymer such as a protein. In some cases, additional moieties can also be present in the polymer, for example targeting moieties. If more than one type of repeat unit is present within the polymer, then the polymer is said to be a “copolymer.” The repeat units forming the copolymer can be arranged in any fashion. For example, the repeat units can be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In one embodiment, the grafted lipid is capable of acting as a stealth lipid. In this specification the term “stealth lipid” means a stealth polymer (as defined below) conjugated to a lipid (as defined herein). In this specification the term “stealth polymer” means a polymer (as defined above) having the following features: (a) polar (hydrophilic) functional groups; (b) hydrogen bond acceptor groups, (c) no hydrogen bond donor groups; and (d) no net charge. In some embodiments, a stealth polymer is designed to sterically stabilize a lipid particle by forming a protective hydrophilic layer that shields the hydrophobic lipid layer. In some embodiments, a stealth polymer can reduce its association with serum proteins and/or the resulting uptake by the reticuloendothelial system when such lipid particles are administered in vivo.

In one embodiment, the grafted lipid is a polyethylene-glycol conjugated lipid (also known as a PEG-lipid or PEGylated lipid). The term “PEGylated lipid” refers to a molecule comprising both a lipid portion and a polyethylene glycol portion. PEGylated lipids are known in the art. The PEG-lipid may comprise 5-1000, 5-500, 5-100, 5-50, 8-1000, 8-500, 8-100, 8-50, 10-1000, 10-500, 10-100, or 10-50, ethylene glycol repeating units, which may be consecutive.

In some embodiments, the PEG-conjugated lipid (pegylated lipid) is a lipid having the structure of the following general formula:

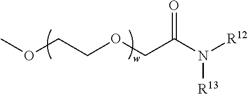

or a pharmaceutically acceptable salt, tautomer or stereoisomer thereof, wherein each of R¹² and R¹³ is each independently a straight or branched, alkyl or alkenyl chain containing from 10 to 30 carbon atoms, wherein the alkyl/alkenyl chain is optionally interrupted by one or more ester bonds; and w has a mean value ranging from 30 to 60.

In some embodiments of this formula, each of R¹² and R¹³ is independently a straight alkyl chain containing from 10 to 18 carbon atoms, preferably from 12 to 16 carbon atoms.

In some embodiments of this formula, R¹² and R¹³ are identical. In some embodiments, each of R¹² and R¹³ is a straight alkyl chain containing 12 carbon atoms. In some embodiments, each of R¹² and R¹³ is a straight alkyl chain containing 14 carbon atoms. In some embodiments, each of R¹² and R¹³ is a straight alkyl chain containing 16 carbon atoms.

In some embodiments of this formula, R¹² and R¹³ are different. In some embodiments, one of R¹² and R¹³ is a straight alkyl chain containing 12 carbon atoms and the other of R¹² and R¹³ is a straight alkyl chain containing 14 carbon atoms.

In some embodiments of this formula, w has a mean value ranging from 40 to 50, such as a mean value of 45.

In some embodiments of this formula, w is within a range such that the PEG portion of the pegylated lipid has an average molecular weight of from about 400 to about 6000 g/mol, such as from about 1000 to about 5000 g/mol, from about 1500 to about 4000 g/mol, or from about 2000 to about 3000 g/mol. In some embodiments, each of R¹² and R¹³ is a straight alkyl chain containing 14 carbon atoms and w has a mean value of 45.

Various PEG-conjugated lipids are known in the art and include, but are not limited to pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy(polyethoxy)ethyl-N-(2,3-di(tetradecanoxy)propyl)carbamate or 2,3-di(tetradecanoxy)propyl-N-(ω methoxy(polyethoxy)ethyl)carbamate, and the like. In some embodiments of this formula, the PEG portion of the pegylated lipid has an average molecular weight of from about 400 to about 6000 g/mol, such as from about 1000 to about 5000 g/mol, from about 1500 to about 4000 g/mol, or from about 1700 to about 3000 g/mol, or from about 1800 to about 2200 g/mol. In one embodiment, the PEG portion of the pegylated lipid has an average molecular weight of about 2000 g/mol.

In some embodiments, the PEG-conjugated lipid (pegylated lipid) is or comprises 2-[(polyethylene glycol)-2000]—N,N-ditetradecylacetamide. In some embodiments, the pegylated lipid has the following structure:

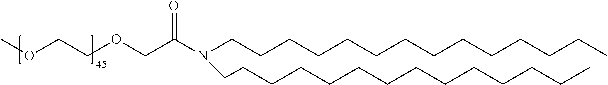

Other examples of grafted lipids include poly(sarcosine) (pSar)-conjugated lipids, poly(oxazoline) (POX)-conjugated lipids; poly(oxazine) (POZ)-conjugated lipids, poly(vinyl pyrrolidone) (PVP)-conjugated lipids; poly(N-(2-hydroxypropyl)-methacrylamide) (pHPMA)-conjugated lipids; poly(dehydroalanine) (pDha)-conjugated lipids; poly(aminoethoxy ethoxy acetic acid) (pAEEA)-conjugated lipids and poly(2-methylaminoethoxy ethoxy acetic acid) (pmAEEA)-conjugated lipids.

In one embodiment, the grafted lipid is a polysarcosine-conjugated lipid, also referred to herein as sarcosinylated lipid or pSar-lipid. The term “sarcosinylated lipid” refers to a molecule comprising both a lipid portion and a polysarcosine (poly(N-methylglycine) portion, the polysarcosine portion having the repeating unit shown below:

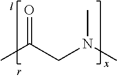

wherein x refers to the number of sarcosine units. The polysarcosine may comprise from 2 to 200, from 2 to 100, from 5 to 200, from 5 to 100, from 10 to 200, from 10 to 100, optionally from 5 to 80, preferably from 10 to 70 sarcosine units, preferably from 15 to 50 sarcosine units, more preferably from 20 to 30 sarcosine units, even more preferably 21 to 25 sarcosine units.

In one embodiment, the grafted lipid comprises a polysarcosine portion (as defined and exemplified above) the carbonyl terminus of which is bonded to a (C₆₋₃₀ alkyl)amine (as defined and exemplified above), and the amino terminus of which is optionally bonded to an acetyl group. In one embodiment, the grafted lipid comprises a polysarcosine portion (as defined and exemplified above) the carbonyl terminus of which is bonded to a (C₁₂₋₂₀ alkyl)amine (as defined and exemplified above), and the amino terminus of which is optionally bonded to an acetyl group. In one embodiment, the grafted lipid comprises a polysarcosine portion (as defined and exemplified above) the carbonyl terminus of which is bonded to a (C₁₄ alkyl)amine (as defined and exemplified above), and the amino terminus of which is optionally bonded to an acetyl group.

In one especially preferred embodiment, the grafted lipid is n-tetradecyl poly(sarcosine)23 (C14-pSar 23), having the following structure:

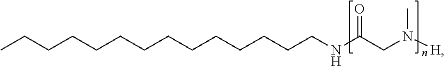

where n is 23.

In one especially preferred embodiment, the grafted lipid is n-tetradecyl poly(sarcosine)23 acetate (C14-pSar 23Ac), having the following structure:

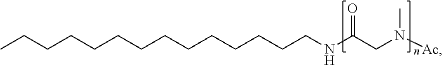

where n is 23.

In one embodiment, the grafted lipid is a polyoxazoline (POX)-conjugated and/or a polyoxazine (POZ)-conjugated lipid and/or a POX/POZ-conjugated lipid, also referred to herein as a conjugate of a POX and/or POZ polymer and one or more hydrophobic chains or as oxazolinylated and/or oxazinylated lipid or POX and/or POZ-lipid. The term “oxazolinylated lipid” or “POX-lipid” refers to a molecule comprising both a lipid portion and a polyoxazoline portion, the polyoxazoline portion (pOx) having the repeating unit shown below. The term “oxazinylated lipid” or “POZ-lipid” refers to a molecule comprising both a lipid portion and a polyoxazine portion, the polyoxazine (pOz) portion having the repeating unit shown below. The term “oxazolinylated/oxazinylated lipid” or “POX/POZ-lipid” or “POXZ-lipid” refers to a molecule comprising both a lipid portion and a portion of a copolymer of polyoxazoline and polyoxazine, i.e. a polymer having both the pOx and pOz repeating units shown below:

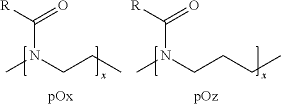

wherein x refers to the number of pOx and/or pOz units. The total number of pOx and/or pOz repeating units in the polymer may comprise from 2 to 200, from 2 to 100, from 5 to 200, from 5 to 100, from 10 to 200, from 10 to 100, optionally from 5 to 80, preferably from 10 to 70 pOx and/or pOz units.

In one embodiment, the grafted lipid is a poly(vinyl pyrrolidone) (PVP)-conjugated lipid. In one embodiment, the lipid nanoparticle composition is substantially free (as defined above, either in its broadest aspect of a preferred aspect) of a poly(vinyl pyrrolidone) (PVP) conjugated to a lipid. The term “poly(vinyl pyrrolidone)” or “PVP” means a polymer having a vinyl pyrrolidine repeating unit, i.e. the repeating unit shown below.

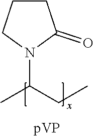

In one embodiment, the grafted lipid is a poly(N-(2-hydroxypropyl)methacrylamide) (pHIPMA)-conjugated lipid. In one embodiment, the lipid nanoparticle composition is substantially free (as defined above, either in its broadest aspect of a preferred aspect) of poly(N-(2-hydroxypropyl)methacrylamide) (pHPMA) conjugated to a lipid. The term “poly(N-(2-hydroxypropyl)-methacrylamide” or “pHPMA” means a polymer having the repeating unit shown below.

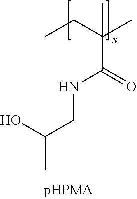

In one embodiment, the grafted lipid is a poly(dehydroalanine) (pDha)-conjugated lipid. The term “pDha” means a polymer having the repeating unit shown below.

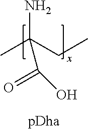

In one embodiment, the grafted lipid is an amphiphilic oligoethylene glycol (OEG)-conjugated lipid. Examples of amphiphilic oligoethylene glycol (OEG)-conjugated lipids include poly(aminoethyl-ethylene glycol acetyl) (pAEEA) and/or poly(methylaminoethyl-ethylene glycol acetyl) (pmAEEA). The terms “pAEEA” and “pmAEAA” means a polymer having the repeating unit shown below:

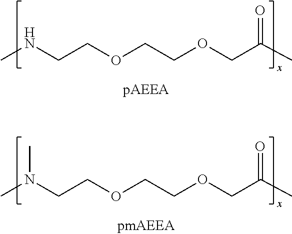

wherein x refers to the total number of pAEEA and/or pmAEEA units in the polymer. The total number of pAEEA and/or pmAEEA repeating units in the polymer may comprise from 1 to 100, from 5 to 50, from 5 to 25, from 7 to 14, preferably from 10 to 20, more preferably 12 to 16.

The lipid portion of the (pAEEA)-conjugated lipid may be any of those defined above in relation to lipids, either in a broadest aspect or a preferred aspect. In one embodiment, the lipid portion is a tocopherol or tocotrienol residue. In one embodiment, the lipid portion is α-tocopherol. In one embodiment, the lipid portion is β-tocopherol. In one embodiment, the lipid portion is γ-tocopherol. In one embodiment, the lipid portion is δ-tocopherol. In one embodiment, the lipid portion is α-tocotrienol. In one embodiment, the lipid portion is β-tocotrienol. In one embodiment, the lipid portion is γ-tocotrienol. In one embodiment, the lipid portion is δ-tocotrienol.

In one embodiment, the grafted lipid is α-tocopherol pAEEA14.

In one embodiment, the grafted lipid is a peptide-conjugated lipid. The compositions described herein may also contain a lipid conjugated to a binding moiety. In some embodiments, the lipid having a binding moiety covalently attached thereto comprises a compound L-X1-P-X2-B, as described further herein. Preferably the binding moiety is a peptide, and the compositions contain described herein may also contain a peptide-conjugated lipid. In the present specification the term “peptide-conjugated lipid” in its broadest sense means a lipid or lipid-like material, as defined above (either in a broadest aspect or a preferred aspect) conjugated to a peptide. In this aspect “peptide” is synonymous with “polypeptide” and “protein”. In one embodiment the peptide comprises an ALFA-tag, (i.e., the peptide conjugated lipid may be an ALFA-conjugated lipid). Such peptide-conjugated lipids are described in more detail in U.S. 63/305,905 (unpublished at time of filing).

In some embodiments, the peptide-conjugated lipid comprises a compound of Formula (A):

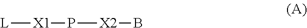

wherein
P comprises a polymer;
L comprises a hydrophobic moiety attached to a first end of the polymer;
B comprises a binding moiety attached to a second end of the polymer;
X1 is absent or a first linking moiety; and
X2 is absent or a second linking moiety.

In some embodiments, X1 comprises a carbonyl group.

In some embodiments, X2 comprises the reaction product of a maleimide group with a thiol or cysteine group of a compound comprising the binding moiety.

In some embodiments, the hydrophobic moiety is or is comprised in a lipid. In some embodiments, the lipid comprises a phospholipid, e.g., 1,2-distearoyl-sn-glycero-3-phosphoethanolamine (DSPE).

In some embodiments, the polymer provides stealth property, extends circulation half-life and/or reduces non-specific protein binding or cell adhesion.

In some embodiments, the polymer comprises polyethylene glycol (PEG). The average molecular weight of the PEG may range from 200 to 10,000, preferably 500 to 5000, more preferably 1000 to 4000, most preferably 2000.

In some embodiments, the hydrophobic moiety having a binding moiety covalently attached thereto comprises a distearoyl-glycero-phosphoethanolamine-polyethylene glycol-conjugate (DSPE-PEG).

In some embodiments, the binding moiety covalently attached to the hydrophobic moiety comprises a peptide, preferably the binding moiety comprises an ALFA-tag.

In some embodiments, an ALFA-tag comprises the amino acid sequence -AA0-AA1-AA2-AA3-AA4-AA5-AA6-AA7-AA8-AA9-AA10-AA11-AA12-AA13-AA14-, wherein the amino acids of AA0, AA1, AA2, AA3, AA4, AA5, AA6, AA7, AA8, AA9, AA10, AA11, AA12, AA13 and AA14 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, an ALFA-tag comprises a sequence selected from the group consisting of SRLEEELRRRLTE, PSRLEEELRRRLTE, SRLEEELRRRLTEP, and PSRLEEELRRRLTEP.

In some embodiments, an ALFA-tag comprises the cyclized amino acid sequence -AA0-AA1-AA2-AA3-AA4-AA5-AA6-AA7-AA8-AA9-AA10-AA11-AA12-AA13-AA14-, wherein the side-chains of any two of the amino acids of AA0, AA1, AA2, AA3, AA4, AA5, AA6, AA7, AA8, AA9, AA10, AA11, AA12, AA13 and AA14 (X1, X2) are connected covalently; and wherein the amino acids of AA0, AA1, AA2, AA3, AA4, AA5, AA6, AA7, AA8, AA9, AA10, AA11, AA12, AA13 and AA14 which are not X1 and X2 are:
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA10 is Arg;
AA11 is Leu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X1 and X2 are separated by 2 or 3 amino acids.

In some embodiments, AA5 is X1 and AA9 is X2, AA5 is X1 and AA8 is X2, AA9 is X1 and AA13 is X2, AA6 is X1 and AA9 is X2, AA9 is X1 and AA12 is X2, AA10 is X1 and AA13 is X2, AA6 is X1 and AA10 is X2 or AA4 is X1 and AA8 is X2.

In some embodiments, an ALFA-tag comprises a cyclized amino acid sequence selected from the group consisting of
a. -AA0-AA1-AA2-AA3-AA4-cyclo(X1-AA6-AA7-AA8-X2)-Arg-Leu-AA12-AA13-AA14-,
b. -AA0-AA1-AA2-AA3-AA4-cyclo(X1-AA6-AA7-X2)-AA9-Arg-Leu-AA12-AA13-AA14-,
c. -AA0-AA1-AA2-AA3-AA4-AA5-AA6-AA7-AA8-cyclo(X1-Arg-Leu-AA12-X2)-AA14-,
d. -AA0-AA1-AA2-AA3-AA4-AA5-cyclo(X1-AA7-AA8-X2)-Arg-Leu-AA12-AA13-AA14-,
e. -AA0-AA1-AA2-AA3-AA4-AA5-AA6-AA7-AA8-cyclo(X1-Arg-Leu-X2)-AA13-AA14-,
f. -AA0-AA1-AA2-AA3-AA4-AA5-AA6-AA7-AA8-AA9-cyclo(X1-Leu-AA12-X2)-AA14-,
g. -AA0-AA1-AA2-AA3-AA4-AA5-cyclo(X1-AA7-AA8-AA9-X2)-Leu-AA12-AA13-AA14-, and
h. -AA0-AA1-AA2-AA3-cyclo(X1-AA5-AA6-AA7-X2)-AA9-Arg-Leu-AA12-AA13-AA14-,
wherein the side-chains of X1 and X2 amino acid residues are connected covalently;
AA0 is Pro or deleted;
AA1 is Ser, Gly, Thr, or Pro;
AA2 is Arg, Gly, Ala, Glu, or Pro;
AA3 is Leu, Ile, or Val;
AA4 is Glu or Gln;
AA5 is Glu or Gln;
AA6 is Glu or Gln;
AA7 is Leu, Ile, or Val;
AA8 is Arg, Ala, Gln, or Glu;
AA9 is Arg, Ala, Gln, or Glu;
AA12 is Thr, Ser, Asp, Glu, Pro, Ala, or deleted;
AA13 is Glu, Lys, Pro, Ser, Ala, Asp, or deleted; and
AA14 is Pro or deleted.

In some embodiments, X1 and X2 in the peptides disclosed herein are connected covalently via an amide, disulfide, thioether, ether, ester, thioester, thioamide, alkylene, alkenylene, alkynylene, and/or 1,2,3-triazole.

In some embodiments, a cyclized amino acid sequence described herein is generated by linking an amino group of a side-chain of one of X1 and X2 to the carboxyl group of a side-chain of the other of X1 and X2 via an amide bond. The amino group of the side chain of an amino acid that possesses a pendant amine group, e.g., lysine or a lysine derivative, and the carboxyl group of the side chain of an acidic amino acid, e.g., aspartic acid, glutamic acid or a derivative thereof, can be used to generate a cyclized amino acid sequence via an amide bond.

In some embodiments, a cyclized amino acid sequence described herein is generated by linking a sulfhydryl group of a side-chain of one of X1 and X2 to the sulfhydryl group of a side-chain of the other of X1 and X2 via a disulfide bond. Sulfhydryl group-containing amino acids include cysteine and other sulfhydryl-containing amino acids as Pen.

In some embodiments, X1 and X2 are, independently, selected from the group consisting of Glu, DGlu, Asp, DAsp, Lys, DLys, hLys, DhLys, Orn, DOrn, Dab, DDab, Dap, DDap, Cys, DCys, hCys, DhCys, Pen, and DPen, with the proviso that when X1 is Glu, DGlu, Asp, or DAsp, X2 is Lys, DLys, hLys, DhLys, Orn, DOrn, Dab, DDab, Dap, or DDap; when X1 is Lys, DLys, hLys, DhLys, Orn, DOrn, Dab, DDab, Dap, or DDap, X2 is Glu, DGlu, Asp, or DAsp; and when X1 is Cys, DCys, hCys, DhCys, Pen, or DPen, X2 is Cys, DCys, hCys, DhCys, Pen, or DPen.

In some embodiments, X1 is Glu and X2 is Lys. In some embodiments, -cyclo(Glu--------Lys)-, -c(Glu-------Lys)-, -cyclo(E-------K)-, -c(E---------K)-, -E------K-cyclo, or -cycloE----cycloK comprises the following structure:

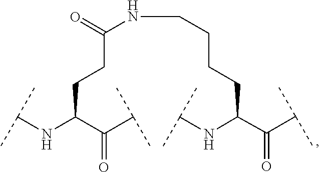

In some embodiments, X1 is Lys and X2 is Glu. In some embodiments, -cyclo(Lys------Glu)-, -c(Lys------Glu)-, -cyclo(K------E)-, -c(K-------E)-, -K-----E-cyclo, or cycloK------cycloE comprises the following structure:

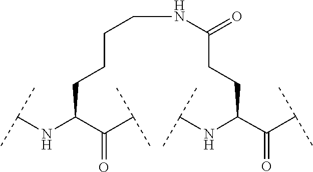

In some embodiments, X1 is Cys and X2 is Cys. In some embodiments, -cyclo(Cys------Cys)-, c(Cys------Cys)-, -cyclo(C------C)-, -c(C--------C)-, -C----C-cyclo, or -cycloC-----cycloC comprises the following structure:

In some embodiments, the cyclized amino acid sequence is -Ser-Arg-Leu-Glu-cyclo(Glu-Glu-Leu-Arg-Lys)-Arg-Leu-Thr-Glu-. In some other embodiments, the cyclized amino acid sequence is -Ser-Arg-Leu-Glu-cyclo(Asp-Glu-Leu-Arg-Lys)-Arg-Leu-Thr-Glu-. In yet some other embodiments, the cyclized amino acid sequence is -Ser-Arg-Leu-Glu-cyclo(Glu-Glu-Leu-Lys)-Arg-Arg-Leu-Thr-Glu-. In still some other embodiments, the cyclized amino acid sequence is -Ser-Arg-Leu-Glu-Glu-Glu-Leu-Arg-cyclo(Lys-Arg-Leu-Thr-Glu)-.

The cyclic peptides may have different cyclic bridging moieties forming the ring structure. Preferably, chemically stable bridging moieties are included in the ring structure such as, for example, an amide group, a lactone group, an ether group, a thioether group, a disulfide group, an alkylene group, an alkenyl group, or a 1,2,3-triazole. The following are examples illustrating the variability of bridging moieties in a peptide:

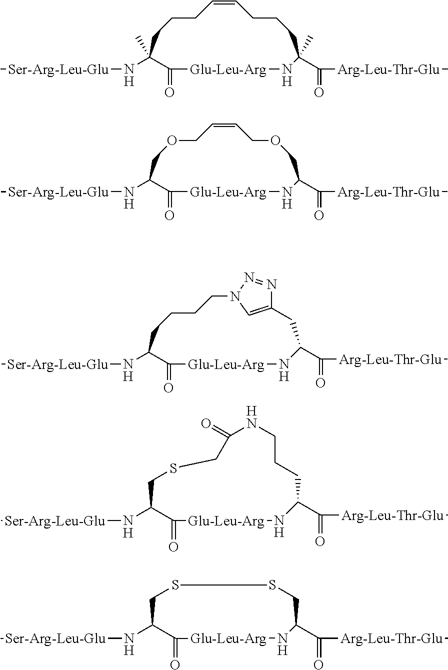

The peptide-conjugated lipid may be comprised in the lipid mixture as described herein, as incorporated into the aqueous dispersion. The peptide-conjugated lipid may not be comprised in the lipid mixture, and may instead be subsequently added to the lipid particles comprised in the dispersed phase of the aqueous dispersion. The peptide-conjugated lipid may not be comprised in the lipid mixture, and may instead be subsequently added to the nucleic acid-lipid particles. Where the peptide-conjugated lipid is added to the lipid particles comprised in the dispersed phase of the aqueous dispersion or to the nucleic acid-lipid particles, the amount of peptide-conjugated lipid added may displace the corresponding amount of steroid (e.g., cholesterol) in the particle. The peptide-conjugated lipid is typically added to the particle at a final molar ratio of 0.1-0.3 mol %, optionally about 0.2 mol %, of the total lipid.

When the nucleic acid-lipid particles comprise peptide-conjugated lipids, this allows for functionalization of the nucleic acid-lipid particles. For example, a binding moiety that specifically binds to the peptide of the peptide-conjugated lipid may be bound to the nucleic acid-lipid particles, wherein the binding moiety may also bind to target cells (for example by specifically binding a target cell surface antigen). This may provide for targeted delivery of the nucleic acid comprised within the functionalized nucleic acid-lipid particles. The binding moiety that specifically binds to the peptide of the peptide-conjugated lipid may be an ALFA-tag binding moiety.

In some embodiments, an ALFA-tag binding moiety comprises an antibody or antibody fragment, e.g., a camelid VHH domain. In some embodiments, an ALFA-tag binding moiety comprises a single-domain antibody (sdAb), NbALFA-nanobody. In some embodiments, an ALFA-tag binding moiety comprises a single domain antibody, e.g., a camelid VHH domain comprising the CDR1 sequence VTXISALNAMAMG, wherein X1 is I or V, the CDR2 sequence AVSX2RGNAM, wherein X2 is E, H, N, D, or S, and the CDR3 sequence LEDRVDSFHDY.

In some embodiments, an ALFA-tag binding moiety comprises a single domain antibody, e.g., a camelid VHH domain comprising the CDR1 sequence GVTXl SALNAMAMG, wherein X1 is I or V, the CDR2 sequence AVSX2RGNAM, wherein X2 is E, H, N, D, or S, and the CDR3 sequence LEDRVDSFHDY.

In some embodiments, an ALFA-tag binding moiety comprises a single domain antibody, e.g., a camelid VHH domain comprising the amino acid sequence EVQLQESGGGLVQPGGSLRLSCTASGVTISALNAMAMGWYRQAPGERRVMV AAVSERGNAMYRESVQGRFTVTRDFTNKMVSLQMDNLKPEDTAVYYCHVL EDRVDSFHDYWGQGTQVTVSS, an amino acid sequence having at least 99%, 98%, 97%, 96%, 95%, 90%, 85%, or 80% identity to said amino acid sequence, or a fragment of said amino acid sequence or the amino acid sequence having at least 99%, 98%, 97%, 96%, 95%, 90%, 85%, or 80% identity to said amino acid sequence. In some embodiments, the amino acid sequence comprises CDR1, CDR2 and CDR3 sequences as described above.

In some embodiments, an ALFA-tag binding moiety comprises a bispecific antibody which targets ALFA-tag and a cell surface antigen. In some embodiments, an ALFA-tag binding moiety comprises a moiety binding to a peptide comprising an ALFA-tag and a moiety targeting a cell surface antigen.

In one embodiment, the grafted lipid is present in the lipid mixture in an amount of 0.5 to 10 mol % of the total lipids present in the lipid mixture. In one embodiment, the grafted lipid is present in the lipid mixture in an amount of 0.2 to 5 mol % of the total lipids present in the lipid mixture. In one embodiment, the grafted lipid is present in the lipid mixture in an amount of 1 to 2.5 mol % of the total lipids present in the lipid mixture. The grafted lipid may comprise a mixture of (i) a grafted lipid selected from the group consisting of pSar-conjugated lipids; POX-conjugated lipids; POZ-conjugated lipids, PVP-conjugated lipids; pHIPMA-conjugated lipids; pDha-conjugated lipids; pAEEA-conjugated lipids and pmAEEA-conjugated lipids, and (ii) a peptide conjugated lipid. The term “lipid mixture” in this context applies to the lipid mixture component of both the aqueous dispersion and the nucleic acid-lipid particle.

In one embodiment, the grafted lipid is a PEG-conjugated lipid and is present in the lipid mixture in an amount of 0.5 to 10 mol % of the total lipids present in the lipid mixture. In one embodiment, the grafted lipid is a PEG-conjugated lipid and present in the lipid mixture in an amount of 0.2 to 5 mol % of the total lipids present in the lipid mixture. In one embodiment, the grafted lipid is a PEG-conjugated lipid and is present in the lipid mixture in an amount of 1 to 2.5 mol % of the total lipids present in the lipid mixture. In one embodiment, the grafted lipid is a PEG-conjugated lipid and is present in the lipid mixture in an amount of about 1.8 mol % of the total lipids present in the lipid mixture. The term “lipid mixture” in this context applies to the lipid mixture component of both the aqueous dispersion (typically containing pre-LNPs) and the nucleic acid-lipid particle.

In one embodiment, the grafted lipid is ALC-0159 and is present in the lipid mixture in an amount of 0.5 to 10 mol % of the total lipids present in the lipid mixture. In one embodiment, the grafted lipid is ALC-0159 and present in the lipid mixture in an amount of 0.2 to 5 mol % of the total lipids present in the lipid mixture. In one embodiment, the grafted lipid is ALC-0159 and is present in the lipid mixture in an amount of 1 to 2.5 mol % of the total lipids present in the lipid mixture. In one embodiment, the grafted lipid is ALC-0159 and is present in the lipid mixture in an amount of about 1.8 mol % of the total lipids present in the lipid mixture. The term “lipid mixture” in this context applies to the lipid mixture component of both the aqueous dispersion (typically containing pre-LNPs) and the nucleic acid-lipid particle.

In one embodiment, the grafted lipid is a poly(sarcosine) (pSar)-conjugated lipid and is present in the lipid mixture in an amount of 0.5 to 10 mol % of the total lipids present in the lipid mixture. In one embodiment, the grafted lipid is a poly(sarcosine) (pSar)-conjugated lipid and is present in the lipid mixture in an amount of 0.2 to 5 mol % of the total lipids present in the lipid mixture. In one embodiment, the grafted lipid is a poly(sarcosine) (pSar)-conjugated lipid and is present in the lipid mixture in an amount of 1 to 2.5 mol % of the total lipids present in the lipid mixture. In one embodiment, the grafted lipid is a poly(sarcosine) (pSar)-conjugated lipid and is present in the lipid mixture in an amount of about 1.8 mol % of the total lipids present in the lipid mixture. In one embodiment, the grafted lipid is a poly(sarcosine) (pSar)-conjugated lipid and is present in the lipid mixture in an amount of 3 to 5 mol % of the total lipids present in the lipid mixture. In one embodiment, the grafted lipid is a poly(sarcosine) (pSar)-conjugated lipid and is present in the lipid mixture in an amount of about 4 mol % of the total lipids present in the lipid mixture. The term “lipid mixture” in this context applies to the lipid mixture component of both the aqueous dispersion (typically containing pre-LNPs) and the nucleic acid-lipid particle.

In one embodiment, the grafted lipid is n-tetradecyl poly(sarcosine)23 (C14-pSar 23) or n-tetradecyl poly(sarcosine)23 (C14-pSar 23) acetate, and is present in the lipid mixture in an amount of 0.5 to 10 mol % of the total lipids present in the lipid mixture. In one embodiment, the grafted lipid is n-tetradecyl poly(sarcosine)23 (C14-pSar 23) or n-tetradecyl poly(sarcosine)23 (C14-pSar 23) acetate, and is present in the lipid mixture in an amount of 0.2 to 5 mol % of the total lipids present in the lipid mixture. In one embodiment, the grafted lipid is n-tetradecyl poly(sarcosine)23 (C14-pSar 23) or n-tetradecyl poly(sarcosine)23 (C14-pSar 23) acetate, and is present in the lipid mixture in an amount of 1 to 2.5 mol % of the total lipids present in the lipid mixture. In one embodiment, the grafted lipid is n-tetradecyl poly(sarcosine)23 (C14-pSar 23) or n-tetradecyl poly(sarcosine)23 (C14-pSar 23) acetate, and is present in the lipid mixture in an amount of about 1.8 mol % of the total lipids present in the lipid mixture. In one embodiment, the grafted lipid is n-tetradecyl poly(sarcosine)23 (C14-pSar 23) or n-tetradecyl poly(sarcosine)23 (C14-pSar 23) acetate, and is present in the lipid mixture in an amount of 3 to 5 mol % of the total lipids present in the lipid mixture. In one embodiment, the grafted lipid is n-tetradecyl poly(sarcosine)23 (C14-pSar 23) or n-tetradecyl poly(sarcosine)23 (C14-pSar 23) acetate, and is present in the lipid mixture in an amount of about 4 mol % of the total lipids present in the lipid mixture. The term “lipid mixture” in this context applies to the lipid mixture component of both the aqueous dispersion (typically containing pre-LNPs) and the nucleic acid-lipid particle.

In one embodiment, the grafted lipid is a poly(aminoethyl-ethylene glycol acetyl) (pAEEA)-conjugated lipid and is present in the lipid mixture in an amount of 0.5 to 10 mol % of the total lipids present in the lipid mixture. In one embodiment, the grafted lipid is a poly(aminoethyl-ethylene glycol acetyl) (pAEEA)-conjugated lipid and is present in the lipid mixture in an amount of 0.2 to 5 mol % of the total lipids present in the lipid mixture. In one embodiment, the grafted lipid is a poly(aminoethyl-ethylene glycol acetyl) (pAEEA)-conjugated lipid and is present in the lipid mixture in an amount of 1 to 2.5 mol % of the total lipids present in the lipid mixture. In one embodiment, the grafted lipid is a poly(aminoethyl-ethylene glycol acetyl) (pAEEA)-conjugated lipid and is present in the lipid mixture in an amount of 1.8 to 2 mol % of the total lipids present in the lipid mixture.

In one embodiment, the grafted lipid is α-tocopherol-pAEEA14 and is present in the lipid mixture in an amount of 0.5 to 10 mol % of the total lipids present in the lipid mixture. In one embodiment, the grafted lipid is α-tocopherol-pAEEA14 and is present in the lipid mixture in an amount of 0.2 to 5 mol % of the total lipids present in the lipid mixture. In one embodiment, the grafted lipid is α-tocopherol-pAEEA14 and is present in the lipid mixture in an amount of 1 to 2.5 mol % of the total lipids present in the lipid mixture. In one embodiment, the grafted lipid is α-tocopherol-pAEEA14 and is present in the lipid mixture in an amount of 1.8 to 2 mol % of the total lipids present in the lipid mixture. The term “lipid mixture” in this context applies to the lipid mixture component of both the aqueous dispersion (typically containing pre-LNPs) and the nucleic acid-lipid particle.

### Pharmaceutical Compositions

The nucleic acid-lipid particle compositions described herein are useful as or for preparing pharmaceutical compositions or medicaments for therapeutic or prophylactic treatments.

The nucleic acid-lipid particle compositions described herein may be administered in the form of any suitable pharmaceutical composition.

The term “pharmaceutical composition” relates to a composition comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. In some embodiments, the therapeutically effective agent is or comprises the active ingredient, as described herein. In the context of the present disclosure, the pharmaceutical composition comprises a nucleic acid as described herein. In some embodiments, the therapeutically effective agent is or comprises a nucleic acid, as described in the present disclosure, which comprises a nucleic acid sequence (e.g., an ORF) encoding one or more polypeptides, e.g., a peptide or protein, preferably a pharmaceutically active peptide or protein.

In some embodiments, when the nucleic acid is mRNA, the mRNA integrity of the initial pharmaceutical composition (i.e., after its preparation, but before freezing, lyophilizing or storing) is at least 50%, preferably at least 60%, more preferred at least 70%, and most preferred at least 80%, such as at least 90%.

In some embodiments, the size (Z_{average}) of the particles of the initial pharmaceutical composition (i.e., after its preparation, but before freezing, lyophilizing or storing) is between about 50 nm and about 500 nm, preferably between about 40 nm and about 200 nm, more preferably between about 40 nm and about 120 nm.

In some embodiments, the polydispersity index (PDI) of the particles of the initial pharmaceutical composition (i.e., after preparation, but before freezing, lyophilizing or storing) is less than 0.3, preferably less than 0.2, more preferably less than 0.1.

The pharmaceutical compositions of the present disclosure may be in in a frozen form or in a “ready-to-use form” (i.e., in a form, in particular a liquid form, which can be immediately administered to a subject, e.g., without any processing such as thawing, reconstituting or diluting). Thus, prior to administration of a storable form of a pharmaceutical composition, this storable form has to be processed or transferred into a ready-to-use or administrable form. E.g., a frozen pharmaceutical composition has to be thawed. Ready to use injectables can be presented in containers such as vials, ampoules or syringes wherein the container may contain one or more doses.

In one embodiment, the pharmaceutical composition is lyophilized. In one embodiment, the pharmaceutical composition is spray dried. These techniques are well known to those skilled in the art.

In some embodiments, the pharmaceutical composition is in frozen form and can be stored at a temperature of about −90° C. or higher, such as about −90° C. to about −10° C. For example, the frozen pharmaceutical compositions described herein can be stored at a temperature ranging from about −90° C. to about −10° C., such as from about −90° C. to about −40° C. or from about −40° C. to about −25° C., or from about −25° C. to about −10° C., or a temperature of about −20° C.

In some embodiments of the pharmaceutical compositions in frozen form, the pharmaceutical composition can be stored for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks. For example, the frozen pharmaceutical composition can be stored for at least 4 weeks, preferably at least 1 month, more preferably at least 2 months, more preferably at least 3 months, more preferably at least 6 months at −20° C.

In some embodiments of the pharmaceutical compositions in frozen form, when the nucleic acid is mRNA, the mRNA integrity after thawing the frozen pharmaceutical composition is at least 90%, at least 95%, at least 97%, at least 98%, or substantially 100% of the initial mRNA integrity, e.g., after thawing the frozen composition which has been stored (for at least 1 week, such as at least 2 weeks, at least 3 weeks, at least 4 weeks, at least 1 month, at least 2 months, at least 3 months, at least 6 months, at least 12 months, at least 24 months, or at least 36 months, preferably at least 4 weeks) at −20° C.

In some embodiments of the pharmaceutical compositions in frozen form, the size (Z_{average}) and/or size distribution and/or PDI of the particles after thawing the frozen pharmaceutical composition is essentially equal to the size (Z_{average}) and/or size distribution and/or PDI of the particles of the initial pharmaceutical composition before freezing. For example, if a ready-to-use pharmaceutical composition is prepared from a frozen pharmaceutical composition as described herein, it is preferred that the size (Z_{average}) and/or size distribution and/or PDI of the particles contained in the ready-to-use pharmaceutical composition is essentially equal to the initial size (Z_{average}) and/or size distribution and/or PDI of the particles contained in the frozen pharmaceutical composition before freezing.

In some embodiments, when the nucleic acid is mRNA, the size of the mRNA particles and the mRNA integrity of the pharmaceutical composition after one freeze/thaw cycle, preferably after two freeze/thaw cycles, more preferably after three freeze/thaw cycles, more preferably after four freeze/thaw cycles, more preferably after five freeze/thaw cycles or more, are essentially equal to the size of the mRNA particles and the mRNA integrity of the initial pharmaceutical composition (i.e., before the pharmaceutical composition has been frozen for the first time).

In some embodiments, the pharmaceutical composition is in liquid form and can be stored at a temperature ranging from about 0° C. to about 20° C. For example, the liquid pharmaceutical compositions described herein can be stored at a temperature ranging from about 1° C. to about 15° C., such as from about 2° C. to about 10° C., or from about 2° C. to about 8° C., or at a temperature of about 5° C.

In some embodiments, when the nucleic acid is mRNA, the mRNA integrity of the pharmaceutical composition when stored is at least 70%, preferably at least 80%, more preferably at least 90%, of the initial mRNA integrity (i.e., the mRNA integrity of the initial pharmaceutical composition).

In some embodiments of the pharmaceutical compositions in liquid form, the pharmaceutical composition can be stored for at least 1 week, such as at least 2 weeks, at least 3 weeks, at least 4 weeks, at least 1 month, at least 2 months, at least 3 months, at least 6 months, at least 12 months, or at least 24 months, preferably at least 4 weeks. For example, the liquid pharmaceutical composition can be stored for at least 4 weeks, preferably at least 1 month, more preferably at least 2 months, more preferably at least 3 months, more preferably at least 6 months at 5° C.

In some embodiments of the pharmaceutical composition in liquid form, when the nucleic acid is mRNA, the mRNA integrity of the liquid composition, when stored, e.g., at 0° C. or higher for at least one week, is such that the desired effect, e.g., to induce an immune response, can be achieved. For example, the mRNA integrity of the liquid composition, when stored, e.g., at 0° C. or higher for at least one week (such as for at least 2 weeks, at least three weeks, at least four weeks, at least one month, at least two months, at least three months, at least 4 months, or at least 6 months), may be at least 90%, compared to the mRNA integrity of the initial composition, i.e., the mRNA integrity before the composition has been stored. In some embodiments, the mRNA integrity of the composition after storage for at least four weeks (e.g., for at least three months), preferably at a temperature of 0° C. or higher, such as about 2° C. to about 8° C., is at least 90%, compared to the mRNA integrity before storage.

In some embodiments, when the nucleic acid is mRNA, the initial mRNA integrity of the pharmaceutical composition (i.e., after its preparation but before storage) is at least 50% and the mRNA integrity of the pharmaceutical composition after storage for at least one week (such as for at least 2 weeks, at least three weeks, at least four weeks, at least one month, at least two months, or at least 3 months), preferably at a temperature of 0° C. or higher, such as about 2° C. to about 8° C., is at least 90% of the initial mRNA integrity.

In some embodiments of the pharmaceutical composition in liquid form, the size (Z_{average}) (and/or size distribution and/or polydispersity index (PDI)) of the particles of the pharmaceutical composition, when stored, e.g., at 0° C. or higher for at least one week, is such that the desired effect, e.g., to induce an immune response, can be achieved. For example, the size (Z_{average}) (and/or size distribution and/or polydispersity index (PDI)) of the particles of the pharmaceutical composition, when stored, e.g., at 0° C. or higher for at least one week, is essentially equal to the size (Z_{average}) (and/or size distribution and/or PDI) of the particles of the initial pharmaceutical composition, i.e., before storage.

In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm. In some embodiments, the PDI of the particles after storage of the pharmaceutical composition, e.g., at 0° C. or higher for at least one week is less than 0.3, preferably less than 0.2, more preferably less than 0.1.

In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm, and the size (Z_{average}) (and/or size distribution and/or PDI) of the particles after storage of the pharmaceutical composition, e.g., at 0° C. or higher for at least one week is essentially equal to the size (Z_{average}) (and/or size distribution and/or PDI) of the particles before storage. In some embodiments, the size (Z_{average}) of the particles after storage of the pharmaceutical composition, e.g., at 0° C. or higher for at least one week is between about 50 nm and about 500 nm, preferably between about 40 nm and about 200 nm, more preferably between about 40 nm and about 120 nm, and the PDI of the particles after storage of the pharmaceutical composition, e.g., at 0° C. or higher for at least one week is less than 0.3 (preferably less than 0.2, more preferably less than 0.1).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the particles or pharmaceutical compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or pharmaceutical compositions described herein may depend on various of such parameters. In the case that a reaction in a patient is insufficient with an initial dose, higher doses (or effectively higher doses achieved by a different, more localized route of administration) may be used.

In particular embodiments, a pharmaceutical composition of the present disclosure (e.g., an immunogenic composition, i.e., a pharmaceutical composition which can be used for inducing an immune response) is formulated as a single-dose in a container, e.g., a vial. In some embodiments, the immunogenic composition is formulated as a multi-dose formulation in a vial. In some embodiments, the multi-dose formulation includes at least 2 doses per vial. In some embodiments, the multi-dose formulation includes a total of 2-20 doses per vial, such as, for example, 2, 3, 4, 5, 6, 7, 8, 9, 10, 11, or 12 doses per vial. In some embodiments, each dose in the vial is equal in volume. In some embodiments, a first dose is a different volume than a subsequent dose.

A “stable” multi-dose formulation preferably exhibits no unacceptable levels of microbial growth, and substantially no or no breakdown or degradation of the active biological molecule component(s). As used herein, a “stable” immunogenic composition includes a formulation that remains capable of eliciting a desired immunologic response when administered to a subject.

The pharmaceutical compositions of the present disclosure may contain buffers (in particular, derived from the nucleic acid (such as RNA) compositions with which the pharmaceutical compositions have been prepared), preservatives, and optionally other therapeutic agents. In one embodiment, the pharmaceutical compositions of the present disclosure, in particular the ready-to-use pharmaceutical compositions, comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavouring agents, or colorants.

The term “diluent” relates a diluting and/or thinning agent. Moreover, the term “diluent” includes any one or more of fluid, liquid or solid suspension and/or mixing media. Examples of suitable diluents include ethanol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, the compositions described herein, such as the pharmaceutical compositions or ready-to-use pharmaceutical compositions described herein, may be administered intravenously, intraarterially, subcutaneously, intradermally, dermally, intranodally, intramuscularly or intratumourally. In certain embodiments, the (pharmaceutical) composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the (pharmaceutical) compositions, in particular the ready-to-use pharmaceutical compositions, are formulated for systemic administration. In another preferred embodiment, the systemic administration is by intravenous administration. In another preferred embodiment, the (pharmaceutical) compositions, in particular the ready-to-use pharmaceutical compositions, are formulated for intramuscular administration.

### Medical Uses and Methods of Treatment

The nucleic acid-lipid particles and pharmaceutical compositions comprising them as described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment or prevention of a viral disease caused by said virus. Provision of a tumour antigen or epitope may be useful in the treatment of a cancer disease wherein cancer cells express said tumour antigen. Provision of a functional protein or enzyme may be useful in the treatment of genetic disorder characterized by a dysfunctional protein, for example in lysosomal storage diseases (e.g. mucopolysaccharidoses) or factor deficiencies. Provision of a cytokine or a cytokine-fusion may be useful to modulate tumour microenvironment.

Therefore, in one aspect there is disclosed the nucleic acid-lipid particle, or pharmaceutical composition as defined herein, for use in medicine.

In one embodiment, there is provided a nucleic acid-lipid particle, or pharmaceutical composition as defined herein for use in delivery of a nucleic acid (such as an mRNA) to a cell. In one embodiment, there is provided a nucleic acid-lipid particle, or pharmaceutical composition as defined herein, for use in transfecting a cell with a nucleic acid (such as an mRNA). In one embodiment, there is provided use of a nucleic acid-lipid particle, or pharmaceutical composition as defined herein in the manufacture of a medicament for delivery of a nucleic acid (such as an mRNA) to a cell. In one embodiment, there is provided use of a nucleic acid-lipid particle, or pharmaceutical composition as defined herein in the manufacture of a medicament for transfecting a cell with a nucleic acid (such as an mRNA). In one embodiment, there is provided a method of delivery of a nucleic acid (such as an mRNA) to a cell, the method comprising administering to the cell the nucleic acid-lipid particle, or pharmaceutical composition as defined herein. In one embodiment, there is provided a method of transfecting a cell with a nucleic acid (such as an mRNA), the method comprising adding to the cell the nucleic acid-lipid particle, or pharmaceutical composition as defined herein; and incubating the mixture of the composition and cells for a sufficient amount of time. In some embodiments, in particular those where the nucleic acid (such as an mRNA) encodes a pharmaceutically active protein, the mixture of the composition and cells is incubated for a time sufficient to allow the expression of the pharmaceutically active protein. In some embodiments, the sufficient amount of time is at least one hour (such at least about 2 hours, at least about 3 hours, at least about 4 hours, at least about 5 hours, at least about 6 hours, at least about 9 hours, at least about 12 hours) and/or up to about 48 hours (such as up to about 36 or up to about 24 hours). In some embodiments, incubating the mixture of the composition and cells is conducted in the presence of serum (such as human serum).

The cell may be any cell capable of receiving nucleic acid (such as an mRNA) to produce a therapeutic effect. In one embodiment, the cell is a liver cell. In one embodiment, the cell is a spleen cell. In one embodiment, the cell is a lung cell.

In one embodiment, there is provided a nucleic acid-lipid particle or a pharmaceutical composition as defined herein, for use in treating a disease treatable by a nucleic acid (such as an mRNA). In one embodiment, there is provided use of a composition as defined herein, in the manufacture of a medicament for treating a disease treatable by a nucleic acid (such as an mRNA). In one embodiment, there is provided a method of treating a disease treatable by a nucleic acid (such as an mRNA) in a subject in need thereof, the method comprising administering to the subject a nucleic acid-lipid particle or a pharmaceutical composition as defined herein.

In one embodiment, there is provided a nucleic acid-lipid particle or a pharmaceutical composition as defined herein for use in a prophylactic and/or therapeutic treatment of a disease involving an antigen. In one embodiment, there is provided use of a nucleic acid-lipid particle or a pharmaceutical composition as defined herein in the manufacture of a medicament for a prophylactic and/or therapeutic treatment of a disease involving an antigen. In one embodiment, there is provided a method of prophylactic and/or therapeutic treatment of a disease involving an antigen in a subject in need thereof, the method comprising administering to the subject a nucleic acid-lipid particle or a pharmaceutical composition as defined herein.

In one embodiment, there is provided a nucleic acid-lipid particle or a pharmaceutical composition as defined herein for use in inducing an immune response. In one embodiment, there is provided use of a nucleic acid-lipid particle or a pharmaceutical composition as defined herein, in the manufacture of a medicament for inducing an immune response.

In one embodiment, there is provided a nucleic acid-lipid particle or a pharmaceutical composition as defined herein, for use in treating cancer. In one embodiment, there is provided use of a nucleic acid-lipid particle or a pharmaceutical composition as defined herein, in the manufacture of a medicament for treating cancer. In one embodiment, there is provided a method of treating cancer in a subject in need thereof, the method comprising administering to the subject a nucleic acid-lipid particle or a pharmaceutical composition as defined herein.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviours, and atypical variations of structure and function, while in other contexts and for other purposes these may be considered distinguishable categories.

The term “infectious disease” refers to any disease which can be transmitted from individual to individual or from organism to organism, and is caused by a microbial agent. Infectious diseases are known in the art and include, for example, a viral disease, a bacterial disease, or a parasitic disease, which diseases are caused by a virus, a bacterium, and a parasite, respectively. In this regard, the infectious disease can be, for example, sexually transmitted diseases (e.g., chlamydia, gonorrhoea, or syphilis), SARS, coronavirus diseases (e.g., COVID-19), acquired immune deficiency syndrome (AIDS), measles, chicken pox, cytomegalovirus infections, herpes simplex virus (e.g., HSV-1, HSV-2), hepatitis (such as hepatitis B or C), influenza (flu, such as human flu, swine flu, dog flu, horse flu, and avian flu), HPV infection, shingles, rabies, common cold, gastroenteritis, rubella, mumps, anthrax, cholera, diphtheria, foodborne illnesses, leprosy, meningitis, peptic ulcer disease, pneumonia, sepsis, septic shock, tetanus, tuberculosis, typhoid fever, urinary tract infection, Lyme disease, Rocky Mountain spotted fever, chlamydia, pertussis, tetanus, meningitis, scarlet fever, malaria, trypanosomiasis, Chagas disease, leishmaniasis, trichomoniasis, dientamoebiasis, giardiasis, amoebic dysentery, coccidiosis, toxoplasmosis, sarcocystosis, rhinosporidiosis, and balantidiasis.

In some embodiments, the nucleic acid-lipid particle or a pharmaceutical composition described herein may be used in the therapeutic or prophylactic treatment of an infectious disease.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In some embodiments of the disclosure, the aim is to provide protection against an infectious disease by vaccination.

In some embodiments of the disclosure, the aim is to provide secreted therapeutic proteins, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments, the compositions described herein, which contain mRNA encoding a SARS-CoV-2 S protein, an immunogenic variant thereof, or an immunogenic fragment of the SARS-CoV-2 S protein or the immunogenic variant thereof (in the following simply “SARS-CoV-2 S nucleic acid compositions” which explicitly include SARS-CoV-2 S RNA compositions), following administration to a subject, induce an antibody response, in particular a neutralizing antibody response, in the subject that targets a panel of different S protein variants such as SARS-CoV-2 S protein variants, in particular naturally occurring S protein variants. In some embodiments, the panel of different S protein variants comprises at least 5, at least 10, at least 15, or even more S protein variants. In some embodiments, such S protein variants comprise variants having amino acid modifications in the RBD domain and/or variants having amino acid modifications outside the RBD domain. In some embodiments, the SARS-CoV-2 S nucleic acid compositions described herein following administration to a subject induce an immune response (cellular and/or antibody response, in particular a neutralizing antibody response) in the subject that targets VOC-202012/01.

In some embodiments, the SARS-CoV-2 S nucleic acid compositions described herein following administration to a subject induce an immune response (cellular and/or antibody response, in particular a neutralizing antibody response) in the subject that targets 501.V2.

In some embodiments, the SARS-CoV-2 S nucleic acid compositions described herein following administration to a subject induce an immune response (cellular and/or antibody response, in particular a neutralizing antibody response) in the subject that targets “Cluster 5”.

In some embodiments, the SARS-CoV-2 S nucleic acid compositions described herein following administration to a subject induce an immune response (cellular and/or antibody response, in particular a neutralizing antibody response) in the subject that targets “B.1.1.28”.

In some embodiments, the SARS-CoV-2 S nucleic acid compositions described herein following administration to a subject induce an immune response (cellular and/or antibody response, in particular a neutralizing antibody response) in the subject that targets “B.1.1.248”.

In some embodiments, the SARS-CoV-2 S nucleic acid compositions described herein following administration to a subject induce an immune response (cellular and/or antibody response, in particular a neutralizing antibody response) in the subject that targets the Omicron (B.1.1.529) variant.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

The terms “immunization” or “vaccination” describe the process of administering an antigen to an individual with the purpose of inducing an immune response, for example, for therapeutic or prophylactic reasons.

### EXAMPLES

The Examples provide detailed information about the manufacturing process according to the invention, highlighting the problems that are overcome by the current invention using a model formulation comprising of ionizable lipid (HY501), DSPC (1,2-distearoyl-sn-glycero-3-phosphocholine) and cholesterol as helper lipids, and C14 amine-terminated polysarcosine (NH-Psar)23 as a stealth moiety. The final drug product formulated in this invention uses luciferase as the RNA payload.

### Example 1: Formulations Containing Grafted Lipid

The manufacturing was performed by the two-step process: (i) manufacturing of pre-formed lipid nanoparticles (i.e. the aqueous dispersion) and (ii) mixing of the pre-formed lipid nanoparticles with mRNA. The detail of each step is further described below.

### (i) Manufacturing of HY501/C14PSar(23)-NH Pre-Formed Lipid Nanoparticles by Solvent Injection

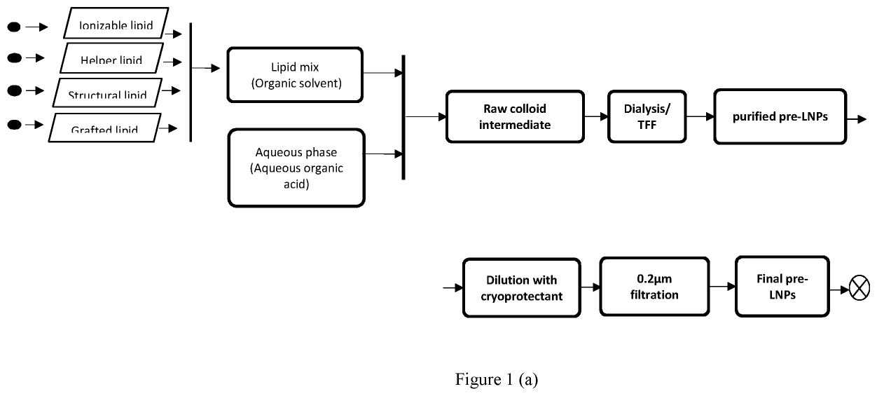

Pre-formed lipid nanoparticles were manufactured by a fluid path mixing of an organic phase containing dissolved lipids with an aqueous phase (5 mM acetic acid (AcOH), pH about 3.5). The mixing was achieved using syringe pumps and T-piece as a mixing element at a total flow rate of 200 mL/min and a volume ratio of 1:4 (organic:aqueous). The lipid mixture (50.0 mM total concentration) was composed of a cationically ionizable lipid HY-501, cholesterol, DSPC, and C14-Psar(23)-NH at a molar ratio of 47.5:38.5:10:4 dissolved in isopropanol respectively. The organic solvent in the obtained raw colloid nanoparticles was removed by diafiltration against 5 mM AcOH using tangential flow filtration (TFF) with hollow fiber (mPES/100 kD, REPLIGEN) and was concentrated if required. After TFF, the nanoparticles were diluted with 40% sucrose in 5 mM AcOH to a sucrose concentration of 10%, filtered through a 0.22 μm polyethersulfone (PES) filter, and stored at −20° C. until further used for RNA-lipid particle manufacturing. A schematic representation of the manufacturing of preformed lipid nanoparticles is reprinted in FIG. 1.

(ii) Mixing of Pre-Formed Lipid Nanoparticles with mRNA

RNA-lipid particles were prepared by complexing an aqueous dispersion of pre-formed lipid nanoparticles with N1-methylpseudouridine-modified mRNA encoding a model antigen (modRNA). This was done by mixing equal volumes of RNA and pre-formed lipid nanoparticle phases in a T-shaped mixing channel (inner diameter of 2.4 mm) at a total flow rate of 360 mL/min using a semi-automated process.

The RNA phase was prepared by dilution of the modRNA in its native buffer, i.e., 10 mM HEPES/0.1 mM EDTA at pH 7, to an RNA concentration of 0.25 mg/mL. The second phase contained an aqueous dispersion of pre-formed lipid nanoparticles containing HY501, Cholesterol, DSPC, and NH-Psar in molar ratios of 47.5:38.5:10:4 respectively. The pre-formed lipid nanoparticles were provided in 5 mM acetic acid and 10% (w/v) sucrose at pH 4.5. If required, the pre-formed lipid nanoparticles were further diluted to a target concentration with its native buffer matrix. The two phases were mixed at an N/P ratio of 6 and a total flow rate of 360 mL/min.

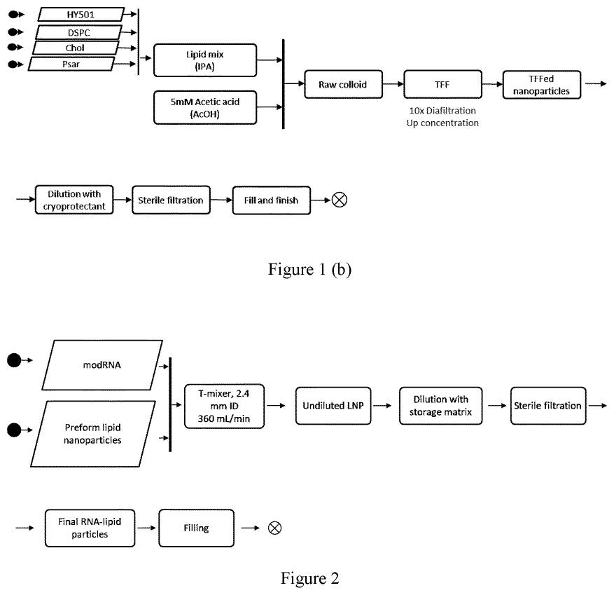

After mixing the two streams of RNA and pre-formed lipid particles, the raw RNA-lipid nanoparticles, with an RNA concentration of 0.125 mg/mL were conditioned by dilution with a storage matrix comprising 60 mM HEPES, 30% (w/v) sucrose to a final RNA concentration of 0.1 mg/mL and target pH of approximately 5.5. In the next step, the RNA-lipid particles were sterile-filtered using a 0.22 m polyethersulfone (PES) filter and filled into vials. All manufacturing processes were performed at room temperature. The composition of the formulation is presented in Table 1. A schematic representation of the RNA-lipid particle manufacturing process is also depicted in FIG. 2.

TABLE 1
Composition of exemplary RNA-lipid
particles used in this invention

| Component | Concentration | |
|---|---|---|
| RNA | 0.10 | mg/mL |
| HEPES | 3.81 | mg/mL |
| EDTA | 0.01 | mg/mL |
| HY501 | 1.38 | mg/mL |
| NH-Psar | 0.28 | mg/mL |
| DSPC | 0.30 | mg/mL |
| Cholesterol | 0.57 | mg/mL |
| Acetic acid | 0.12 | mg/mL |
| Sucrose | 100.00 | mg/mL |

### Stability of Pre-Formed Lipid Nanoparticles

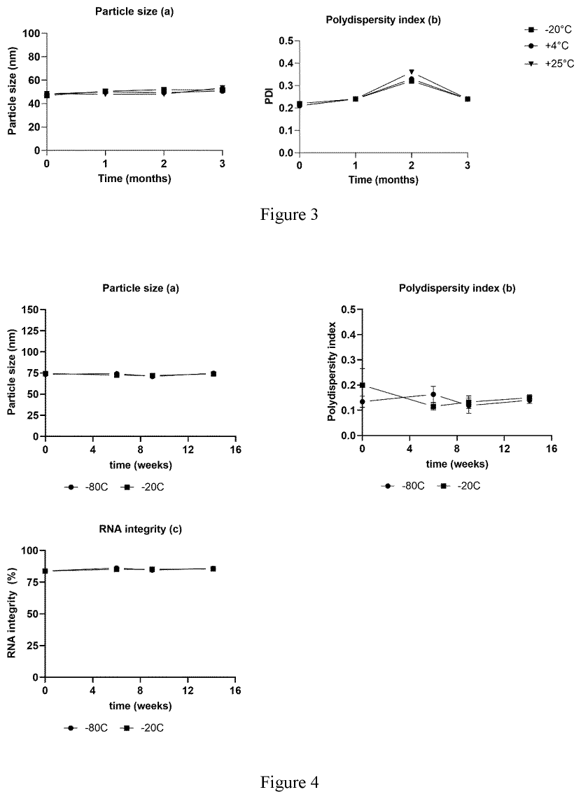

The stability of the pre-formed lipid nanoparticles was investigated. It was observed that the colloidal stability of nanoparticles at both liquid (2-8° C. and 25° C.) and frozen (−20° C.) conditions were maintained for the currently evaluated 3 months, as depicted in FIG. 3.

### Long-Term Stability of RNA-Lipid Particles

The long-term stability of the RNA-lipid particles was investigated in a stability study. As observed in FIG. 4, no changes in particle size, polydispersity or RNA integrity were observed as a function of time when the drug product is stored frozen at −20° C. or at −80° C.

The biological functionality of the RNA-lipid particles produced by the method of the invention (LNP 2) was compared to RNA-lipid particles produced by the conventional LNP manufacturing process (LNP 1). Here the two drug products were compared in an in vivo setting by intramuscular route of administration using a model antigen. The administration regime was prime administration at Day 1 followed by a booster after 21 days.

As shown in FIG. 5, all groups tested showed higher CD8+ peptide pool than CD4+ peptide pool. The RNA-lipid particles produced by the two-step process of the invention (LNP 2) showed higher T-cell response compared to the particles produced by the traditional LNP manufacturing process (one-step process) (LNP 1).

### Example 2: Manufacturing of DODMA/C14-Psar(23)-Ac Pre-Formed Lipid Nanoparticles by Ethanol Injection

The stability of the pre-formed lipid nanoparticles was investigated. Pre-formed lipid nanoparticles were manufactured by a fluid path mixing of an organic phase containing dissolved lipids with an aqueous phase (5 mM AcOH, pH 3.5). The mixing was achieved using syringe pumps and T-piece as a mixing element at a total flow rate of 200 mL/min and a volume ratio of 1:4 (organic:aqueous). The lipid mixture was composed of a cationically ionizable lipid (DODMA), cholesterol, DSPC, and C14-PSar(23)-Ac dissolved in ethanol at a molar ratio of 47.5:38.5:10:4 respectively. The organic solvent in the obtained raw colloid nanoparticles was removed by diafiltration against 5 mM AcOH using tangential flow filtration (TFF) with hollow fiber (mPES/100 kD, REPLIGEN) and was concentrated to two times (2×). After TFF, the nanoparticles were diluted to a sucrose concentration of 10% if required, filtered through a 0.22 μm polyethersulfone (PES) filter and stored at 4° C. until further used for RNA-lipid particle manufacturing.

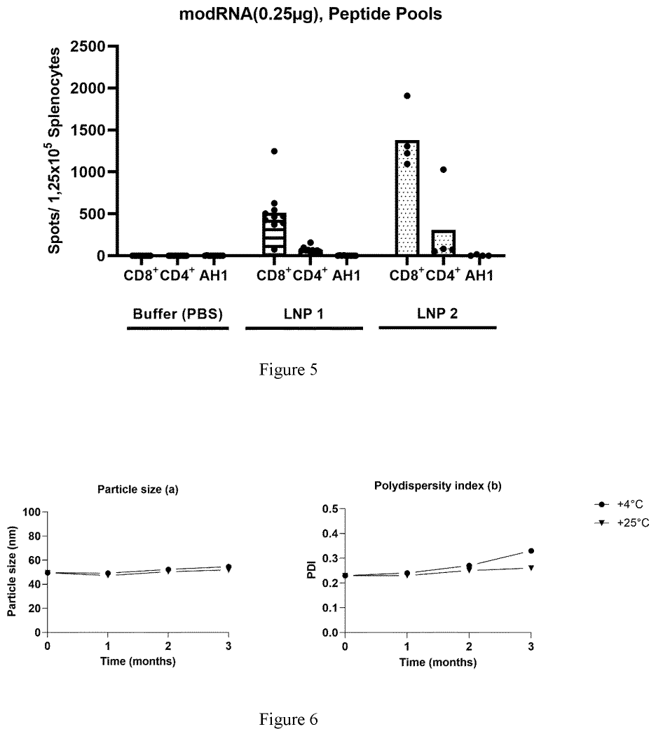

The stability of the pre-formed lipid nanoparticles was investigated, and it was observed that the colloidal stability of nanoparticles at 2-8° C. and 25° C. conditions were maintained for the currently evaluated 3 months as depicted in FIG. 6.

### Example 3: Manufacturing of DODMA/DMG-PEG2k Pre-Formed Lipid Nanoparticles by Ethanol Injection

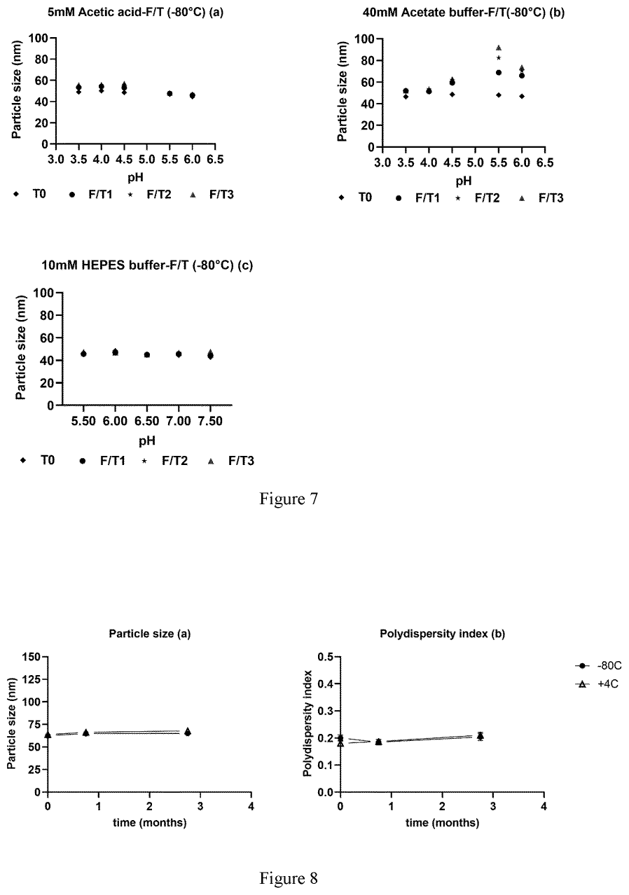

The effect of different buffer conditions on the stability of pre-formed lipid nanoparticles was investigated. For preparing pre-formed lipid nanoparticles, a lipid mix composed of a cationically ionizable lipid (DODMA), cholesterol, DSPC, and DMG-PEG2k dissolved in ethanol at a molar ratio of 47.5:40.7:10:1.8 respectively and mixed with an aqueous phase (5 mM AcOH) using a standard syringe pump based set-up and a T-piece as a mixing element at a total flow rate of 200 mL/min and a volume ratio of 1:4 (organic:aqueous). The organic solvent in the obtained raw colloid nanoparticles was removed by dialysis against either (i) 5 mM AcOH, (ii) 40 mM acetate buffer, or (iii) 10 mM HEPES, across a range of different pHs (see FIG. 7), in Slide-A-Lyzer dialysis cassettes of 10K molecular weight cut-off (MWCO) (Thermo Fisher Scientific, Waltham, MA, USA). After dialysis, the nanoparticles were diluted to a sucrose concentration of 10%, filtered through a 0.22 μm polyethersulfone (PES) filter, and freeze-thaw and stability studies were performed at different conditions.

Freeze-thaw studies were conducted by cycling the nanoparticles from −80° C. (overnight) to room temperature (25° C.) (2 h) for at least three times. The nanoparticles between the thaw and freeze cycles were mixed by gentle inversions before the next freezing cycle. The particle size and polydispersity index of the nanoparticles were measured after each freeze-thaw cycle.

### Stability of DODMA/DMG-PEG2k Pre-Formed Lipid Nanoparticles

It was observed that the colloidal stability of nanoparticles was maintained with 5 mM acetic acid and 10 mM HEPES at different pHs over all three freeze-thaw cycles but in the case of 40 mM acetate buffer, particles size increased over all the freeze-thaw cycles as depicted in FIG. 7).

### Example 4: Manufacturing of HY501/10% DSPC (Grafted-Lipid Free) RNA-Lipid Particles

Pre-formed lipid nanoparticles were manufactured by a fluid path mixing of an organic phase containing dissolved lipids with an aqueous phase (5 mM AcOH). The mixing was achieved using syringe pumps and T-piece as a mixing element at a total flow rate of 200 mL/min). The lipid mixture (50.0 mM total concentration) was composed of an ionizable lipid HY-501, cholesterol, and DSPC at a molar ratio of 47.5:42.5:10 dissolved in isopropanol respectively. The organic solvent in the obtained raw colloid nanoparticles was removed by diafiltration against 5 mM AcOH using tangential flow filtration (TFF) with hollow fiber (mPES/100 kD, REPLIGEN) and was concentrated 2× times. After TFF, the nanoparticles were diluted to a sucrose concentration of 10%, filtered through a 0.22 μm polyethersulfone (PES) filter, and stored at −20° C. until further used for RNA-lipid particle manufacturing. The particle size of the pre-formed lipid nanoparticles was ~40 nm with polydispersity index of ~0.2.

The pre-formed lipid nanoparticles were mixed with RNA phase as described herein, and the stability of the resulting RNA-lipid nanoparticles was analyzed. As shown in FIG. 8, the colloidal properties of the formulation could be maintained in both frozen and liquid conditions over a period of at least 3 months.

### Example 5: Manufacturing of HY501/C14-pSar(23)-NH Pre-Formed Lipid Nanoparticles with Malic Acid by Solvent Injection

Pre-formed lipid nanoparticles were manufactured by a fluid path mixing of an organic phase containing dissolved lipids with an aqueous phase (2.5 mM malic acid, about pH 2.6). The mixing was achieved using syringe pumps and T-piece as a mixing element at a total flow rate of 200 mL/min and a volume ratio of 1:4 (organic:aqueous). The lipid mixture (50.0 mM total concentration) was composed of a cationically ionizable lipid HY-501, cholesterol, DSPC, and C14-pSar(23)-NH at a molar ratio of 47.5:38.5:10:4 dissolved in isopropanol respectively. The organic solvent in the obtained raw colloid nanoparticles was removed by diafiltration against 2.5 mM Malic acid using tangential flow filtration (TFF) with hollow fiber (mPES/100 kD, REPLIGEN) and was concentrated if required. After TFF, the nanoparticles were diluted with a solution containing 40% sucrose, 2.5 mM malic acid to a sucrose concentration of 10%, filtered through a 0.22 μm polyethersulfone (PES) filter, and stored at −20° C. until further used for RNA-lipid particle manufacturing.

Preparation and characterization of functionalized RNA-lipid particles: Alfa tagged RNA-lipid particles can be prepared using a known aqueous/organic manufacturing protocol such as described in WO 2013/143683 or WO 2020/201383.

### Example 6: Manufacturing of Pre-Formed Lipid Nanoparticles (DODMA/C14-pSar(23)-Ac/Alfa-Tag Lipid) by Ethanol Injection

For preparing alfa-tagged pre-formed lipid nanoparticles, a lipid mix consisting of DODMA, DSPC, cholesterol, C14-pSar(23)-Ac and DSPE-PEG2k-alfa peptide in a molar ratio of 47.5:10:38.3:4:0.2 was dissolved in organic solvent (ethanol) at 50 mM total lipid concentration and mixed with an aqueous phase (5 mM AcOH) using a standard syringe pump based set-up and a T-piece as a mixing element at a total flow rate of 200 mL/min. The organic solvent in the obtained raw colloid nanoparticles was removed by diafiltration against 5 mM AcOH using tangential flow filtration (TFF) with hollow fiber (mPES/100 kD, REPLIGEN) and was concentrated to 2× times if required. After TFF, the nanoparticles were diluted to a sucrose concentration of 10% and filtered through a 0.22 μm polyethersulfone (PES) filter.

### Stability of Pre-Formed Lipid (DODMA PSar-Ac Alfa-Tag Lipid) Nanoparticles

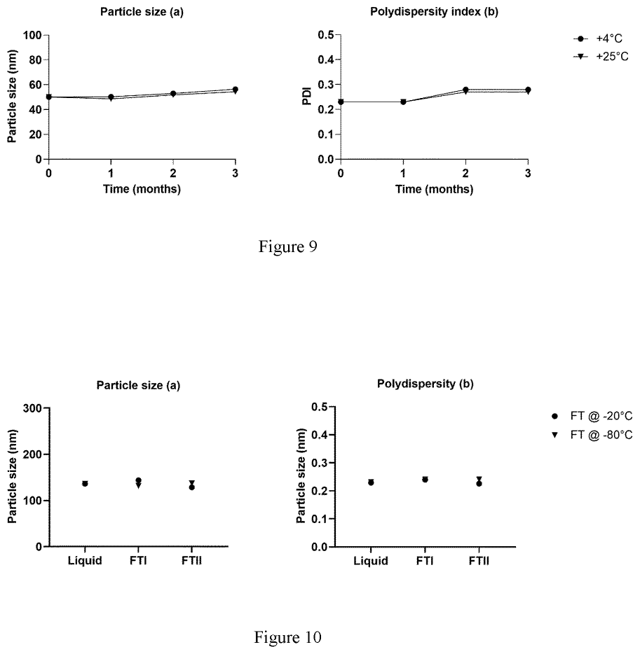

The stability of the pre-formed lipid nanoparticles has been investigated. It was observed that the colloidal stability of nanoparticles at 2-8° C. and 25° C. conditions were maintained for the currently evaluated 3 months (stability ongoing) as depicted in FIG. 9.

### Manufacturing of Functionalized RNA-Lipid Particles by Aqueous-Aqueous Protocol

RNA-lipid particles were prepared by an aqueous-aqueous protocol, as described herein. Briefly, RNA in aqueous buffer conditions of HEPES 10 mM, EDTA 0.1 mM, pH 7.0 was mixed with pre-formed lipid nanoparticles composed of DODMA, DSPC, cholesterol, C14-pSar(23)-Ac and DSPE-PEG2k-alfa peptide in molar ratio of 47.5:10:38.3:4:0.2, respectively, in aqueous solution of 5 mM acetic acid, 10% sucrose, in a volume ratio of 1:1. The mixing was achieved using a standard syringe pump based set-up with a total flow of 360 ml/ml (180 ml/min for each phase) using T mixing element (2.4 mm T mixer, 1.6 mm tubing). The resulting RNA-lipid particles were then functionalized with an aCD3-VHH ligand and further diluted with respective buffer of choice supplemented with sucrose to a final RNA concentration of 0.1 mg/mL and target pH of approximately 5.5. Typically, the RNA-lipid particles were prepared at N/P ratio of 6:1.

### Freeze-Thaw Studies

Freeze-thaw studies were conducted by cycling the formulations from −20° C. and −80° C. (overnight) to +25° C. (2 h) for at least two times. The particle size and polydispersity index of the formulations were measured for freeze-thaw samples. The formulations between thaw and freeze cycles were mixed by gentle inversions before the next freezing cycle.

Size and polydispersity index (PDI) were determined for (i) alfa-tagged pre-formed lipid nanoparticles before RNA-lipid particle manufacturing, (ii) alfa-tagged RNA-lipid particles immediately after manufacturing (raw alfa-RNA-lipid particles), and (iii) functionalized RNA-lipid particles. Functionalization was performed in a 13:1 v/v raw alfa-RNA-lipid particles:aCD3-VHH ratio, at ligand/cargo ratio of 0.48 w/w. It was observed that alfa-tagged RNA-lipid particles can be manufactured with microfluidics starting from alfa-tagged pre-formed lipid nanoparticles stabilized in the presence of sucrose, with controlled particles sizes. Moreover, alfa-tagged RNA-lipid particles can be successfully functionalized with aCD3-VHH protein, resulting in particles of around −137 nm in size.

FIG. 10 shows the results from freezing of a CD3-functionalized RNA-lipid particles at −20° C. and −80° C., respectively, and storage for at least 2 freeze/thaw cycles. As can be seen, no significant changes in size and PDI could be observed at both freezing temperatures tested.

### Example 7: Preparation and Characterization of Functionalized RNA-Lipid Particles with Alfa-Lipid Post Insertion

### Manufacturing of Pre-Formed Lipid Nanoparticles (HY501/10% DSPC) by Ethanol Injection

Manufacturing of HY501/10% DSPC (grafted-lipid free) pre-formed lipid nanoparticles by ethanol injection was performed as described above in Example 4.

### Manufacturing of Functionalized RNA-Lipid Particles by Aqueous-Aqueous Protocol

RNA-lipid particles were prepared by an aqueous-aqueous protocol, as described herein. Briefly, RNA in aqueous buffer conditions of HEPES 10 mM, EDTA 0.1 mM, pH 7.0 was mixed with pre-formed lipid nanoparticles composed of HY501, DSPC, cholesterol in molar ratio of 47.5:10:42.5, respectively in aqueous solution of 5 mM acetic acid, 10% sucrose, in a volume ratio of 1:1. The mixing was achieved using a standard syringe pump based set-up with a total flow of 360 ml/min (180 ml/min for each phase) using T mixing element (2.4 mm T mixer, 1.6 mm tubing). The resulting RNA-lipid particle stock was then mixed with a stock solution of DSPE-PEG2k-alfa peptide to have 0.2 final molar ratio. The RNA-lipid particle stock was then functionalized with the aCD3-VHH ligand and further diluted with a storage matrix comprising 60 mM HEPES, 30% (w/v) sucrose to a final RNA concentration of 0.1 mg/mL and target pH of approximately 5.5. In the benchmark settings, the RNA-lipid particles are prepared at N/P ratio of 6:1.

### Freeze-Thaw Studies

Freeze-thaw studies were conducted by cycling the formulations from −80° C. (overnight) to ±25° C. (2 h) for at least two times. The particles size and polydispersity index of the formulations was measured for freeze-thaw samples.

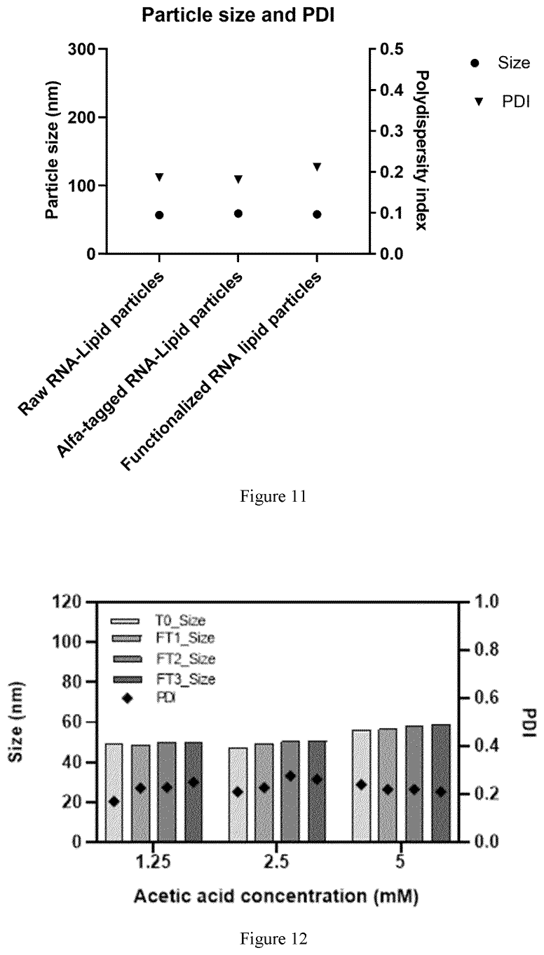

Size and PDI was determined for (i) pre-formed lipid nanoparticles before RNA-lipid particle manufacturing, (ii) RNA-lipid particles immediately after manufacturing (raw RNA-lipid particles), (iii) RNA-lipid particles after DSPE-PEG2k-alfa peptide post insertion (Alfa-RNA-lipid particles), and (iv) functionalized RNA-lipid particles. Functionalization was performed in a 27:1 v/v raw alfa-RNA-lipid particles:aCD3-VHH ratio, at ligand/cargo ratio of 0.48 w/w. It was observed that alfa-tagged RNA-lipid particles can be manufactured with controlled particles sizes using post-insertion of alfa-lipid starting from pre-formed lipid nanoparticles stabilized in the presence of sucrose. Moreover, alfa-tagged RNA-lipid particles can be successfully functionalized with aCD3-VHH protein, resulting in particles of around −60 nm in size (FIG. 11).

### Example 8: Pre-Formed Lipid Nanoparticles Preparation by Thin Film Hydration Method

Pre-formed lipid nanoparticles were manufactured using the film hydration method. The lipid mixture (50.0 mM total lipid concentration) was composed of cationic lipid (DODMA), cholesterol, DSPC, and DMG-PEG dissolved in chloroform or dichloromethane:ethanol (1:1) mix at molar ratio of 47.5:40.7:10:1.8 respectively and was poured into a round bottom flask. With rotary evaporation, the organic solvent was evaporated leaving a thin lipid film on the inner walls of the flask and the lipid film was kept under a high vacuum (1-2 hours) for drying. The thin film was then hydrated by adding the required quantity of aqueous phase (organic acid, e.g., acetic acid, or malic acid) and allowed to rotate in the rotary water bath at room temperature for 1 hour. Next, hydrated aqueous lipid dispersion (raw colloid) size was reduced using sequential extrusion through 200 nm and 100 nm pore size membranes (Whatman® filter) supported by filter support for 10 times or using sonication and high-pressure homogenizer (HPH). After achieving desired particle size, the nanoparticles were diluted to a sucrose concentration of 10% if required, and filtered through a 0.22 μm polyethersulfone (PES) filter.

### Example 9: Pre-Formed Lipid Nanoparticles Preparation by Emulsification Method

A lipid mixture (50.0 mM total lipid concentration) composed of cationic lipid (DODMA), cholesterol, DSPC, and DMG-PEG was dissolved in chloroform or chloroform:ethanol (1:1) mix at molar ratios of 47.5:40.7:10:1.8 respectively and was poured into a round bottom flask. With rotary evaporation, the organic solvent was evaporated. Again, organic solvent (chloroform or ethanol) and the aqueous phase were added to the lipid mixture at a 3:1 ratio respectively and the organic phase was evaporated by rotary evaporation under reduced pressure. The acquired aqueous dispersion was sonicated in an ultrasonic bath with intermittent shaking. The pre-formed nanoparticles obtained in this way were large. Next, the aqueous dispersion size was reduced using sequential extrusion.

### Example 10: Manufacturing of ALC-0315 Pre-Formed Lipid Nanoparticles (Pre-LNP) Using Acetic Acid, and Dilution with Different Cryoprotectants

In all cases, pre-LNPs were manufactured by a fluid path mixing of an organic phase containing dissolved lipids, with an aqueous phase (as indicated). The mixing was achieved using Knauer pumps and T-piece as a mixing element at a total flow rate of 240 mL/min and a volume ratio of 1:3 (organic:aqueous). The lipid mixture (80.0 mM total concentration) composed of the cationically ionizable lipid ALC-0315, cholesterol, DSPC, and the grafted lipid ALC-0159 at a molar ratio of 47.5:40.7:10:1.8 was dissolved in ethanol respectively. The organic solvent in the obtained raw colloid nanoparticles was removed by tangential flow filtration (TFF) against the indicated solution using modified PEG hollow fiber (100K MWCO). After TFF, the nanoparticles were diluted with the indicated storage matrix and filtered through a 0.22 μm polyethersulfone (PES) filter, frozen and stored at −20° C. Freeze thaw studies were conducted by cycling the pre-LNPs from −20° C. (overnight) to room temperature (25° C.) (2 h) for at least three times. Between the thaw and freeze cycles the pre-LNPs were mixed by gentle inversions. The particle size and polydispersity index (PDI) of the pre-LNPs were measured after each freeze-thaw cycle.

10A) Dilution with 10% w/v Sucrose

Pre-LNPs were manufactured with an aqueous phase of 1.25 mM, 2.5 mM or 5 mM acetic acid (AcOH). TFF was performed against the specified concentration of acetic acid (1.25 mM, 2.5 mM and 5 mM). The pre-LNPs were then diluted to a sucrose concentration of 10% w/v. FIG. 12 shows the freeze thaw stability of the pre-LNPs manufactured with varying concentration of acetic acid and in 10% sucrose. The particle sizes and PDI remained controlled over the three freeze thaw cycles, demonstrating that different concentrations of acetic acid+10% sucrose provide very promising colloidal stability of pre-LNPs.

10B) Dilution with 8% or 12% w/v Sucrose

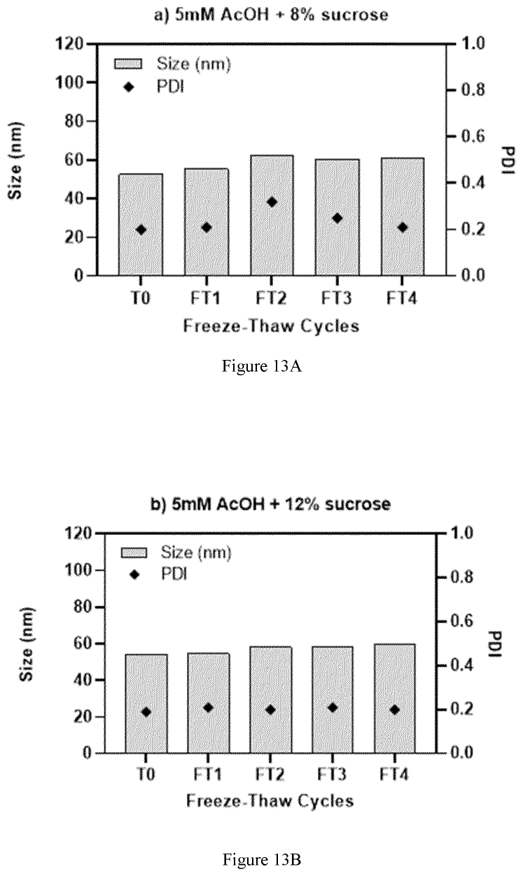

Pre-LNPs were manufactured with an aqueous phase of 5 mM AcOH. TFF was performed against 5 mM AcOH. The pre-LNPs were then diluted to a sucrose concentration of either 8% or 12% w/v. FIG. 13 shows the freeze-thaw stability of the pre-LNPs diluted with either 8% (w/v) sucrose (FIG. 13A) or 12% (w/v) sucrose (FIG. 13B). The particle sizes and PDI remained controlled at both sucrose concentrations, demonstrating that cryoprotectant concentration may be varied in the storage matrix and still provide good colloidal stability of pre-LNPs. Additionally, utilizing organic acids for the upstream and downstream processing of the pre-LNPs provided excellent colloidal stability.

10C) Dilution with 10% w/v Trehalose

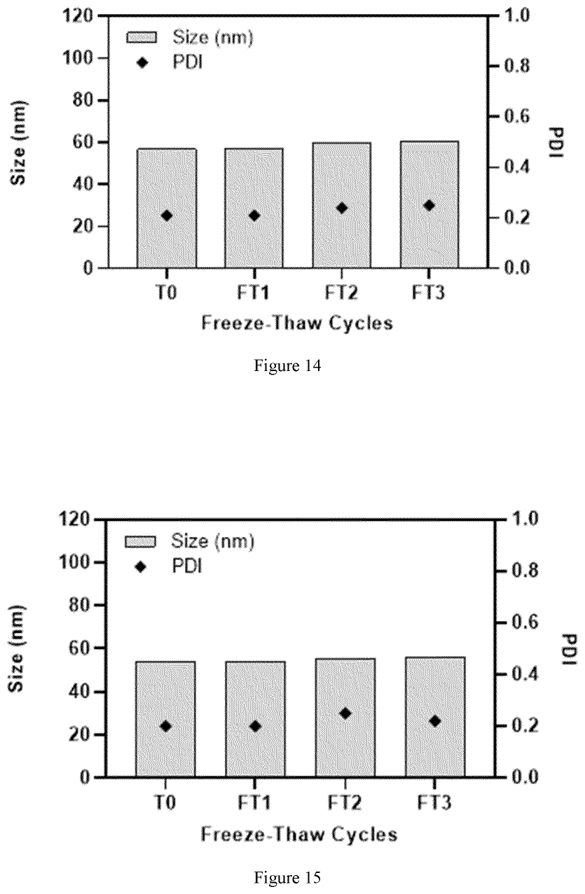

Pre-LNPs were manufactured with an aqueous phase of 5 mM AcOH. TFF was performed against 5 mM AcOH. The pre-LNPs were then diluted to a trehalose concentration of 10% (w/v). FIG. 14 shows the freeze-thaw stability of the pre-LNPs. The particle sizes and polydispersity remained controlled over the three freeze-thaw cycles, demonstrating that trehalose as cryoprotectant also provides good colloidal stability of pre-LNPs. 10D) Dilution with 5% w/v Glucose

Pre-LNPs were manufactured with an aqueous phase of 5 mM AcOH. TFF was performed against 5 mM AcOH. The pre-LNPs were then diluted to a glucose concentration of 5% w/v. FIG. 15 shows the freeze-thaw stability of the pre-LNPs. The particle sizes and PDI remained controlled over the three freeze-thaw cycles, demonstrating that glucose also provides good colloidal stability of pre-LNPs.

### Example 11: Manufacturing of BHD-C2C2-PipZ Pre-LNPs Using Acetic Acid

The pre-LNPs were manufactured by a fluid path mixing of an organic phase containing dissolved lipids, with an aqueous phase either (i) 2.5 mM AcOH, or (ii) 5 mM AcOH, both about pH 3.5. The mixing was achieved using syringe pumps and T-piece as a mixing element at a total flow rate of 90 mL/min and a volume ratio of 1:3 (organic:aqueous). The lipid mixture (80.0 mM total concentration) was composed of a cationically ionizable lipid BHD-C2C2-PipZ, cholesterol, DSPC, and α-tocopherol-pAEEA14 at a molar ratio of (i) 47.5:40.7:10:1.8 for 2.5 mM AcOH, or (ii) 47.5:38.5:10:4 for 5 mM AcOH, dissolved in ethanol respectively. The organic solvent in the obtained raw colloid nanoparticles was removed by dialysis in both cases against 2.5 mM AcOH, about pH 3.5 in Slide-A-Lyzer dialysis cassettes of 10K MWCO (Thermo Fisher Scientific, Waltham, MA, USA). After dialysis, the nanoparticles were diluted with sucrose and filtered through a 0.22 μm polyethersulfone (PES) filter.

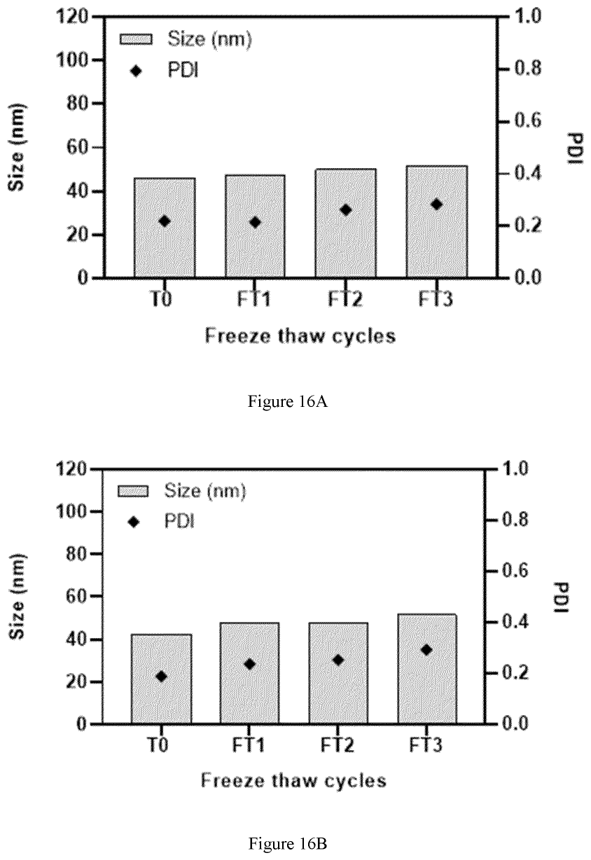

Freeze-thaw studies were conducted by cycling the pre-LNPs from −20° C. (overnight) to room temperature (25° C.) (2 h) for at least three times. Between the thaw and freeze cycles the pre-LNPs were mixed by gentle inversions. The particle size and PDI of the nanoparticles were measured after each freeze thaw cycle. FIG. 16 shows the freeze thaw stability of the pre-LNPs manufactured with 2.5 mM AcOH (FIG. 16A) or 5 mM AcOH (FIG. 16B). The particle sizes and polydispersity remained controlled within acceptable limits over the three freeze-thaw cycles, demonstrating the promising colloidal stability of pre-LNPs containing BHD-C2C2-PipZ manufacturing in different concentrations of AcOH.

### Example 12: Manufacturing of ALC-0315 Pre-LNPs Using Malic Acid

The pre-LNPs were manufactured by a fluid path mixing of an organic phase containing dissolved lipids, with an aqueous phase (as indicated). The mixing was achieved using syringe pumps and T-piece as a mixing element at a total flow rate of 90 mL/min and a volume ratio of 1:3 (organic:aqueous). The lipid mixture (80.0 mM total concentration) was composed of ALC-0315, cholesterol, DSPC, and ALC-0159 at a molar ratio of 47.5:40.7:10:1.8 dissolved in ethanol respectively. The organic solvent in the obtained raw colloid nanoparticles was removed by dialysis against the indicated solution, in Slide-A-Lyzer dialysis cassettes of 10K MWCO (Thermo Fisher Scientific, Waltham, MA, USA). After dialysis, the nanoparticles were diluted to a sucrose concentration of 10% and filtered through a 0.22 μm polyethersulfone (PES) filter.

Freeze-thaw studies were conducted by cycling the nanoparticles from −20° C. (overnight) to room temperature (25° C.) (2 h) for at least three times. Between the thaw and freeze cycles the pre-LNPs were mixed by gentle inversions. The particle size and PDI of the nanoparticles were measured after each freeze-thaw cycle.

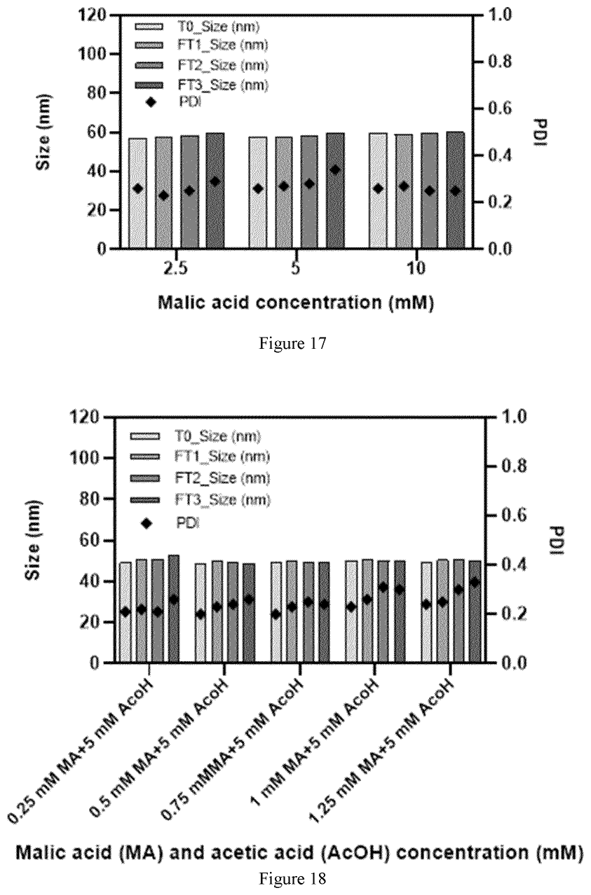

FIG. 17 shows the results of freeze-thaw studies for pre-LNPs manufactured using as aqueous phase malic acid at varying concentrations: (i) 2.5 mM, (ii) 5 mM, or 10 mM malic acid, (about pH 3-4); where dialysis was performed, respectively, also against (i) 2.5 mM, (ii) 5 mM, or 10 mM malic acid, (about pH 3-4). The particle sizes and PDI remained controlled over the three freeze thaw cycles (FIG. 7), demonstrating the very promising colloidal stability of pre-LNPs manufactured with malic acid.

FIG. 18 shows the results of freeze-thaw studies for pre-LNPs manufactured using as aqueous phase a mixture of acetic acid (5 mM) plus malic acid at varying concentrations (over 0.25 mM to 1.25 mM), about pH 3-4; where dialysis was performed, respectively, also against (i) 5 mM acetic acid, 0.25 mM malic acid; (ii) 5 mM acetic acid, 0.5 mM malic acid; (iii) 5 mM acetic acid, 0.75 mM malic acid; (iv) 5 mM acetic acid, 1.0 mM malic acid; and (v) 5 mM acetic acid, 1.25 mM malic acid, (all about pH 3-4). The particle sizes and PDI remained controlled over the three freeze-thaw cycles (FIG. 8), demonstrating the very promising colloidal stability of pre-LNPs manufactured with mixtures of malic acid and acetic acid.

### Example 13: Lyophilized Acetic Acid-Based Pre-LNPs

The pre-LNPs were manufactured by a fluid path mixing of an organic phase containing dissolved lipids, with an aqueous phase (5 mM acetic acid). The mixing was achieved using Knauer pumps and T-piece as a mixing element at a total flow rate of 240 mL/min and a volume ratio of 1:3 (organic:aqueous). The lipid mixture (80.0 mM total concentration) composed of ALC-0315, cholesterol, DSPC, and ALC-0159 at a molar ratio of 47.5:40.7:10:1.8 dissolved in ethanol respectively. The organic solvent in the obtained raw colloid nanoparticles was removed by tangential flow filtration (TFF) against respective varying concentrations of acetic acid (1.25 mM, 2.5 mM and 5 mM) using modified PEG hollow fiber (100K MWCO). After TFF, the nanoparticles were diluted to a sucrose concentration of 10% and filtered through a 0.22 μm polyethersulfone (PES) filter and lyophilized (see, e.g., WO 2022/101486 for suitable lyophilization/freeze drying protocols).

The physicochemical properties of the lyophilized pre-LNPs were then analysed and the results are shown in Table 2. The pre-LNPs showed good particle attributes after reconstitution. The particle sizes and PDI remained controlled after reconstitution, demonstrating the promising colloidal stability of pre-LNPs and the potential of lyophilization for pre-LNP storage.

TABLE 2
Physicochemical properties of pre-
LNPs before and after lyophilization.

| | Before | After |
|---|---|---|
| Parameters | lyophilization | lyophilization |
| Appearance | White to off- | White to off- |
| | white dispersion | white dispersion |
| Particle size (nm) | 57 | 58 |
| Polydispersity | 0.22 | 0.17 |
| index (PDI) | | |
| pH | 3.80 | 3.72 |
| Osmolality | 370 | 365 |
| (mOsmol/kg) | | |

### Example 14: Formation of Pre-LNPs with Sucrose Plus 5 mM Acetic Acid

The pre-LNPs were manufactured by a fluid path mixing of an organic phase containing dissolved lipids, with an aqueous phase (5 mM acetic acid plus sucrose at varying concentration, 5 and 10% w/v). The mixing was achieved using syringe pumps and T-piece as a mixing element at a total flow rate of 90 mL/min and a volume ratio of 1:3 (organic:aqueous). The lipid mixture (80.0 mM total concentration) was composed of ALC-0315, cholesterol, DSPC, and ALC-0159 at a molar ratio of 47.5:40.7:10:1.8 dissolved in ethanol respectively. The organic solvent in the obtained raw colloid nanoparticles was removed by dialysis against (i) 5 mM acetic acid, 5% w/v sucrose, or (ii) 5 mM acetic acid, 10% w/v sucrose, in Slide-A-Lyzer dialysis cassettes of 10K MWCO (Thermo Fisher Scientific, Waltham, MA, USA). After dialysis, the nanoparticles were filtered through a 0.22 μm polyethersulfone (PES) filter.

Freeze-thaw studies were conducted by cycling the pre-LNPs from −20° C. (overnight) to room temperature (25° C.) (2 h) for at least three times. Between the thaw and freeze cycles the pre-LNPs were mixed by gentle inversions. The particle size and PDI of the pre-LNPs were measured after each freeze thaw cycle.

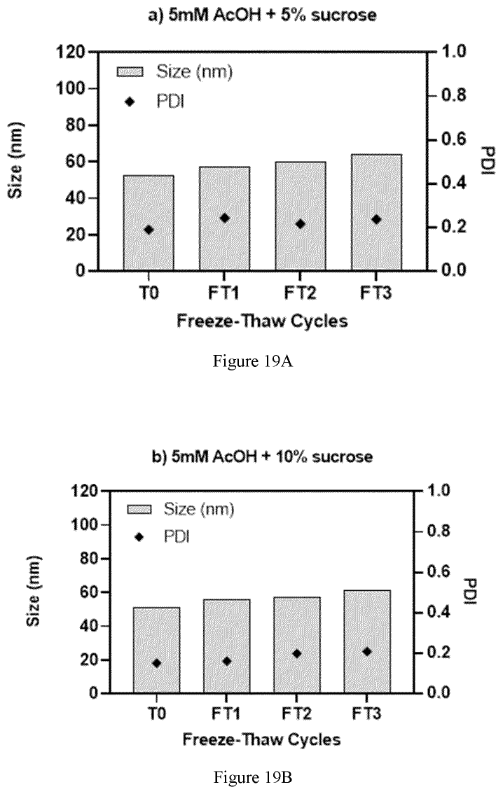

FIG. 19 shows the freeze-thaw stability of the pre-LNPs. The particle sizes and PDI remained controlled over the three freeze thaw cycles, demonstrating promising colloidal stability of pre-LNPs manufactured with sucrose present during the initial mixing step.

### Example 15 (Comparative): Formation of Pre-LNPs with Buffers Containing Inorganic Cations

In each of the following cases pre-LNPs were manufactured by a fluid path mixing of an organic phase containing dissolved lipids, with an aqueous phase (as indicated). The mixing was achieved using syringe pumps and T-piece as a mixing element at a total flow rate of 90 mL/min and a volume ratio of 1:3 (organic:aqueous). The organic solvent in the obtained raw colloid nanoparticles was removed by dialysis against the indicated solution in Slide-A-Lyzer dialysis cassettes of 10K MWCO (Thermo Fisher Scientific, Waltham, MA, USA). After dialysis, the nanoparticles were diluted to a sucrose concentration of 10% and filtered through a 0.22 μm polyethersulfone (PES) filter. Freeze thaw studies were conducted by cycling the nanoparticles from −20° C. (overnight) to room temperature (25° C.) (2 h). Between the thaw and freeze cycles the pre-LNPs were mixed by gentle inversions. The particle size and PDI of the nanoparticles were measured after each freeze thaw cycle.

### 14A) Formation of Pre-LNPs Using Acetate Buffer

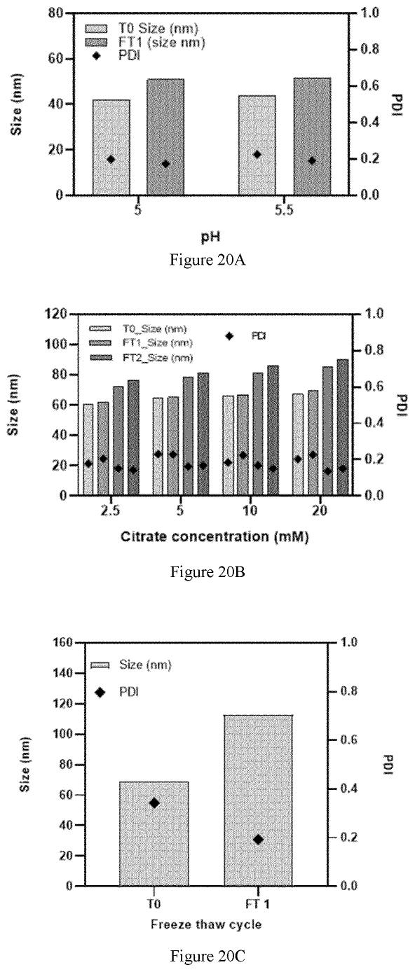

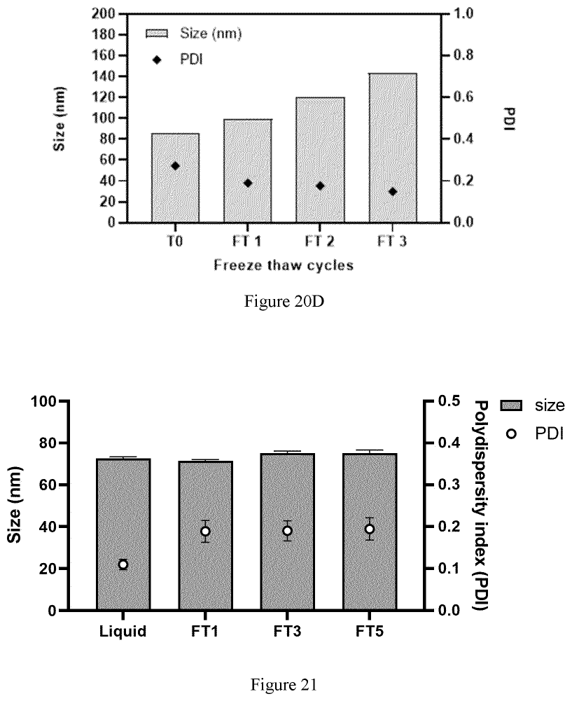

The aqueous phase used was 5 mM acetate buffer, pH 5.0 or 5.5. The lipid mixture (50.0 mM total concentration) was DODMA:cholesterol:DSPC:DMG-PEG 2000 (47.5:40.7:10:1.8 molar ratio) in ethanol. Dialysis was performed against 5 mM acetate buffer either pH 5.0 or pH 5.5. FIG. 20A shows the freeze thaw stability of the pre-LNPs. After the first freeze thaw cycle, a significant increase in the particle size was observed, therefore further studies were discontinued. This demonstrates the negative impact of buffers containing inorganic cations, such as acetate buffer, on pre-LNP colloidal stability.

### 14B) Formation of Pre-LNPs Using Citrate Buffer

The aqueous phase used was citrate buffer at 2.5 mM, 5.0 mM, 10 mM or 20 mM concentration, about pH 4. The lipid mixture (80.0 mM total concentration) was ALC-0315:cholesterol:DSPC:ALC-0159 (47.5:40.7:10:1.8 molar ratio) in ethanol. Dialysis was performed against 2.5 mM, 5.0 mM, 10 mM or 20 mM citrate buffer, as indicated, all about pH 4. FIG. 20B shows the freeze thaw stability of the pre-LNPs. After the first freeze thaw cycle, a significant and consistent increase in the particle size was observed over the freeze thaw cycles. This demonstrates the negative impact of buffers containing inorganic cations, such as citrate buffer, on pre-LNP colloidal stability.

### 14C) Formation of Pre-LNPs Using Succinate Buffer

The aqueous phase used was 30 mM succinate buffer, about pH 4. The lipid mixture (80.0 mM total concentration) was ALC-0315:cholesterol:DSPC:ALC-0159 (47.5:40.7:10:1.8 molar ratio) in ethanol. Dialysis was performed against 30 mM succinate buffer, about pH 4. FIG. 20C shows the freeze thaw stability of the pre-LNPs. After the first freeze thaw cycle, a significant increase in the particle size was observed, therefore further study was discontinued. This demonstrates the negative impact of buffers containing inorganic cations, such as succinate buffer, on pre-LNP colloidal stability.

### 14D) Formation of Pre-LNPs Using Malate Buffer

The aqueous phase used was 30 mM malate buffer about pH 4. The lipid mixture (80.0 mM total concentration) was ALC-0315:cholesterol:DSPC:ALC-0159 (47.5:40.7:10:1.8 molar ratio) in ethanol. Dialysis was performed against 30 mM malate buffer about pH 4. FIG. 20D shows the freeze thaw stability of the pre-LNPs. After each freeze thaw cycle, a consistent increase in particle size was observed over the FT cycles and the dispersion appeared more turbid. This demonstrates the negative impact of buffers containing inorganic cations, such as malate buffer, on pre-LNP colloidal stability.

### Example 16: Process of RNA-LNP Manufacturing from preLNPs

In all of the following Examples, formation of RNA-LNPs follows an exemplary manufacturing scheme as shown in FIG. 2, and unless otherwise stated has the following parameters. RNA-LNPs were prepared by complexing an aqueous dispersion of pre-LNPs (the pre-LNP phase) with an aqueous RNA phase. Pre-LNPs were composed of ALC-0315:cholesterol:DSPC:ALC-0159 lipids in a molar ratio 47.5:40.7:10:1.8, and at a concentration of ALC-0315 of 5 mM in the pre-LNP phase. The RNA phase was provided in 10 mM HEPES, 0.1 mM EDTA, pH 7 with an RNA concentration of 0.25 mg/mL. The two phases were mixed in a flow rate ratio of 1:1 using a T-shaped mixing channel at a total flow rate of 360 mL/min using a semi-automated process. The raw RNA-LNPs obtained directly after mixing had a lipid-to-RNA ratio of 6.6 and an RNA concentration of 0.125 mg/mL. The raw RNA-LNPs were further processed by dilution with a storage matrix composed of 60 mM HEPES, 3 mM Tris, 30% sucrose (w/v) at pH 6.3 to target pH of the final drug product of pH 5.3 and to target the RNA concentration of 0.1 mg/mL in final formulation. The formulation was further sterile-filtered using a 0.22 μm polyethersulfone (PES) filter, and filled into the primary packaging materials vials. All manufacturing processes were performed at room temperature. Where indicated, freeze-thaw (FT) cycling was used as a stressed condition to evaluate the potential frozen stability of the LNPs. The LNPs were cycled between −20° C. or −80° C. (as indicated)(overnight) to room temperature (25° C.) (2 h) for the indicated number of times (e.g., three freeze-thaw cycles=FT3). Particle size and polydispersity index (PDI) were monitored.

### Example 17: RNA-LNPs from Pre-LNPs in 5 mM Acetic Acid, 10% Sucrose

The pre-LNP phase was in 5 mM acetic acid in 10% sucrose (w/v) at pH 4.5 at a final concentration of ALC-0315 of 5 mM. A mixture of two RNAs in 1:1 w/w ratio was used. Colloidal stability of the manufactured RNA-LNPs was assessed after 5 freeze-thaw cycles between −20° C. and room temperature. No aggregation of the RNA-LNPs was observed after 5 freeze-thaw cycles.

Table 3: Size, polydispersity index (PDI), pH, osmolality (Osmo), sub visible particle count (SVP), encapsulation efficiency (EE) of the RNA-LNPs.

Encapsulation efficiency (EE) of LNPs was evaluated using the RiboGreen® assay. Briefly, samples of the RNA-LNPs are taken and either treated with Triton X-100 or not, and the RNA-binding fluorescent dye RiboGreen® is added. Determination of the RNA content of the sample (total RNA content, for the Triton X-100-treated sample, or free (i.e., unencapsulated RNA for the non-treated sample) is based on the signal of the RiboGreen® dye, as measured using a spectrofluorophotometer. RNA encapsulation is calculated by comparing the RiboGreen® signals of the RNA-LNP samples in the absence (free RNA) and presence (total RNA) of Triton™ X-100.

| | | | Sub-visible particle | | | |
|---|---|---|---|---|---|---|
| | | | count | | | |
| | | Osmo | Number of | Number of | RNA | |
| Size | | (mOsmoL/ | particles ≥ | particles ≥ | conc., | EE |
| (PDI) | pH | kg) | 10 μm | 25 μm | (mg/mL) | (%) |
| 71 nm | 5.4 | 374 | 36 | 1 | 0.1 | 99.7 |
| (0.19) | | | | | | |

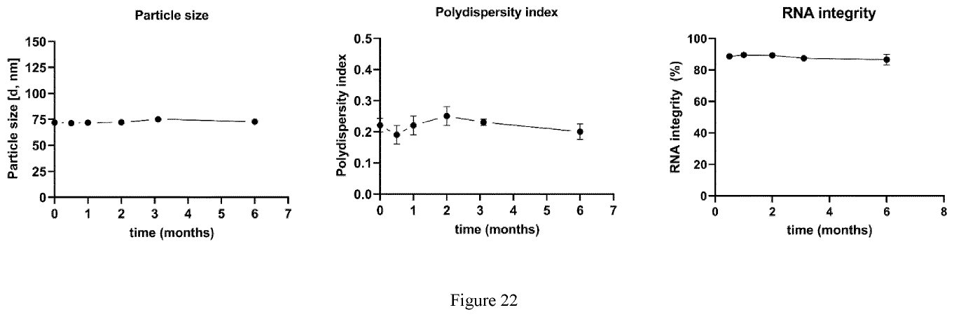

Encapsulation efficiency (EE) for the drug product is more than 99% (Table 3). As shown in Table 3 and FIG. 21, RNA-LNPs having good particle characteristics could be manufactured starting from pre-LNPs prepared out of ALC-0315 lipid in 5 mM acetic acid, 10% sucrose (w/v). The freeze thaw stability of the formulation at −20° C. was very good with no significant increase in particle size and PDI (FIG. 21). The critical quality attributes also during long term storage of RNA-LNPs at −20° C., were monitored over a period of 6 months. As shown in FIG. 22, the particle size, polydispersity and RNA integrity remained controlled for the entire duration of the study.

### Example 18: RNA-LNPs from Pre-LNPs of Example 12 in 1.25 mM Malic Acid, 10% Sucrose

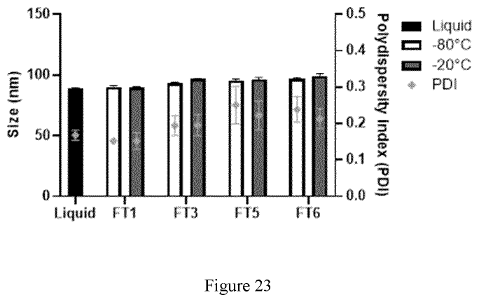

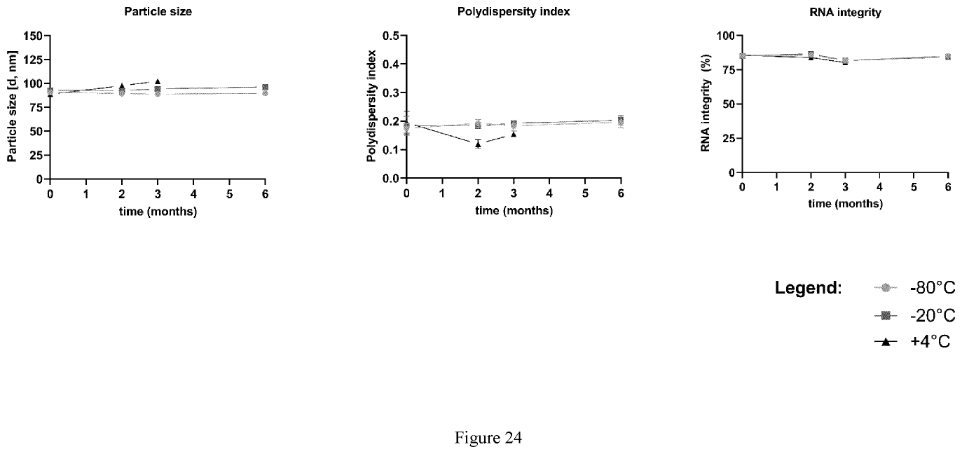

The pre-LNP phase was in 1.25 mM malic acid in 10% sucrose (w/v) at pH 4.5, with a final concentration of ALC-0315 of 4.6 mM. The raw RNA-LNPs had a lipid-to-RNA ratio of 6 and an RNA concentration of 0.125 mg/mL. The storage matrix was composed of 30 mM HEPES, 7 mM Tris, 30% sucrose, pH 7 to final drug product target pH 5.3 and target RNA concentration of 0.1 mg/mL. No significant increase in particle size or PDI was observed for up to six freeze thaw cycles for both −20° C. and −80° C. storage conditions, indicating a good frozen storage stability (see FIG. 23). Encapsulation efficiency (EE) for the RNA-LNPs manufactured from pre-LNP with 1.25 mM malic acid in 10% sucrose (w/v) was more than 99% (see Table 4). As shown in FIG. 23 and Table 4, RNA-LNPs having good particle characteristics could be manufactured starting from pre-LNPs comprising ALC-0315 in 1.25 mM Malic acid, 10% sucrose. Colloidal stability of the LNPs manufactured with the described process was very promising, the particle sizes and polydispersity remained controlled up to six freeze thaw cycles.

TABLE 4
Size, PDI, pH, Osmo, SVP, EE, for the RNA-LNPs.

| | | | Sub-visible particle | | | |
|---|---|---|---|---|---|---|
| | | | count | | | |
| | | Osmo | Number of | Number of | RNA | |
| Size | | (mOsmoL/ | particles ≥ | particles ≥ | conc., | EE |
| (PDI) | pH | kg) | 10 μm | 25 μm | (mg/mL) | (%) |
| 93 nm | 5.2 | 389 | 18 | 0 | 0.1 | 99.5 |
| (0.18) | | | | | | |

The long-term stability of RNA-LNPs manufactured from pre-LNPs from with ALC-0315 lipid and in 1.25 mM malic acid, 10% sucrose, stored at acidic pH regime at ~5.2, in frozen conditions both at −20° C. and −80° C., and liquid conditions +4° C. were followed. The critical quality attributes, such as particle size, polydispersity (PDI), and RNA integrity were monitored. The critical quality attributes remained controlled for a period 6 months at −20° C. and −80° C., and 3 months at 4° C.

### Example 19: RNA-LNPs from preLNPs of Example 11 in 5 mM Acetic Acid, 10% Sucrose

Pre-LNPs were composed of BHD-C2C2-PipZ:cholesterol:DSPC:α-tocopherol pAEEA14 lipids in a molar ratio 47.5:38.5:10:4 at a final concentration of BHD-C₂C₂-PipZ lipid of 5 mM. The pre-LNPs were provided in 5 mM acetic acid in 10% sucrose (w/v) at pH ~4.5. As shown in Table 5, RNA-LNPs having good particle characteristics could be manufactured starting from pre-LNPs prepared out of BHD-C2C2-PipZ lipid in 5 mM acetic acid, 10% sucrose.

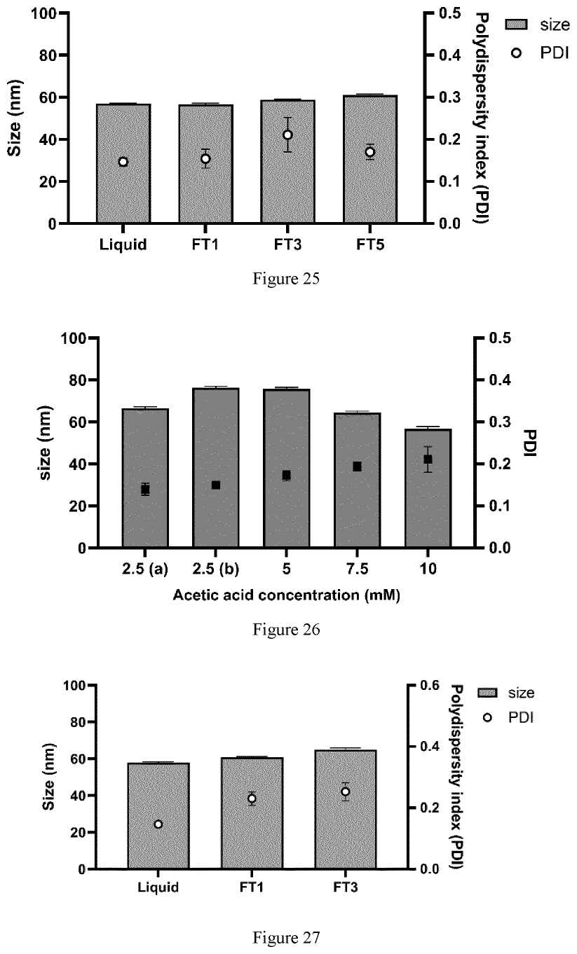

Encapsulation efficiency (EE) was measured following the protocol described above and the RNA encapsulation efficiency was 100% (Table 5). Additionally, the freeze-thaw stability of the formulation at −20° C. was very good as no increase of particle size and PDI were observed (FIG. 25).

TABLE 5
Size, PDI, pH, Osmo, SVP, EE for RNA-LNPs after
one freeze thaw cycle at −20° C.

| | | | Sub-visible particle | | | |
|---|---|---|---|---|---|---|
| | | | count | | | |
| | | Osmo | Number of | Number of | RNA | |
| Size | | (mOsmoL/ | particles ≥ | particles ≥ | conc., | EE |
| (PDI) | pH | kg) | 10 μm | 25 μm | (mg/mL) | (%) |
| 57 nm | 5.3 | 333 | 2 | 0 | 0.1 | 100 |
| (0.15) | | | | | | |

### Example 20: RNA-LNPs from preLNPs of Example 11 in 2.5 to 10 mM Acetic Acid in Sucrose

Pre-LNPs were composed of BHD-C2C2-PipZ:cholesterol:DSPC: α-tocopherol pAEEA14 lipids in a molar ratio 47.5:40.7:10:1.8 at a final concentration of BHD-C₂C₂-PipZ ionizable lipid of 5 mM. The pre-LNPs were provided in 2.5 mM, 5 mM, 7.5 mM, or 10 mM acetic acid in 10% (w/v) sucrose, pH 4.5. The raw RNA-LNPs were further processed by dilution with a storage matrix to dilute the drug product to RNA concentration of 0.10 mg/mL. For final drug product (DP) in (i) acidic conditions (pH<6) a storage matrix of 60 mM HEPES, 3 mM Tris, 30% sucrose (w/v), pH 6.3 was used; (ii) physiological conditions (pH >7) a storage matrix of 50 mM Tris, 30% sucrose, pH 8.5 was used.

TABLE 6
Details of pH values of manufactured RNA-LNPs.

| | | pH | | |
|---|---|---|---|---|
| Acetic acid | | Raw | DP diluted using 60 | DP diluted using |
| concentration | | RNA- | mM HEPES, 3 mM Tris, | 50 mM Tris, 30% |
| in preLNPs | | LNP | 30% sucrose at pH 6.3 | sucrose at pH 8.5 |
| 2.5 | mM | 5.5 | 5.8 | 7.8 |
| 5 | mM | 4.8 | 5.1 | n.a. |
| 7.5 | mM | 4.6 | 4.8 | n.a. |
| 10 | mM | 4.4 | 4.6 | n.a. |

As shown in FIG. 26, RNA-LNPs could be manufactured from preLNPs of varying acetic acid concentrations (i.e. 2.5 to 10 mM). A general trend of lower RNA-LNP particle size with increasing amount of acidifier was observed, for example RNA-LNPs of 57 nm or 65 nm could be obtained using pre-LNPs of acetic concentration of either 10 mM or 2.5 mM acetic acid, respectively. The RNA-LNPs diluted with 60 mM HEPES, 3 mM Tris, 30% sucrose had pH values below 6. The RNA-LNPs could also be prepared in a physiological pH regime using 50 mM Tris, 30% sucrose pH 8.5, as demonstrated for 2.5 mM acetic acid (Groups (a) and (b)). Furthermore, encapsulation efficiency was measured following the protocol described above and the RNA-LNP encapsulation efficiency amounted to 100% for all variants tested.

### Example 21: RNA-LNPs from preLNPs of Example 10 in 1.25 mM Acetic Acid in Sucrose

Pre-LNPs were composed of ALC-0315:cholesterol:DSPC:ALC-0159 lipids in a molar ratio 47.5:40.7:10:1.8 at a final concentration of ALC-0315 of 5 mM. The RNA phase was diluted in the storage buffer composed of 10 mM HEPES, 0.1 mM EDTA, pH 6 (a lower pH was used to compensate for the reduced concentration of AcOH in the pre-LNP phase), at the target concentration of 0.25 mg/mL. The pre-LNPs were provided in 1.25 mM acetic acid, 10% sucrose (w/v), pH 4.5.

TABLE 7
Size, PDI, pH, Osmo, SVP, EE for the RNA-LNPs after
one freeze-thaw cycle at −20° C.

| | | | Sub-visible particle | | | |
|---|---|---|---|---|---|---|
| | | | count | | | |
| | | Osmo | Number of | Number of | RNA | |
| Size | | (mOsmoL/ | particles ≥ | particles ≥ | conc., | EE |
| (PDI) | pH | kg) | 10 μm | 25 μm | (mg/mL) | (%) |
| 61 nm | 5.3 | 351 | 3 | 0 | 0.1 | 80 |
| (0.23) | | | | | | |

EE of RNA-LNPs was evaluated using the RiboGreen® assay as described in Example 17 and was acceptable at around 80% (Table 7). As shown in FIG. 27 and Table 7, RNA-LNPs having good particle characteristics could be manufactured starting from pre-LNPs in the lower amount of 1.25 mM acetic acid. The particle size and polydispersity remained controlled under freeze thaw stress (FIG. 27).

### Example 22: RNA-LNPs from Pre-LNPs of Example 10 in 2.5 mM Acetic Acid in Sucrose

The pre-LNP phase was provided in 2.5 mM acetic acid, 10% sucrose (w/v), pH 4.5 at a final concentration of ALC-0315 of 5 mM. The raw RNA-LNPs were further processed by dilution with a storage matrix composed of 60 mM HEPES, 30% sucrose (w/v), pH 4.5 to target pH of the final drug product of 5.5.

TABLE 8
Size, PDI, pH, Osmo, SVP, EE for the RNA-LNPs after
one freeze thaw cycle at −20° C.

| | | | Sub-visible particle | | | |
|---|---|---|---|---|---|---|
| | | | count | | | |
| | | Osmo | Number of | Number of | RNA | |
| Size | | (mOsmoL/ | particles ≥ | particles ≥ | conc., | EE |
| (PDI) | pH | kg) | 10 μm | 25 μm | (mg/mL) | (%) |
| 78 nm | 5.5 | 356 | 89 | 18 | 0.09 | 100 |
| (0.14) | | | | | | |

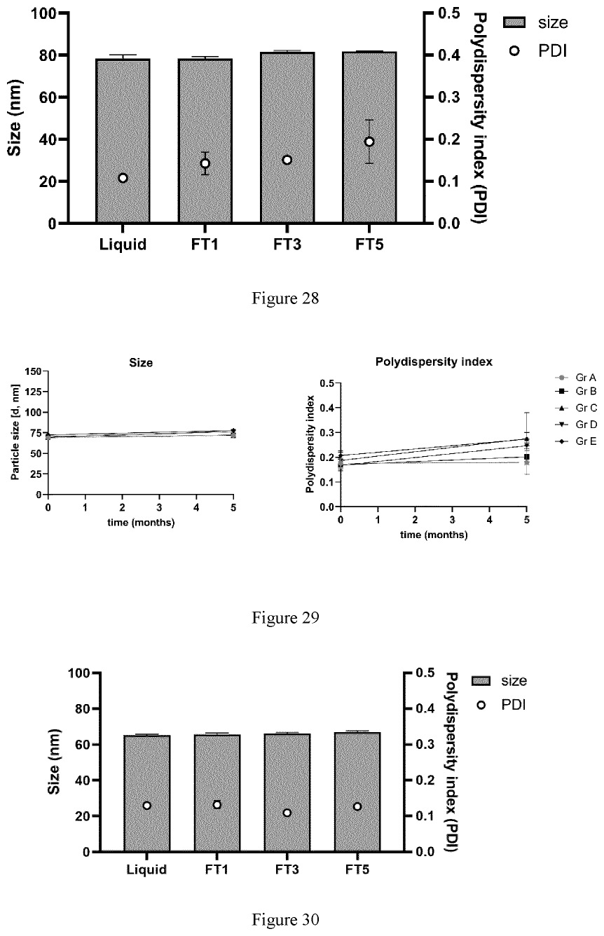

As shown in FIG. 28 and Table 8, RNA-LNPs having good particle characteristics could be manufactured also in 2.5 mM acetic acid, 10% sucrose. EE of the RNA-LNPs was evaluated using the RiboGreen® assay described in Example 17, and was measured to be 100% (Table 8). The freeze thaw stability of the formulation at −20° C. was very good as no increase of particle size and PDI were observed (FIG. 28).

### Example 23: RNA-LNPs from preLNPs of Example 10 in 5 mM Acetic Acid in Sucrose, with Up-Concentration

The pre-LNP phase was provided in 5 mM acetic acid, 10% sucrose (w/v), pH ~4.5, at a final concentration of ALC-0315 of 5 mM. The raw RNA-LNPs obtained directly after mixing were up-concentrated to an RNA concentration of 0.20 mg/mL, 0.30 mg/mL, 0.76 mg/mL, or 1.61 mg/mL. RNA concentration measurements were performed by applying ultraviolet-visible spectroscopy technique on the sample following treatment with zwittergent-ethanol solution to disrupt particles and induce RNA release. The up-concentrated raw RNA-LNPs were further processed by dilution with a storage matrix composed of 60 mM HEPES, 3 mM Tris, 30% sucrose at pH 6.3 to target pH of the final drug product of 5.5 and the final sucrose concentration of 10% (w/v).

TABLE 9
Size, PDI, pH, Osmo, for RNA-LNPs after one
freeze thaw cycle at −20° C.

| | RNA conc. | | | Osmo | RNA conc. |
|---|---|---|---|---|---|
| | raw LNP | | | (mOsmoL/ | final LNP |
| Group | (mg/mL) | Size (PDI) | pH | kg) | (mg/mL) |
| Gr A | 0.1 | 68 nm (0.12) | 5.3 | 370 | 0.1 |
| Gr B | 0.20 | 69 nm (0.12) | 5.3 | 372 | 0.2 |
| Gr C | 0.30 | 68 nm (0.14) | 5.3 | 381 | 0.3 |
| Gr D | 0.8 | 69 nm (0.16) | 5.1 | 386 | 0.6 |
| Gr E | 1.7 | 69 nm (0.18) | 5.1 | 394 | 1.3 |

As shown in FIG. 29 and Table 9, up-concentrated RNA-LNPs showed good particle characteristics. Furthermore, the long-term stability of these up-concentrated RNA-LNPs stored at −20° C., pH 5.4 was assessed. The critical quality attributes, such as particle size and PDI, were monitored and remained controlled for a period of 5 months at −20° C. (see FIG. 29).

### Example 24: RNA-LNPs from preLNPs of Example 10 in 5 mM Acetic Acid Using Trehalose or Dextrose as Cryoprotectant

The pre-LNP phase was provided in (A) 5 mM acetic acid, 10% trehalose (w/v), pH 4.5, or (B) 5 mM acetic acid, 5% dextrose (w/v), pH 4.5 at a final concentration of ALC-0315 of 5 mM. The raw RNA-LNPs were further processed by dilution with a storage matrices composed of: for pre-LNPs (A) 60 mM HEPES, 3 mM Tris, 30% trehalose (w/v), pH 6.3; or for pre-LNPs (B) 60 mM HEPES, 3 mM Tris, 15% dextrose (w/v), pH 6.3 to target pH of the final drug product around ~5.5 and target RNA concentration of 0.1 mg/mL.

As shown in Tables 10 and 11, RNA-LNPs having good particle characteristics could be manufactured starting from pre-LNPs composed of ALC-0315 lipid in 5 mM acetic acid in either 10% trehalose (w/v) or 5% dextrose (w/v). No particular effect of the cryoprotectant type on the RNA-LNP particles characteristics was observed and different cryoprotectant could be used interchangeably.

TABLE 10
Size, PDI, pH, Osmo, SVP for the RNA−LNPs from pre−LNPs
(A) after one freeze thaw cycle at −20° C.

| | | | Sub-visible particle count | | |
|---|---|---|---|---|---|
| | | Osmo | Number of | Number of | |
| | | (mOsmoL/ | particles ≥ | particles ≥ | RNA conc., |
| Size (PDI) | pH | kg) | 10 μm | 25 μm | (mg/mL) |
| 68 nm (0.15) | 5.1 | 309 | 62 | 0 | 0.1 |

TABLE 11
Size, PDI, pH, Osmo, SVP, for the RNA-LNPs from pre-LNPs
(B) after one freeze thaw cycle at −20° C.

| | | | Sub-visible particle count | | |
|---|---|---|---|---|---|
| | | Osmo | Number of | Number of | |
| | | (mOsmoL/ | particles ≥ | particles ≥ | RNA conc., |
| Size (PDI) | pH | kg) | 10 μm | 25 μm | (mg/mL) |
| 69 nm (0.19) | 5.2 | 316 | 18 | 0 | 0.1 |

### Example 25: RNA-LNPs from Lyophilized preLNPs of Example 13 in 5 mM AcOH in Sucrose

Pre-LNPs were manufactured as described in Example 13 and then lyophilized. The lyophilized pre-LNP cake was reconstituted in the appropriate amount of pure water, as estimated based on the weight loss upon pre-LNP drying. The pre-LNPs were allowed 30 min of equilibration time and then were filtered through 0.22 μm PES filter to be ready to use for RNA-LNP manufacturing, following the process outlined in Example 16. As shown in Table 12, RNA-LNPs having good particle characteristics could be manufactured from lyophilized and reconstituted pre-LNPs. EE of RNA-LNPs was evaluated using the RiboGreen® assay as described in Example 17 and measured to be 100%.

TABLE 12
Size, PDI, pH, Osmo, SVP, EE for the RNA-LNPs after
one freeze-thaw cycle at −20° C.

| | | | Sub-visible particle count | | |
|---|---|---|---|---|---|
| | | Osmo | Number of | Number of | |
| | | (mOsmoL/ | particles ≥ | particles ≥ | |
| Size (PDI) | pH | kg) | 10 μm | 25 μm | EE(%) |
| 65 nm (0.13) | 5.2 | 377 | 34 | 23 | 100 |

The freeze thaw stability of the formulation at −20° C. was very good as no increase of particle size and PDI were observed upon five freeze thaw cycles (FIG. 30). These data demonstrate successful use of lyophilized pre-LNPs for RNA-LNP manufacturing and suggests excellent long term frozen stability of the obtained RNA-LNP formulation stored in acidic conditions.

### Example 26: RNA-LNPs from preLNPs of Example 14 in 5 mM Acetic Acid and Different Concentrations of Sucrose

The pre-LNPs were provided in (A) 5 mM acetic acid, 5% sucrose (w/v), pH ~4.5; or (B) 5 mM acetic acid, 10% sucrose (w/v), pH ~4.5; or (C) 4 mM acetic acid in 20% sucrose (w/v) (obtained by dilution of pre-LNPs of (B) with a stock solution of 60% sucrose (w/v)).

The raw RNA-LNPs obtained directly after mixing had a lipid-to-RNA ratio of 6.6 for pre-LNP groups (A) and (B), or 5.3 for group (C). An RNA concentration of 0.125 mg/mL was obtained in all three cases. The raw RNA-LNPs were further processed by dilution with various storage matrices composed of 60 mM HEPES, 3 mM Tris, 40% sucrose (w/v), pH 6.3 (Group (A)); 60 mM HEPES, 3 mM Tris, 30% sucrose (w/v) at pH 6.3 (group (B)); 60 mM HEPES, 3 mM Tris, 10% sucrose (w/v), pH 6.3 (group (C)) to target pH of the final drug product of 5.5 and target the RNA concentration of 0.1 mg/mL in final formulation. A final drug product at physiological pH can also be obtained diluting the raw LNPs with a storage matrix of a higher pH. The final pH of the drug products tested are summarized in Table 13.

TABLE 13
Manufacturing conditions and storage matrices
used for preparing the RNA-LNPs.

| | | | Final |
|---|---|---|---|
| Group | preLNP phase | Storage matrix | pH |
| A | 5 mM acetic acid, 5% | 60 mM HEPES, 3 mM Tris, | 5.1 |
| | sucrose (w/v) | 40% sucrose, pH 6.3 | |
| B | 5 mM acetic acid, 10% | 60 mM HEPES, 3 mM Tris, | 5.2 |
| | sucrose (w/v) | 30% sucrose, pH 6.3 | |
| C | 4 mM acetic acid, 20% | 60 mM HEPES, 3 mM Tris, | 5.4 |
| | sucrose (w/v) | 10% sucrose, pH 6.3 | |

TABLE 14
Size, PDI, pH, Osmo, SVP, EE, for the RNA-LNPs.

| | | | | Sub-visible particle | | |
|---|---|---|---|---|---|---|
| | | | | count | | |
| | | | | Number of | Number of | |
| | | | Osmo | particles ≥ | particles ≥ | |
| Group | Size (PDI) | pH | (mOsmoL/kg) | 10 μm | 25 μm | EE (%) |
| A | 80 nm (0.18) | 5.1 | 384 | 23 | 2 | 100 |
| B | 72 nm (0.19) | 5.2 | 396 | 57 | 40 | 100 |
| C | 79 nm (0.19) | 5.4 | 389 | 34 | 2 | 100 |

RNA-LNPs having good particle characteristics could be manufactured starting from pre-LNPs comprising different sucrose concentrations. EE of RNA-LNPs was evaluated using the RiboGreen® assay as described in Example 17 and measured to be 100% (Table 14).

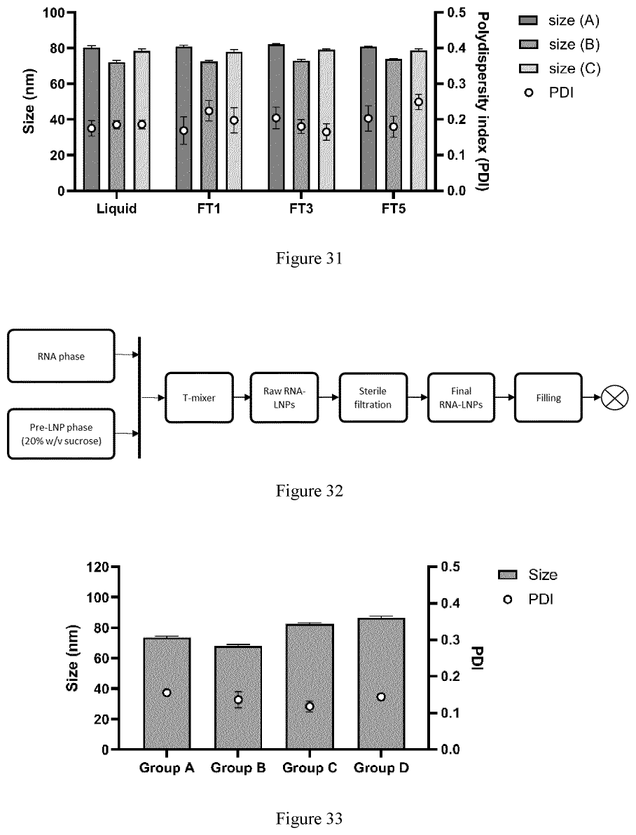

The freeze-thaw stability of the formulations of Groups (A), (B) and (C) at −20° C. was very good as no increase of particle size and PDI were observed upon five freeze-thaw cycles (FIG. 31). These data can serve as an indication of very good long term frozen stability of the formulations at −20° C. Similar frozen stability is also expected for RNA-LNPs stored at physiological pH regime (pH ≥7.0).

### Example 27: Further Simplified RNA-LNP Manufacturing from Pre-LNPs Containing 20% Sucrose

A further simplified manufacturing process for formation of RNA-LNPs was developed by providing a pre-LNP phase comprising 20% sucrose, for example following an exemplary manufacturing scheme as shown in FIG. 32. The pre-LNP phase was provided in 20% sucrose (w/v), so that 1:1 mixing of the different RNA phases (Groups A to D) and the pre-LNP phase results in RNA-LNPs containing 10% sucrose in the respective storage matrix.

Surprisingly, RNA could be effectively loaded into a pre-LNP phase comprising the higher concentration of 20% sucrose, despite the increased viscosity of the pre-LNP phase, to produce RNA-LNPs (FIG. 33).

The particle size and PDI of the manufactured RNA-LNPs was measured. By adjusting the pH of the RNA and pre-LNP phases, the pH of the resulting final RNA-LNP formulations can be either below pH 6 (acid regime) or above pH 7 (physiological regime). Depending on the type and concentration of the excipients in the RNA phase, the LNP particle size can be controlled, for example for groups A to D particle size was found range from 60 to 100 nm (FIG. 33). Particle attributes may be further modulated by optimizing the conditions used for the pre-LNP and RNA phases. However, these data demonstrate the feasibility of the exemplary simplified manufacturing scheme, which allow for omitting a further dilution step for the RNA-LNPs. Shortening the manufacturing process for the RNA-LNP phase advantageously minimizes the impact on RNA integrity and other product attributes.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in chemistry, biochemistry, molecular biology, biotechnology or related fields are intended to be within the scope of the following claims.

1. An aqueous dispersion having an aqueous mobile phase and a dispersed phase; wherein:
the dispersed phase comprises a lipid mixture including a cationically ionisable lipid; and
the aqueous mobile phase comprises an anion of an aqueous acid;
wherein the aqueous dispersion is substantially free of inorganic cations, organic solvents and RNA, and
wherein the aqueous mobile phase comprises a cryoprotectant.

2. The aqueous dispersion of claim 1, having a pH of 2.5 to 5.5.

3. The aqueous dispersion of claim 2, having a pH of 2.5 to 4.5.

4. The aqueous dispersion of any one of claims 1 to 3, wherein the molar ratio between the cationic or cationically ionizable lipid and anions of aqueous acid is between 20:1 and 1:20.

5. The aqueous dispersion of any one of claims 1 to 4, wherein the molar ratio between the cationic or cationically ionizable lipid and anions of aqueous acid is between 5:1 and 1:5.

6. The aqueous dispersion of any one of claims 1 to 5, wherein the aqueous acid is an inorganic acid or a water-soluble organic acid.

7. The aqueous dispersion of any one of claims 1 to 6, wherein the aqueous acid is acetic acid, malic acid, or succinic acid.

8. The aqueous dispersion of any one of claims 1 to 7, wherein the concentration of the aqueous acid is in the range of 1-20 mM.

9. The aqueous dispersion of any one of claims 1 to 8, wherein the concentration of the aqueous acid is in the range of 2.5 to 10 mM.

10. The aqueous dispersion of any one of claims 1 to 9, wherein the cationic or cationically ionisable lipid is selected from the group consisting of:
[(4-hydroxybutyl)azanediyl]di(hexane-6,1-diyl) bis(2-hexyldecanoate) (ALC-315);
1,2-dioleoyloxy-3-dimethylaminopropane (DODMA);
2,2-dilinoleyl-4-dimethylaminoethyl-[1,3]-dioxolane (DLin-KC2-DMA);
heptatriaconta-6,9,28,31-tetraen-19-yl-4-(dimethylamino)butanoate (D-Lin-MC3-DMA);
1,2-dilinoleyloxy-N,N-dimethylaminopropane (DLin-DMA);
di((Z)-non-2-en-1-yl)-9-((4-(dimethylaminobutanoyl)oxy)heptadecanedioate (L319);
bis-(2-butyloctyl) 10-(N-(3-(dimethylamino)propyl)nonanamido)-nonadecanedioate (A9);
(heptadecan-9-yl 8-{(2-hydroxyethyl)[6-oxo-6-(undecyloxy)octyl]amino}-octanoate) (L5);
heptadecan-9-yl 8-{(2-hydroxyethyl)[6-oxo-6-(undecyloxy)hexyl]amino}-octanoate) (SM-102);
O—[N-{(9Z,12Z)-octadeca-9,12-dien-1-yl)}-N-{7-pentadecylcarbonyloxyoctyl}-amino]4-(dimethylamino)butanoate (HY501);
2-(di-((9Z,12Z)-octadeca-9,12-dien-1-yl)amino)ethyl 4-(dimethylamino)butanoate (EA-2);
4-((di-((9Z,12Z)-octadeca-9,12-dien-1-yl)amino)oxy)-N,N-dimethyl-4-oxobutan-4-amine (HYAM-2);
((2-(4-(dimethylamino)butanoyl)oxy)ethyl)azanediylbis(octane 8,1-diyl) bis(2-hexyldecanoate) (EA-405);
(2-(4-(dimethylamino)butanoyl)oxy)azanediylbis(octane 8,1-diyl) bis(2-hexyldecanoate) (HY-405);
di(heptadecan-9-yl) 3,3′-((2-(4-methylpiperazin-1-yl)ethyl)azanediyl)dipropionate (BHD-C2C2-PipZ);
bis(2-octyldodecyl) 3,3′-((2-(1-methylpyrrolidin-2-yl)ethyl)azanediyl)dipropionate (BODD-C2C2-1Me-Pyr);
7,7′-((4-hydroxybutyl)azanediyl)-bis(N-hexyl-N-octylheptane-1-sulfonamide) (BNT-51);
7,7′-((4-(3,3-dimethylthioureido)butyl)azanediyl)bis(N-hexyl-N-octylheptane-1-sulfonamide) (BNT-52);
the compound having the structure 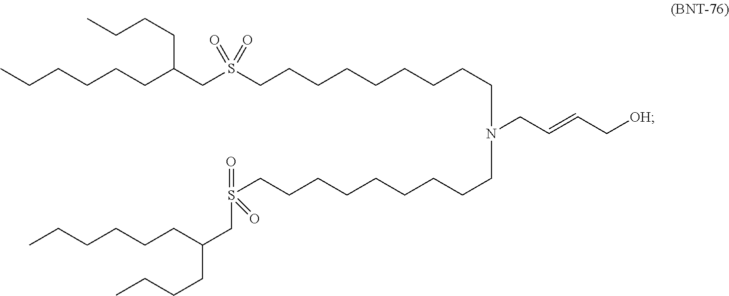 [SMILES: CCCCCCC(CCCC)CS(=O)(=O)CCCCCCCCCN(C/C=C/CO)CCCCCCCCCS(=O)(=O)CC(CCCC)CCCCCC]
or a mixture of any thereof.

11. The aqueous dispersion of any one of claims 1 to 10, wherein the lipid mixture further comprises one or more additional lipids.

12. The aqueous dispersion of claim 11, wherein the one or more additional lipids comprise a neutral or zwitterionic lipid.

13. The aqueous dispersion of claim 12, wherein the one or more additional lipids comprise a neutral or zwitterionic phospholipid.

14. The aqueous dispersion of claim 13, wherein the neutral or zwitterionic phospholipidis selected from the group consisting of:
distearoylphosphatidylcholine (DSPC);
dioleoylphosphatidylcholine (DOPC);
dimyristoylphosphatidylcholine (DMPC);
dipalmitoylphosphatidylcholine (DPPC);
palmitoyloleoyl-phosphatidylcholine (POPC);
dioleoylphosphatidylethanolamine (DOPE);
1,2-di-(9Z-octadecenoyl)-sn-glycero-3-phosphocholine (DOPG);
N-palmitoyl-D-erythro-sphingosylphosphorylcholine (SM);
or a mixture of any thereof.

15. The aqueous dispersion of claim 14, wherein the neutral or zwitterionic phospholipid is distearoylphosphatidylcholine (DSPC).

16. The aqueous dispersion of any one of claims 11 to 15, wherein the one or more additional lipids comprise a steroid.

17. The aqueous dispersion of claim 16, wherein the steroid is cholesterol.

18. The aqueous dispersion of any one of claims 11 to 15, wherein the one or more additional lipids comprise a grafted lipid.

19. The aqueous dispersion of claim 18, wherein the grafted lipid is selected from the group consisting of a poly(alkylene glycol)-conjugated lipid, a poly(sarcosinate)-conjugated lipid, a poly(oxazoline) (POX)-conjugated lipid; a poly(oxazine) (POZ)-conjugated lipid; a poly(vinyl pyrrolidone) (PVP)-conjugated lipid; a poly(N-(2-hydroxypropyl)-methacrylamide) (pHPMA)-conjugated lipid; a poly(dehydroalanine) (pDha)-conjugated lipid; a poly(aminoethoxy ethoxy acetic acid) (pAEEA)-conjugated lipid; and a poly(2-methylaminoethoxy ethoxy acetic acid) (pmAEEA)-conjugated lipid; or a mixture of any thereof.

20. The aqueous dispersion of any one of claims 11 to 19, wherein the one or more additional lipids comprise a peptide-conjugated lipid.

21. The aqueous dispersion of claim 20, wherein the peptide-conjugated lipid is an ALFA-tag conjugated lipid.

22. A lyophilised composition comprising the aqueous dispersion of any one of claims 1 to 21.

23. A frozen composition comprising the aqueous dispersion of any one of claims 1 to 21, wherein the frozen composition is at a temperature between −15° C. to −90° C.

24. A method of forming the aqueous dispersion of any one of claims 1 to 21, the method comprising mixing:
(i) a lipid mixture comprising a cationically ionisable lipid;
(ii) an aqueous phase comprising an aqueous acid and a cryoprotectant;
to produce the aqueous dispersion comprising an anion of the aqueous acid.

25. A method of forming the aqueous dispersion of any one of claims 1 to 23, the method comprising:
(a) mixing:
(i) a lipid mixture comprising a cationically ionisable lipid; and
(ii) an aqueous phase comprising an aqueous acid;
to produce a first intermediate aqueous dispersion comprising an anion of the aqueous acid; and
(b) adding the cryoprotectant to the first intermediate aqueous dispersion to produce the aqueous dispersion.

26. A method of forming a lipid particle containing RNA, the method comprising mixing the aqueous dispersion of any one of claims 1 to 21 with an aqueous solution comprising RNA, to produce the lipid particle containing RNA.

27. The method of claim 26, wherein the cryoprotectant in the aqueous dispersion is selected from the group consisting of sucrose, trehalose or glucose, or a mixture thereof.

28. The method of claim 27, wherein the cryoprotectant is sucrose or trehalose and is present in the aqueous dispersion at a concentration of about 15% to about 25%.

29. The method of any one of claims 26 to 28, wherein the method comprises no further dialysis, filtration, dilution or addition of cryoprotectant steps.

30. The method of any one of claims 26 to 29, wherein the RNA is mRNA.

31. The method of claim 30, wherein the mRNA encodes one or more patient-specific antigens suitable for personalized cancer therapy.

32. A lipid particle comprising RNA obtained or obtainable by the method of any one of claims 26 to 31.

33. A lipid particle according to claim 32, which is a lipid nanoparticle.

34. A lipid particle of claim 32 or 33 for use in medicine.

35. A lipid particle of claim 32 or 33 for use in treating cancer.


SEQUENCE LISTING (17 sequences)

SEQ ID NO: 1 - protein, 15 aa, synthetic construct
  VARIANT 1: note = X is P or deleted
  VARIANT 2: note = X is S, G, T, or P
  VARIANT 3: note = X is R, G, A, E, or P
  VARIANT 8: note = X is L, I, or V
  VARIANT 9: note = X is R, A, Q, or E
  VARIANT 10: note = X is R, A, Q, or E
  VARIANT 13: note = X is T, S, N, E, P, A, or deleted
  VARIANT 14: note = X is E, K, P, S, A, N, or deleted
  VARIANT 15: note = X is P or deleted
  VARIANT 4: note = X is L, I, or V
XXXXZZZXXXRLXXX

SEQ ID NO: 2 - protein, 13 aa, synthetic construct
SRLEEELRRRLTE

SEQ ID NO: 3 - protein, 14 aa, synthetic construct
PSRLEEELRRRLTE

SEQ ID NO: 4 - protein, 14 aa, synthetic construct
SRLEEELRRRLTEP

SEQ ID NO: 5 - protein, 15 aa, synthetic construct
PSRLEEELRRRLTEP

SEQ ID NO: 6 - protein, 16 aa, synthetic construct
  VARIANT 1: note = X is P or deleted
  VARIANT 2: note = X is S, G, T, or P
  VARIANT 4: note = X is L, I, or V
  VARIANT 3: note = X is R, G, A, E, or P
  VARIANT 8: note = X is L, I, or V
  VARIANT 9: note = X is R, A, Q, or E
  VARIANT 10: note = X is R, A, Q, or V
  VARIANT 13: note = T, S, N, E, P, A, or deleted
  VARIANT 14: note = X is E, K, P, S, A, N, or deleted
  VARIANT 15: note = X is P or deleted
  CROSSLNK 1..15: note = The side chains of any two of the amino acids 1 to 15 ,X1 and X2, are connected covalently. The amino acids which are not X1 and X2 are as defined.
XXXXZZZXXXXRLXXX

SEQ ID NO: 7 - protein, 13 aa, synthetic construct
  CROSSLNK 5..9: note = Amino acids 5 and 9 are connected covalently
SRLEEELRKRLTE

SEQ ID NO: 8 - protein, 13 aa, synthetic construct
  CROSSLNK 5..9: note = Amino acids 5 and 9 are connected covalently
SRLEDELRKRLTE

SEQ ID NO: 9 - protein, 13 aa, synthetic construct
  CROSSLNK 5..8: note = Amino acids 5 and 8 are connected covalently.
SRLEEELKRRLTE

SEQ ID NO: 10 - protein, 13 aa, synthetic construct
  CROSSLNK 9..13: note = Amino acids 9 and 13 are covalently connected.
SRLEEELRKRLTE

SEQ ID NO: 11 - protein, 13 aa, synthetic construct
  VARIANT 5: note = X5 is covalently connected to X9
  VARIANT 9: note = X9 is covalently connected to X5
SRLEXELRXRLTE

SEQ ID NO: 12 - protein, 12 aa, synthetic construct
  VARIANT 3: note = X is I or V
VTXSALNAMAMG

SEQ ID NO: 13 - protein, 9 aa, synthetic construct
  VARIANT 4: note = X is E, H, N, D, or S
AVSXRGNAM

SEQ ID NO: 14 - protein, 11 aa, synthetic construct
LEDRVDSFHDY

SEQ ID NO: 15 - protein, 13 aa, synthetic construct
  VARIANT 4: note = X is I or V
GVTXSALNAMAMG

SEQ ID NO: 16 - protein, 9 aa, synthetic construct
  VARIANT 4: note = X is E, H, N, D, or S
AVSXRGNAM

SEQ ID NO: 17 - protein, 122 aa, synthetic construct
EVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSS